(19) United States
(12) Patent Application Publication
(10) Pub. No.: US 2021/0332377 A1
(43) Pub. Date: Oct. 28, 2021

(54) GENETICALLY ENGINEERED PLANTS THAT EXPRESS A QUINONE-UTILIZING MALATE DEHYDROGENASE

(71) Applicant: YIELD10 BIOSCIENCE, INC., Woburn, MA (US)

(72) Inventors: Frank Anthony SKRALY, Watertown, MA (US); Jihong TANG, West Roxbury, MA (US); Kristi D. SNELL, Belmont, MA (US); Meghna MALIK, Saskatoon (CA)

(21) Appl. No.: 17/284,135

(22) PCT Filed: Oct. 10, 2019
(86) PCT No.: PCT/US2019/055575

§ 371 Date: Apr. 9, 2021

Related U.S. Application Data
(60) Provisional application No. 62/745,134, filed on Oct. 12, 2018.

(51) Int. Cl. C12N 15/82; C12N 9/04

(52) U.S. Cl. CPC C12N 15/8261; C12N 9/0006; C07K 2319/07; C12Y 101/05004; C12N 15/8213

(57) ABSTRACT

Genetically engineered plants that express a quinone-utilizing malate dehydrogenase (MQO) are provided. The plant comprises a modified gene for the quinone-utilizing malate dehydrogenase. The modified gene comprises (i) a promoter and (ii) a nucleic acid sequence encoding the quinone-utilizing malate dehydrogenase. The promoter is non-cognate with respect to the nucleic acid sequence encoding the quinone-utilizing malate dehydrogenase. The modified gene is configured such that transcription of the nucleic acid sequence is initiated from the promoter and results in expression of the quinone-utilizing malate dehydrogenase. The plants can express the quinone-utilizing malate dehydrogenase in mitochondria of cells of the plants. Conversion of malate to oxaloacetate in the mitochondria can be increased, resulting in increased crop performance and/or seed, fruit or tuber yield. Methods and compositions for making the plants also are provided.

### FIELD OF THE INVENTION

The present invention relates generally to genetically engineered plants that express a quinone-utilizing malate dehydrogenase (also termed “MQO protein,” “MQO enzyme,” or “malate:quinone oxidoreductase”), and more particularly to such genetically engineered plants with increased expression of the quinone-utilizing malate dehydrogenase in mitochondria of cells of the plants, resulting in increased crop performance and/or seed, fruit, or tuber yield.

### BACKGROUND OF THE INVENTION

The world faces a major challenge in the next 35 years to meet the increased demands for food production to feed a growing global population, which is expected to reach 9 billion by the year 2050. Food output will need to be increased by up to 70% in view of the growing population, increased demand for improved diet, land use changes for new infrastructure, alternative uses for crops and changing weather patterns due to climate change. Studies have shown that traditional crop breeding alone will not be able to solve this problem (Deepak K. Ray, Nathaniel D. Mueller, Paul C. West and Jonathon A. Foley, 2013. Yield trends are Insufficient to Double Global Crop Production by 2050. PLOS, published Jun. 19, 2013 doi.org/10.1371/journal.pone.0066428). There is therefore a need to develop new technologies to enable step change improvements in crop performance and in particular crop productivity and/or yield.

Major agricultural crops include food crops, such as maize, wheat, oats, barley, soybean, millet, sorghum, pulses, bean, tomato, corn, rice, cassava, sugar beets, and potatoes, forage crop plants, such as hay, alfalfa, and silage corn, and oilseed crops, such as camelina, Brassica species (e.g. B. napus (canola), B. rapa, B. juncea, and B. carinata), crambe, soybean, sunflower, safflower, oil palm, flax, and cotton, among others. Productivity of these crops, and others, is limited by numerous factors, including for example relative inefficiency of photochemical conversion of light energy to fixed carbon during photosynthesis, as well as loss of fixed carbon by photorespiration and/or other essential metabolic pathways having enzymes catalyzing decarboxylation reactions. For seed (grain), tuber or fruit crops, the ratio of seed, tubers or fruit produced per unit plant biomass (also referred to as the harvest index) is also a major determinant of crop productivity.

Increasing seed, fruit or tuber yield in major crops can be viewed as a two-step carbon optimization problem, the first is improving photosynthetic carbon fixation and the second is optimizing the flow of fixed carbon to seed production versus vegetative biomass (roots, stems, leaves etc.). The ratio of harvested seed to the total above ground biomass is also described as the harvest index. Increasing the harvest index of seed, fruit and tuber crops is also an objective of this invention.

During seed production in plants, the tricarboxylic acid (TCA) cycle is expected to operate in the mitochondria to provide NADH and ATP from sugar metabolism. In that case, malate must be converted to oxaloacetate by malate dehydrogenase (MDH). Plants typically contain only NAD(P)H-dependent MDHs, and these enzymes catalyze reactions with thermodynamics that greatly favor malate formation, even when the [NAD⁺]/[NADH] ratio is high. MDHs with NAD(P)H as cofactor are soluble enzymes and thus are not linked directly to respiration as is succinate dehydrogenase, which can proceed in an unfavorable thermodynamic direction with ease because it is coupled to a reaction (donation of electrons to oxygen) that is extremely favorable, such that the overall thermodynamics actually favor electron donation.

It is therefore an objective of this invention to provide genes, systems and plants having a thermodynamically more favorable system for converting malate to oxaloacetate (OAA) by increasing expression of a protein having the activity of a quinone-utilizing malate dehydrogenase (Mqo; EC 1.1.5.4). In a preferred embodiment the expressed MQO protein is operably linked to a peptide signal such that it is targeted to the mitochondrion of the plant cells. It is expected that plants which have been engineered to have the higher levels of Mqo expression in the mitochondria have better performance and/or higher seed yield than the same plant which has not been engineered to increase Mqo expression.

### BRIEF SUMMARY OF THE INVENTION

Methods, genes and systems for producing plant cells, tissues and plants having increased expression of a quinone-utilizing malate dehydrogenase (Mqo; EC 1.1.5.4) are disclosed. The plant cells, tissues and plants comprise increased expression of a quinone-utilizing malate dehydrogenase (Mqo; EC 1.1.5.4) in the mitochondria such that the conversion of malate to oxaloacetate is increased, resulting in increased crop performance and/or yield. The genes encoding the quinone-utilizing malate dehydrogenase (Mqo; EC 1.1.5.4) can be used alone or in combination with altered expression of additional genes to enhance photosynthesis or carbon partitioning to seed. The expression of the genes encoding the quinone-utilizing malate dehydrogenase (Mqo; EC 1.1.5.4) proteins can be increased using genetic engineering techniques or marker assisted breeding approaches to develop plants with increased performance and/or yield. Where genetic engineering techniques are used to increase the expression of the quinone-utilizing malate dehydrogenase (Mqo; EC 1.1.5.4) proteins, the increased expression can be accomplished using transgenic technologies with quinone-utilizing malate dehydrogenase (Mqo; EC 1.1.5.4) genes from a source other than the plant being modified, or by genome editing approaches to increase the expression of the plant MQO genes in constitutive, seed-specific, and/or seed-preferred manners.

Thus, a genetically engineered plant that expresses a quinone-utilizing malate dehydrogenase is disclosed. The genetically engineered plant comprises a modified gene for the quinone-utilizing malate dehydrogenase. The modified gene comprises (i) a promoter and (ii) a nucleic acid sequence encoding the quinone-utilizing malate dehydrogenase. The promoter is non-cognate with respect to the nucleic acid sequence encoding the quinone-utilizing malate dehydrogenase. The modified gene is configured such that transcription of the nucleic acid sequence is initiated from the promoter and results in expression of the quinone-utilizing malate dehydrogenase.

In some examples the quinone-utilizing malate dehydrogenase is characterized as EC 1.1.5.4. In some examples the quinone-utilizing malate dehydrogenase converts malate to oxaloacetate.

In some examples the quinone-utilizing malate dehydrogenase has at least 30% or higher sequence identity to one or more of the following: (1) Corynebacterium glutamicum quinone-utilizing malate dehydrogenase of SEQ ID NO: 2, (2) Escherichia coli quinone-utilizing malate dehydrogenase of SEQ ID NO: 3, (3) Helicobacter pylori quinone-utilizing malate dehydrogenase of SEQ ID NO: 4, or (4) Mycobacterium phlei quinone-utilizing malate dehydrogenase of SEQ ID NO: 5.

In some examples the quinone-utilizing malate dehydrogenase has at least 30% or higher sequence identity to Corynebacterium glutamicum quinone-utilizing malate dehydrogenase of SEQ ID NO: 2. In some of these examples the quinone-utilizing malate dehydrogenase comprises Corynebacterium glutamicum quinone-utilizing malate dehydrogenase of SEQ ID NO: 2.

In some examples the quinone-utilizing malate dehydrogenase has at least 30% or higher sequence identity to one or more of the following: (1) Solanum commersonii quinone-utilizing malate dehydrogenase of Genbank accession number JXZD01234700.1, (2) Ipomoea batatas quinone-utilizing malate dehydrogenase of Genbank accession number FLTB01001391.1, (3) Brassica oleracea quinone-utilizing malate dehydrogenase of Genbank accession number AOIX01037258.1, (4) Thlaspi arvense quinone-utilizing malate dehydrogenase of Genbank accession number AZNP01005833.1, (5) Eleusine coracana quinone-utilizing malate dehydrogenase of Genbank accession number LXGH01418531.1, (6) Tectona grandis quinone-utilizing malate dehydrogenase of Genbank accession number GFGL01159055.1, (7) Triticum urartu quinone-utilizing malate dehydrogenase of Genbank accession number AOTIO11454468.1, (8) Sesamum indicum quinone-utilizing malate dehydrogenase of Genbank accession number MB SK01001494.1, (9) Humulus lupulus quinone-utilizing malate dehydrogenase of Genbank accession number BBPC01185947.1, (10) Arachis duranensis quinone-utilizing malate dehydrogenase of Genbank accession number MAMN01020206.1, (11) Zea mays quinone-utilizing malate dehydrogenase of Genbank accession number LMVA01099495.1, (12) Corchorus olitorius quinone-utilizing malate dehydrogenase of Genbank accession number LLWS01002081.1, (13) Spinacia oleracea quinone-utilizing malate dehydrogenase of Genbank accession number AYZVO2003660.1, (14) Oryza sativa quinone-utilizing malate dehydrogenase of Genbank accession number GFYC01000193.1, (15) Ensete ventricosum quinone-utilizing malate dehydrogenase of Genbank accession number MKKS01000001.1, (16) Zea mays quinone-utilizing malate dehydrogenase of Genbank accession number OCSP01000026.1, (17) Cajanus cajan quinone-utilizing malate dehydrogenase of Genbank accession number AFSP02228873.1, (18) Coffea canephora quinone-utilizing malate dehydrogenase of Genbank accession number CBUE020014129.1, (19) Oryza sativa quinone-utilizing malate dehydrogenase of Genbank accession number AACV01031296.1, (20) Dorcoceras hygrometricum quinone-utilizing malate dehydrogenase of Genbank accession number LVEL01210429.1, (21) Ricinus communis quinone-utilizing malate dehydrogenase of Genbank accession number AASG02035827.1, (22) Arabis nordmanniana quinone-utilizing malate dehydrogenase of Genbank accession number LNCG01168830.1, (23) Suaeda salsa quinone-utilizing malate dehydrogenase of Genbank accession number GFUM01022853.1, (24) Fragaria nipponica quinone-utilizing malate dehydrogenase of Genbank accession number BATV01204972.1, (25) Pseudotsuga menziesii quinone-utilizing malate dehydrogenase of Genbank accession number LPNX010033709.1, (26) Oryza sativa quinone-utilizing malate dehydrogenase of Genbank accession number AAAA02041020.1, (27) Syzygium luehmannii quinone-utilizing malate dehydrogenase of Genbank accession number GFHM01044391.1, (28) Castanea mollissima quinone-utilizing malate dehydrogenase of Genbank accession number JRKL01150921.1, (29) Cicer arietinum quinone-utilizing malate dehydrogenase of Genbank accession number AHII02009088.1, or (30) Boehmeria nivea quinone-utilizing malate dehydrogenase of Genbank accession number NHTU01053079.1.

In some examples the promoter comprises one or more of a constitutive promoter, a seed-specific promoter, or a seed-preferred promoter.

In some examples the genetically modified plant exhibits modulated expression of the quinone-utilizing malate dehydrogenase relative to a reference plant that does not include the modified gene.

In some examples the genetically modified plant exhibits increased expression of the quinone-utilizing malate dehydrogenase relative to a reference plant that does not include the modified gene.

In some examples the genetically modified plant exhibits increased expression of the quinone-utilizing malate dehydrogenase in mitochondria of cells of the genetically modified plant relative to a reference plant that does not include the modified gene.

In some examples the modified gene further comprises a nucleic acid sequence encoding a mitochondrial targeting sequence and is further configured such that the quinone-utilizing malate dehydrogenase comprises an N-terminal mitochondrial targeting signal.

In some examples the genetically engineered plant has one or more characteristics selected from higher performance and/or seed, fruit or tuber yield relative to a reference plant that does not include the modified gene. In some of these examples the one or more characteristics are increased by 10%, 20%, 30%, 40%, 50%, 60%, 70%, 80%, 90%, 100% or higher relative to a reference plant that does not include the modified gene.

In some examples the genetically engineered plant comprises one or more of maize, wheat, oat, barley, soybean, canola, rapeseed, Brassica rapa, Brassica carinata, Brassica juncea, sunflower, safflower, oil palm, millet, sorghum, potato, lentil, chickpea, pea, pulse, bean, tomato, potato, or rice. In some examples the genetically engineered plant comprises one or more of camelina, Brassica species, Brassica napus (canola), Brassica rapa, Brassica juncea, Brassica carinata, crambe, soybean, sunflower, safflower, oil palm, flax, or cotton.

A method for producing the genetically modified plant also is disclosed. The method comprises introducing the modified gene into a plant, thereby obtaining the genetically modified plant.

### BRIEF DESCRIPTION OF THE DRAWINGS

FIG. 1 depicts the difference in the reactions of malate dehydrogenase (MDH) and malate:quinone oxidoreductase (MQO) enzymes. A. Reactions catalyzed by MDH and MQO and the thermodynamics and kinetics of the reactions. MDH catalyzed conversion of malate to oxaloacetate is thermodynamically unfavorable whereas MQO catalyzed conversion of malate to oxaloacetate is thermodynamically favorable. B. Targeting MQO to the mitochondria may accelerate the conversion of malate to oxaloacetate in the TCA cycle by alleviating the rate limitation of the MDH step in the TCA cycle.

FIG. 2 depicts a sequence alignment of malate dehydrogenase (MDH; SEQ ID NO: 1) and malate:quinone oxidoreductase (MQO; SEQ ID NO: 2) enzymes according to CLUSTAL O(1.2.4).

FIG. 3 represents a map of the plasmid vector pMBX1276 (SEQ ID NO: 51) which can be used to express the MQO encoding gene from a seed specific promoter in Camelina or canola. Plasmid pMBX1276 contains a seed-specific expression cassette for mitochondrial targeted MQO that contains the following genetic elements: the promoter from the soya bean oleosin isoform A gene; the mitochondrial targeting sequence from the gamma subunit of the mitochondrial ATP synthase from Arabidopsis thaliana; the mqo gene from Corynebacterium glutamicum codon optimized for expression in plants; and the terminator from the soya bean oleosin isoform A gene. An expression cassette for the bar gene, driven by the double enhancer CaMV 35S promoter, imparts transgenic plants resistance to the herbicide bialophos. An expression cassette for the DsRed2B gene, driven by the double enhanced CaMV 35S promoter, provides a visual marker that is used to identify transgenic seeds.

FIG. 4 details a strategy for insertion of an expression cassette for mitochondrial targeted MQO (mt-MQO) into a defined site in the plant genome through genome editing and a homologous directed repair mechanism. An sgRNA with a guide sequence for the genomic location of interest (for example Guide #1) is used to enable the Cas enzyme, or other CRISPR nuclease, to produce a double stranded break in the genome. An expression cassette containing a seed specific promoter, the mt-MQO gene, and an appropriate 3′ UTR sequence is flanked by sequences with homology to the upstream and downstream region of the sgRNA cut site. This expression cassette is inserted into the double stranded break in genomic DNA using the homology directed repair mechanism of the plant.

FIG. 5 illustrates the T-DNA insert expected from transformation of maize with a binary construct containing an expression cassette for MQO targeted to the mitochondria of seeds. The T-DNA insert contains a maize trpA promoter (SEQ ID NO: 41); an N-terminal mitochondrial targeting sequence from the Arabidopsis F-ATPase gamma subunit codon optimized for maize; the mqo gene from Corynebacterium glutamicum codon optimized for maize; and the PINII termination sequence.

### DETAILED DESCRIPTION OF THE INVENTION

Plant cells, tissues and plants with modulated expression, preferably increased expression of MQO genes are disclosed. In preferred embodiments, the plant cells, tissues and plants comprise increased expression of quinone-utilizing malate dehydrogenase (Mqo; EC 1.1.5.4) genes such that the rate of conversion of malate to OAA in the mitochondria is increased resulting in increased crop performance and/or yield. The genes encoding the MQO enzyme can be used alone or in combination with altered expression of additional genes to enhance photosynthesis or carbon partitioning to seed. The expression of the genes encoding the MQO proteins can be increased using genetic engineering techniques or marker assisted breeding approaches to develop plants with increased performance and/or yield. Where genetic engineering techniques are used to increase the expression of the MQO proteins, the increased expression can be accomplished using transgenic technologies. The MQO genes can be expressed and the MQO proteins targeted to the plant mitochondria alone or in combinations with mitochondrial transporters or other genes described herein. For example the MQO gene can be used alone or in combinations with the CCP1 like mitochondrial transporters from algal or plant sources which have been shown to reduce photorespiration/respiration and increase crop yield. For example, it has recently been shown by Schnell et al., WO 2015/103074 that Camelina plants transformed to express CCP1 gene of the algal species Chlamydomonas reinhardtii have reduced transpiration rates, increased CO₂ assimilation rates and higher yield than control plants which do not express the CCP1 gene.

In Patent Application PCT/US2017/016421, to Yield10 Bioscience, a number of orthologs of CCP1 from algal species that share common protein sequence domains including mitochondrial membrane domains and transporter protein domains were shown to increase seed yield and reduce seed size when expressed constitutively in Camelina plants. Schnell et al., WO 2015/103074, also reported a decrease in seed size in higher yielding Camelina lines expressing CCP1.

In Patent Application PCT/US2018/019105, to Yield10 Bioscience, CCP1 and its orthologs from other eukaryotic algae are referred to as mitochondrial transporter proteins. The inventors tested the impact of expressing CCP1 or its algal orthologs using seed-specific promoters with the unexpected outcome that both seed yield and seed size increased. These inventors also recognized the benefits of combining constitutive expression and seed specific expression of CCP1 or any of its orthologs in the same plant.

In Patent Application PCT/US2018/037740, to Yield10 Bioscience, sequence and structural orthologs of CCP1 were identified in a select number of plant species for the first time and the inventors disclosed genetically engineered land plants that express plant CCP1-like mitochondrial transporter proteins.

In Patent Application PCT/US2018/038927, to Yield10 Bioscience, methods, genes and systems for producing land plants with increased expression of plant plastidial dicarboxylate transporter genes and proteins is described.

The Chlamydomonas reinhardtii CCP1, when genetically engineered into plants, is thought to facilitate malate/OAA transfer in and out of the mitochondrion resulting in increases in seed fruit or tuber yield.

In general, the key elements of crop yield and in particular seed, fruit or tuber yield can be divided into two parts: photosynthetic carbon capture to produce sucrose in the green tissue is referred to as the carbon source; followed by the transfer of carbon in the form of sucrose to the developing seed, fruit or tuber tissue which is referred to as the carbon sink. The flow of carbon from source tissue to sink tissue is subject to complex regulatory mechanisms. Increasing the seed fruit or tuber yield of a given crop is therefore dependent not only on improving photosynthetic efficiency in the source tissue but also increasing the strength of the sink tissue to pull fixed carbon into the development of seeds, fruit or tubers. Sink strength is in turn dependent on the metabolic processes taking place there and in particular the tricarboxylic acid cycle (TCA cycle) which provides metabolic building blocks as well as energy for seed, fruit or tuber biosynthesis.

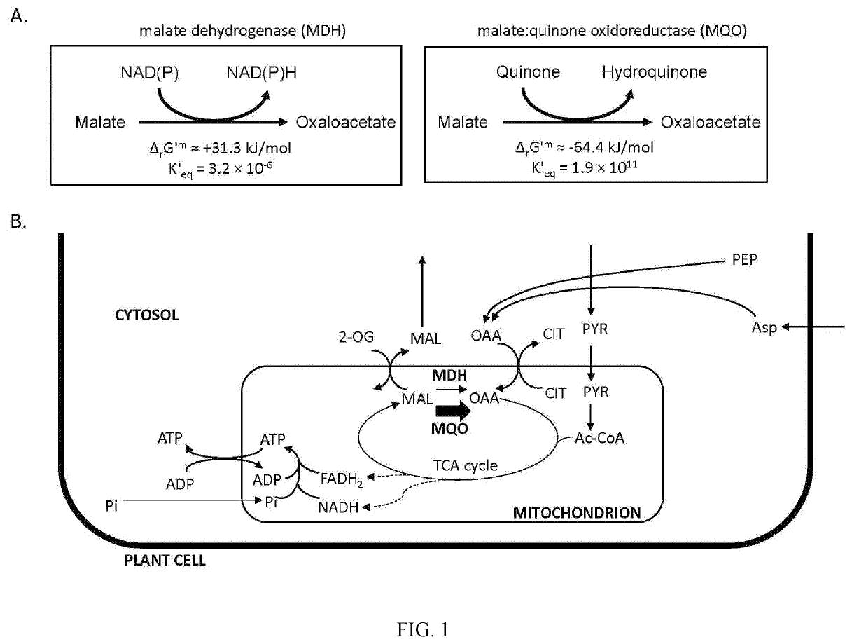

In the metabolism within developing seeds, the TCA cycle is expected to operate in mitochondria to provide energy in the form of NADH and ATP from sugar metabolism. In which case, malate must be converted to oxaloacetate by malate dehydrogenase (MDH). Plants typically contain only NAD(P)H-dependent MDHs, and these enzymes catalyze reactions with thermodynamics that greatly favor malate formation (FIG. 1), even when the [NAD⁺]/[NADH] ratio is high. MDHs with NAD(P)H as cofactor are soluble enzymes and thus are not linked directly to respiration as is the case with for example succinate dehydrogenase, which can proceed in an unfavorable thermodynamic direction with ease because it is coupled to a reaction (donation of electrons to oxygen) that is extremely favorable, making the overall thermodynamics actually favor electron donation.

Experimental metabolic flux analysis data show that despite its unfavorable thermodynamics, often a flux from malate to oxaloacetate in seed mitochondria still apparently occurs [see, e.g., V. V. Iyer, G. Sriram, D. B. Fulton, R. Zhou, M. E. Westgate, and J. V. Shanks, Metabolic flux maps comparing the effect of temperature on protein and oil biosynthesis in developing soybean cotyledons, Plant, Cell and Environment 31:506-517 (2008); D. K. Allen, J. B. Ohlrogge, and Y. Shachar-Hill, The role of light in soybean seed filling metabolism, Plant J. 58:220-234 (2009); G. Sriram, D. B. Fulton, V. V. Iyer, J. M. Peterson, R. Zhou, M. E. Westgate, M. H. Spalding, and J. V. Shanks, Quantification of compartmented metabolic fluxes in developing soybean embryos by employing biosynthetically directed fractional ¹³C labeling, two-dimensional [¹³C, ¹H] nuclear magnetic resonance, and comprehensive isotopomer balancing, Plant Physiol. 136:3043-3057 (2004); A. P. Alonso, F. D. Goffman, J. B. Ohlrogge, and Y. Shachar-Hill, Carbon conversion efficiency and central metabolic fluxes in developing sunflower (Helianthus annuus L.) embryos, Plant J. 52:296-308 (2007)]. These data do not typically show the action of a di- or tricarboxylate transporter interrupting this flux, suggesting that these transporters are often not significantly active in seed tissue. This analysis points to malate dehydrogenase as a possible rate limiting step in plant mitochondrial metabolism.

A potential solution to the rate limitation at the malate dehydrogenase step of the TCA cycle in mitochondria, would be to increase the expression of a malate dehydrogenase that is associated with a more thermodynamically favorable electron acceptor than NAD(P)⁺, such as the quinone-utilizing variety (Mqo; EC 1.1.5.4; FIG. 1). Bacteria routinely use Mqo for malate oxidation to complete the TCA cycle even though the resulting electrons are ultimately lost to oxygen rather than being harnessed for biosynthesis. Bacteria, unlike plants, are subject to fierce competition for carbon sources, and thus it is often more advantageous for them to utilize carbon quickly than to use carbon particularly efficiently. The increased expression of the Mqo in the mitochondria provides a means to improve crop performance and/or seed, fruit or tuber yield.

Several studies suggest that the yield of sink tissue such as seed, fruit or tubers is limited under some circumstances by the strength of the sink demand. In this sense, kinetic improvement of the TCA cycle via Mqo, even though it would not necessarily increase carbon efficiency, could induce higher overall photosynthate production at source tissues such as leaves which would lead to an overall improvement in sink-tissue (seed, fruit or tuber) production.

### MQO Proteins and Genes

Mqo is a bacterial membrane-associated enzyme, and so expression in higher-plant seed mitochondria could be subject to incompatibilities in electron acceptor or membrane positioning. Mqo is not an integral membrane protein, but rather peripherally associates with the membrane (D. Molenaar, et. al., Biochemical and genetic characterization of the membrane-associated malate dehydrogenase (acceptor) from Corynebacterium glutamicum, Eur. J. Biochem. 254: 395-403 (1998)), making it more likely to be compatible with diverse membrane types. It is also known to utilize many different electron acceptors (D. Molenaar, et. al., (1998), and thus those already present in the plant mitochondrion may be sufficient for its operation.

The Mqo enzymes from a few bacterial species have been characterized: Corynebacterium glutamicum (D. Molenaar, M. E. van der Rest, A. Drysch, and R. Yücel, Functions of the membrane-associated and cytoplasmic malate dehydrogenases in the citric acid cycle of Corynebacterium glutamicum, J. Bacteriol. 182:6884-6891 (2000); D. Molenaar, et al., (1998)), Escherichia coli (M. E. van der Rest, et. al., Functions of the membrane-associated and cytoplasmic malate dehydrogenases in the citric acid cycle of Escherichia coli, J. Bacteriol. 182:6892-6899 (2000)), Helicobacter pylori (B. Kather, et. al., Another type of citric acid cycle enzyme in Helicobacter pylori: the malate: quinone oxidoreductase, J. Bacteriol. 182:3204-3209 (2000)), Bacillus sp. DSM 465 (T. Ohshima and S. Tanaka, Dye-linked L-malate dehydrogenase from thermophilic Bacillus species DSM 465: purification and characterization, Eur. J. Biochem. 214:37-42 (1993), Mycobacterium sp. (T. Imai, FAD-dependent malate dehydrogenase, a phospholipid-requiring enzyme from Mycobacterium sp. strain Takeo: Purification and some properties, Biochim. Biophys. Acta 523:37-46 (1978)), and Mycobacterium phlei (K. Imai and A. F. Brodie, A phospholipid-requiring enzyme, malate-vitamin K reductase, J. Biol. Chem. 248:7487-7494 (1973)).

Corynebacterium glutamicum relies on Mqo for a functional TCA cycle; Mqo mutants have difficulty growing on minimal medium containing glucose, mannitol, or acetate, whereas MDH mutants have no discernible phenotype (D. Molenaar, et al., (2000)). When Corynebacterium MDH is purified and incubated together with isolated Corynebacterium membranes, the net reaction is oxidation of NADH and reduction of oxaloacetate, indicating that the predicted thermodynamics for these enzymes are generally correct (D. Molenaar, et. al., (1998)). Furthermore, purified Corynebacterium MDH reduces oxaloacetate readily but does not oxidize malate effectively, even when conditions are biased in its favor (D. Molenaar, et. al., (1998)).

In Escherichia coli, the loss of Mqo does not result in an observable growth phenotype, while the loss of MDH does, suggesting that malate could be oxidized by MDH in this organism to some degree, though high malate concentrations would probably be required to do this. However, even an Mqo MDH double mutant still grows on some carbon sources, suggesting that Escherichia coli possesses an alternative route from malate to oxaloacetate in practice (M. E. van der Rest, et. al., (2000)).

Helicobacter pylori must use Mqo for direct malate oxidation, because it does not contain a gene encoding an MDH (B. Kather, et. al., (2000)).

In order to express a protein in the mitochondria in a higher plant, the gene should be modified to include a mitochondrial targeting sequence operably linked to the gene and integrated into nuclear DNA (R. S. Allen, K. Tilbrook, A. C. Warden, P. C. Campbell, V. Rolland, S. P. Singh, and C. C. Wood, Expression of 16 nitrogenase proteins within the plant mitochondrial matrix, Front. Plant Sci. 8:287-300 (2017); S. Lee, D. W. Lee, Y. J. Yoo, O. Duncan, Y. J. Oh, Y. J. Lee, G. Lee, J. Whelan, and I. Hwang, Mitochondrial targeting of the Arabidopsis F1-ATPase γ-subunit via multiple compensatory and synergistic presequence motifs, Plant Cell 24:5037-5057 (2012)).

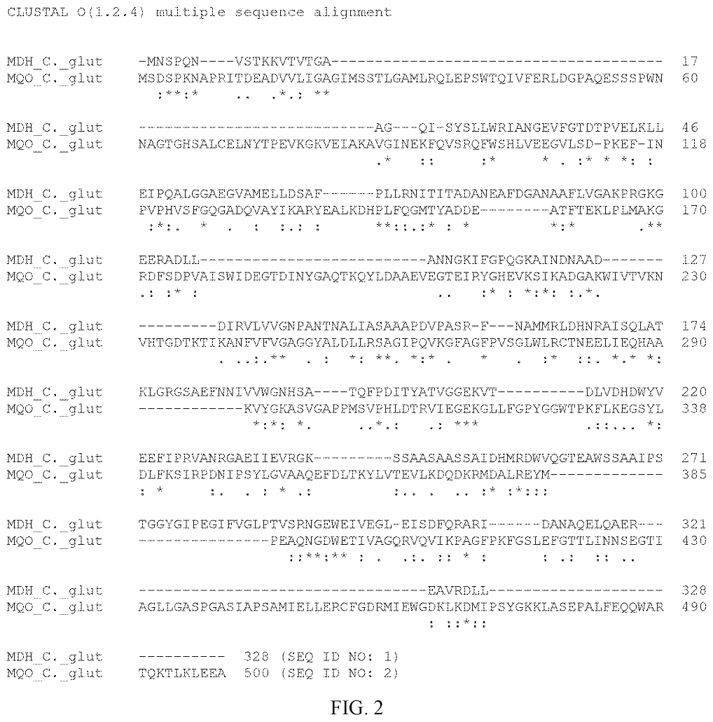

Numerous bacterial examples of Mqo are known. The examples mentioned in the text for which sequences are known are listed in TABLE 1, though this is meant to be illustrative of the many possible sources and by no means an exhaustive list. A BLAST search using the Corynebacterium glutamicum Mqo protein sequence was performed to find Mqo homologs in higher plants. Both blastp and tblastn searches at the NCBI BLAST website (website: blast.ncbi.nlm.nih.gov/Blast.cgi) were performed for green plants using the nr, TSA, and wgs databases. It should be noted that the Corynebacterium glutamicum MDH protein (SEQ ID NO: 1) is quite dissimilar from its Mqo protein (SEQ ID NO: 2; FIG. 2), and therefore BLAST hits to Mqo are not likely to be MDH proteins. The best hit from each distinct plant species is listed in TABLE 2. This listing also is illustrative but by no means exhaustive.

TABLE 1
Examples of bacterial Mqo proteins.

| | | GenBank | SEQ ID |
|---|---|---|---|
| Organism | Locus | accession | NO: |
| Corynebacterium | MQO_CORGL | O69282.3 | 2 |
| glutamicum | | | |
| Escherichia coli | MQO_ECOLI | P33940.2 | 3 |
| Helicobacter pylori | MQO_HELPY | O24913.1 | 4 |
| Mycobacterium phlei | WP_081491246 | WP_081491246.1 | 5 |

TABLE 2
BLAST hits of Corynebacterium glutamicum Mqo from higher plants.

| Organism/Description | E value | GenBank accession |
|---|---|---|
| Solanum commersonii cultivar cmm1t C2859530_1, whole | 1.00E−166 | JXZD01234700.1 |
| genome shotgun sequence | | |
| Ipomoea batatas genome assembly, contig: SP3_ctg79568, | 3.00E−161 | FLTB01001391.1 |
| whole genome shotgun sequence | | |
| Brassica oleracea var. capitata cultivar line 02-12 | 1.00E−160 | AOIX01037258.1 |
| Scaffold001235_2, whole genome shotgun sequence | | |
| Thlaspi arvense cultivar MN106 Ta_scaffold_5838, whole | 2.00E−158 | AZNP01005833.1 |
| genome shotgun sequence | | |
| Eleusine coracana subsp. coracana cultivar ML-365 | 1.00E−152 | LXGH01418531.1 |
| scaffold74750, whole genome shotgun sequence | | |
| TSA: Tectona grandis TR49592_c1_g1_i2 transcribed RNA | 2.00E−152 | GFGL01159055.1 |
| sequence | | |
| Triticum urartu cultivar G1812 contig1454469, whole | 2.00E−151 | AOTI011454468.1 |
| genome shotgun sequence | | |
| Sesamum indicum isolate Yuzhi11 scaffold02289, whole | 2.00E−151 | MBSK01001494.1 |
| genome shotgun sequence | | |
| Humulus lupulus var. lupulus DNA, contig: | 9.00E−146 | BBPC01185947.1 |
| SW_scaffold49042_size12079_1, whole genome shotgun | | |
| sequence | | |
| Arachis duranensis cultivar PI475845 scaffold5783, whole | 5.00E−145 | MAMN01020206.1 |
| genome shotgun sequence | | |
| Zea mays subsp. mexicana cultivar TEO scaffold99552, | 1.00E−142 | LMVA01099495.1 |
| whole genome shotgun sequence | | |
| Corchorus olitorius cultivar JRO-524 Co_S7_contig02240, | 9.00E−141 | LLWS01002081.1 |
| whole genome shotgun sequence | | |
| Spinacia oleracea cultivar SynViroflay | 2.00E−140 | AYZV02003660.1 |
| scaffold776.con0110.1, whole genome shotgun sequence | | |
| TSA: Oryza sativa tig00001037_pilon transcribed RNA | 5.00E−140 | GFYC01000193.1 |
| sequence | | |
| Ensete ventricosum cultivar Derea scf_29696_1.contig_1, | 2.00E−139 | MKKS01000001.1 |
| whole genome shotgun sequence | | |
| Zea mays subsp. mays genome assembly, contig: | 5.00E−136 | OCSP01000026.1 |
| oilsands_bin_084_25, whole genome shotgun sequence | | |
| Cajanus cajan strain Asha PairedContig_245384, whole | 1.00E−134 | AFSP02228873.1 |
| genome shotgun sequence | | |
| Coffea canephora WGS project CBUE00000000 data, strain | 1.00E−132 | CBUE020014129.1 |
| DH200-94, contig contig11088, whole genome shotgun | | |
| sequence | | |
| Oryza sativa Japonica Group cultivar Nipponbare | 3.00E−101 | AACV01031296.1 |
| Ctg031296, whole genome shotgun sequence | | |
| Dorcoceras hygrometricum cultivar XS01 contig210429, | 3.00E−83 | LVEL01210429.1 |
| whole genome shotgun sequence | | |
| Ricinus communis cultivar Hale ctg_1100012333500, whole | 1.00E−78 | AASG02035827.1 |
| genome shotgun sequence | | |
| Arabis nordmanniana contig_26516, whole genome shotgun | 3.00E−77 | LNCG01168830.1 |
| sequence | | |
| TSA: Suaeda salsa c181558.graph_c0 transcribed RNA | 5.00E−72 | GFUM01022853.1 |
| sequence | | |
| Fragaria nipponica DNA, contig: FNI_icon04402893.1, | 1.00E−71 | BATV01204972.1 |
| whole genome shotgun sequence | | |
| Pseudotsuga menziesii isolate Weyco1 jcf7190000448181, | 2.00E−66 | LPNX010033709.1 |
| whole genome shotgun sequence | | |
| Oryza sativa Indica Group cultivar 93-11 Ctg041020, whole | 4.00E−62 | AAAA02041020.1 |
| genome shotgun sequence | | |
| TSA: Syzygium luehmannii TRINITY_DN69857_c0_g1_i1 | 4.00E−60 | GFHM01044391.1 |
| transcribed RNA sequence | | |
| Castanea mollissima cultivar Vanuxem contig236763, | 1.00E−58 | JRKL01150921.1 |
| whole genome shotgun sequence | | |
| Cicer arietinum cultivar ICC4958 scaffold17755, whole | 4.00E−57 | AHII02009088.1 |
| genome shotgun sequence | | |
| Boehmeria nivea cultivar ZZ1 scaffold109813, whole | 1.00E−54 | NHTU01053079.1 |
| genome shotgun sequence | | |

MQO genes from any source can be used but in most cases it is preferable for the plant to be genetically engineered to increase expression of the MQO proteins in the mitochondria of the plant cells. Accordingly, disclosed herein is a genetically engineered plant having increased expression of one or more MQO proteins. Preferably the genetically engineered plant described herein has increased expression of one or more MQO proteins in the mitochondria and has higher performance, seed, fruit or tuber yield. In a preferred embodiment the expression of the MQO protein is directed from a plant seed specific or seed-preferred promoter.

Accordingly, provided herein are methods and compositions for modifying a plant, the method comprising modulating or more preferably increasing the expression of

(a) one or more MQO polynucleotides or polypeptides as listed in TABLE 1 or TABLE 2; or

(b) one or more polynucleotides or polypeptides comprising or consisting of a sequence having at least 30%, 35%, 40%, 45%, 50%, 55%, 60%, 65%, 70%, 75%, 80%, 85%, 90%, 95% or higher sequence identity to one or more MQO polynucleotides or polypeptides as listed in TABLE 1 or TABLE 2.

Thus, as noted above, a genetically engineered plant that expresses a quinone-utilizing malate dehydrogenase is disclosed.

In some examples the quinone-utilizing malate dehydrogenase is characterized as EC 1.1.5.4. In some examples the quinone-utilizing malate dehydrogenase converts malate to oxaloacetate.

In some examples the quinone-utilizing malate dehydrogenase has at least 30% or higher sequence identity to one or more of the following: (1) Corynebacterium glutamicum quinone-utilizing malate dehydrogenase of SEQ ID NO: 2, (2) Escherichia coli quinone-utilizing malate dehydrogenase of SEQ ID NO: 3, (3) Helicobacter pylori quinone-utilizing malate dehydrogenase of SEQ ID NO: 4, or (4) Mycobacterium phlei quinone-utilizing malate dehydrogenase of SEQ ID NO: 5. For example, the quinone-utilizing malate dehydrogenase can have at least 35%, 40%, 45%, 50%, 55%, 60%, 65%, 70%, 75%, 80%, 85%, 90%, 95% or higher sequence identity to one or more these quinone-utilizing malate dehydrogenases.

In some examples the quinone-utilizing malate dehydrogenase has at least 30% or higher sequence identity to Corynebacterium glutamicum quinone-utilizing malate dehydrogenase of SEQ ID NO: 2. For example, the quinone-utilizing malate dehydrogenase can have at least 35%, 40%, 45%, 50%, 55%, 60%, 65%, 70%, 75%, 80%, 85%, 90%, 95% or higher sequence identity to Corynebacterium glutamicum quinone-utilizing malate dehydrogenase of SEQ ID NO: 2. In some of these examples the quinone-utilizing malate dehydrogenase comprises Corynebacterium glutamicum quinone-utilizing malate dehydrogenase of SEQ ID NO: 2.

In some examples the quinone-utilizing malate dehydrogenase has at least 30% or higher sequence identity to one or more of the following: (1) Solanum commersonii quinone-utilizing malate dehydrogenase of Genbank accession number JXZD01234700.1, (2) Ipomoea batatas quinone-utilizing malate dehydrogenase of Genbank accession number FLTB01001391.1, (3) Brassica oleracea quinone-utilizing malate dehydrogenase of Genbank accession number AOIX01037258.1, (4) Thlaspi arvense quinone-utilizing malate dehydrogenase of Genbank accession number AZNP01005833.1, (5) Eleusine coracana quinone-utilizing malate dehydrogenase of Genbank accession number LXGH01418531.1, (6) Tectona grandis quinone-utilizing malate dehydrogenase of Genbank accession number GFGL01159055.1, (7) Triticum urartu quinone-utilizing malate dehydrogenase of Genbank accession number AOTIO11454468.1, (8) Sesamum indicum quinone-utilizing malate dehydrogenase of Genbank accession number MBSK01001494.1, (9) Humulus lupulus quinone-utilizing malate dehydrogenase of Genbank accession number BBPC01185947.1, (10) Arachis duranensis quinone-utilizing malate dehydrogenase of Genbank accession number MAMN01020206.1, (11) Zea mays quinone-utilizing malate dehydrogenase of Genbank accession number LMVA01099495.1, (12) Corchorus olitorius quinone-utilizing malate dehydrogenase of Genbank accession number LLWS01002081.1, (13) Spinacia oleracea quinone-utilizing malate dehydrogenase of Genbank accession number AYZVO2003660.1, (14) Oryza sativa quinone-utilizing malate dehydrogenase of Genbank accession number GFYC01000193.1, (15) Ensete ventricosum quinone-utilizing malate dehydrogenase of Genbank accession number MKKS01000001.1, (16) Zea mays quinone-utilizing malate dehydrogenase of Genbank accession number OCSP01000026.1, (17) Cajanus cajan quinone-utilizing malate dehydrogenase of Genbank accession number AFSP02228873.1, (18) Coffea canephora quinone-utilizing malate dehydrogenase of Genbank accession number CBUE020014129.1, (19) Oryza sativa quinone-utilizing malate dehydrogenase of Genbank accession number AACV01031296.1, (20) Dorcoceras hygrometricum quinone-utilizing malate dehydrogenase of Genbank accession number LVEL01210429.1, (21) Ricinus communis quinone-utilizing malate dehydrogenase of Genbank accession number AASG02035827.1, (22) Arabis nordmanniana quinone-utilizing malate dehydrogenase of Genbank accession number LNCG01168830.1, (23) Suaeda salsa quinone-utilizing malate dehydrogenase of Genbank accession number GFUM01022853.1, (24) Fragaria nipponica quinone-utilizing malate dehydrogenase of Genbank accession number BATV01204972.1, (25) Pseudotsuga menziesii quinone-utilizing malate dehydrogenase of Genbank accession number LPNX010033709.1, (26) Oryza sativa quinone-utilizing malate dehydrogenase of Genbank accession number AAAA02041020.1, (27) Syzygium luehmannii quinone-utilizing malate dehydrogenase of Genbank accession number GFHM01044391.1, (28) Castanea mollissima quinone-utilizing malate dehydrogenase of Genbank accession number JRKL01150921.1, (29) Cicer arietinum quinone-utilizing malate dehydrogenase of Genbank accession number AHII02009088.1, or (30) Boehmeria nivea quinone-utilizing malate dehydrogenase of Genbank accession number NHTU01053079.1. For example, the quinone-utilizing malate dehydrogenase can have at least 35%, 40%, 45%, 50%, 55%, 60%, 65%, 70%, 75%, 80%, 85%, 90%, 95% or higher sequence identity to one or more these quinone-utilizing malate dehydrogenases.

The genetically engineered plant comprises a modified gene for the quinone-utilizing malate dehydrogenase. The modified gene comprises (i) a promoter and (ii) a nucleic acid sequence encoding the quinone-utilizing malate dehydrogenase.

The promoter is non-cognate with respect to the nucleic acid sequence encoding the quinone-utilizing malate dehydrogenase. A promoter that is non-cognate with respect to a nucleic acid sequence means that the promoter is not naturally paired with the nucleic acid sequence in organisms from which the promoter and/or the nucleic acid sequence are derived. Instead, the promoter has been paired with the nucleic acid sequence based on use of recombinant DNA techniques to create a modified gene.

The modified gene is configured such that transcription of the nucleic acid sequence is initiated from the promoter and results in expression of the quinone-utilizing malate dehydrogenase. Accordingly, in the context of the modified gene, the promoter functions as a promoter of transcription of the nucleic acid sequence, and thus of expression of the the quinone-utilizing malate dehydrogenase. In preferred examples, the expression of the the quinone-utilizing malate dehydrogenase is higher in the genetically engineered land plant than in a corresponding plant that does not include the modified gene.

In some examples the promoter comprises one or more of a constitutive promoter, a seed-specific promoter, or a seed-preferred promoter. Suitable promoters are discussed below.

In some examples the genetically modified plant exhibits increased expression of the quinone-utilizing malate dehydrogenase in mitochondria of cells of the genetically modified plant relative to a reference plant that does not include the modified gene.

In some examples the modified gene further comprises a nucleic acid sequence encoding a mitochondrial targeting sequence and is further configured such that the quinone-utilizing malate dehydrogenase comprises an N-terminal mitochondrial targeting signal.

### Plants

A “plant,” as the term is used herein, generally refers to a plant belonging to the plant subkingdom Embryophyta, including higher plants, also termed vascular plants, and mosses, liverworts, and hornworts.

The term “plant” includes mature plants, seeds, shoots and seedlings, and parts, propagation material, plant organ tissue, protoplasts, callus and other cultures, for example cell cultures, derived from plants belonging to the plant subkingdom Embryophyta, and all other species of groups of plant cells giving functional or structural units, also belonging to the plant subkingdom Embryophyta. The term “mature plants” refers to plants at any developmental stage beyond the seedling. The term “seedlings” refers to young, immature plants at an early developmental stage.

Plants encompass all annual and perennial monocotyledonous or dicotyledonous plants and includes by way of example, but not by limitation, those of the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea, Populus, Camelina, Beta, Solanum, and Carthamus. Preferred plants are those from the following plant families: Amaranthaceae, Asteraceae, Brassicaceae, Carophyllaceae, Chenopodiaceae, Compositae, Cruciferae, Cucurbitaceae, Euphorbiaceae, Fabaceae, Labiatae, Leguminosae, Papilionoideae, Liliaceae, Linaceae, Malvaceae, Poaceae, Rosaceae, Rubiaceae, Saxifragaceae, Scrophulariaceae, Solanaceae, Sterculiaceae, Tetragoniaceae, Theaceae, Umbelliferae.

The plant can be a monocotyledonous plant or a dicotyledonous plant. Preferred dicotyledonous plants are selected in particular from the dicotyledonous crop plants such as, for example, Asteraceae such as sunflower, tagetes or calendula and others; Compositae, especially the genus Lactuca, very particularly the species sativa (lettuce) and others; Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli) and other cabbages; and the genus Arabidopsis, very particularly the species thaliana, and cress or canola and others; Cucurbitaceae such as melon, pumpkin/squash or zucchini and others; Leguminosae, particularly the genus Glycine, very particularly the species max (soybean), soya, and alfalfa, pea, beans or peanut and others; Rubiaceae, preferably the subclass Lamiidae such as, for example Coffea arabica or Coffea liberica (coffee bush) and others; Solanaceae, particularly the genus Lycopersicon, very particularly the species esculentum (tomato), the genus Solanum, very particularly the species tuberosum (potato) and melongena (aubergine) and the genus Capsicum, very particularly the genus annuum (pepper) and tobacco or paprika and others; Sterculiaceae, preferably the subclass Dilleniidae such as, for example, Theobroma cacao (cacao bush) and others; Theaceae, preferably the subclass Dilleniidae such as, for example, Camellia sinensis or Thea sinensis (tea shrub) and others; Umbelliferae, particularly the genus Daucus (very particularly the species carota (carrot)) and Apium (very particularly the species graveolens dulce (celery)) and others; and linseed, cotton, hemp, flax, cucumber, spinach, carrot, sugar beet and the various tree, nut and grapevine species, in particular banana and kiwi fruit. Preferred monocotyledonous plants include maize, rice, wheat, sugarcane, sorghum, oats and barley.

Oil crops encompass by way of example: Borago officinalis (borage); Camelina (false flax); Brassica species such as B. campestris, B. napus, B. rapa, B. carinata (mustard, oilseed rape or turnip rape); Cannabis sativa (hemp); Carthamus tinctorius (safflower); Cocos nucifera (coconut); Crambe abyssinica (crambe); Cuphea species (Cuphea species yield fatty acids of medium chain length, in particular for industrial applications); Elaeis guinensis (African oil palm); Elaeis oleifera (American oil palm); Glycine max (soybean); Gossypium hirsutum (American cotton); Gossypium barbadense (Egyptian cotton); Gossypium herbaceum (Asian cotton); Helianthus annuus (sunflower); Jatropha curcas (jatropha); Linum usitatissimum (linseed or flax); Oenothera biennis (evening primrose); Olea europaea (olive); Oryza sativa (rice); Ricinus communis (castor); Sesamum indicum (sesame); Thlaspi caerulescens (pennycress); Triticum species (wheat); Zea mays (maize), and various nut species such as, for example, walnut or almond.

Camelina species, commonly known as false flax, are native to Mediterranean regions of Europe and Asia and seem to be particularly adapted to cold semiarid climate zones (steppes and prairies). The species Camelina sativa was historically cultivated as an oilseed crop to produce vegetable oil and animal feed. In addition to being useful as an industrial oilseed crop, Camelina is a very useful model system for developing new tools and genetically engineered approaches to enhancing the yield of crops in general and for enhancing the yield of seed and seed oil in particular. Demonstrated transgene improvements in Camelina can then be deployed in major oilseed crops including Brassica species including B. napus (canola), B. rapa, B. juncea, B. carinata, crambe, soybean, sunflower, safflower, oil palm, flax, and cotton.

As will be apparent, the plant can be a C3 photosynthesis plant, i.e. a plant in which RubisCO catalyzes carboxylation of ribulose-1,5-bisphosphate by use of CO₂ drawn directly from the atmosphere, such as for example, wheat, oat, and barley, among others. The plant also can be a C4 plant, i.e. a plant in which RubisCO catalyzes carboxylation of ribulose-1,5-bisphosphate by use of CO₂ shuttled via malate or aspartate from mesophyll cells to bundle sheath cells, such as for example maize, millet, and sorghum, among others.

Accordingly, in some examples the genetically engineered plant is a C3 plant. Also, in some examples the genetically engineered plant is a C4 plant. Also, in some examples the genetically engineered plant is a major food or feed crop plant selected from the group consisting of maize, wheat, oats, barley, soybean, canola, rapeseed, Brassica rapa, Brassica carinata, Brassica juncea, sunflower, safflower, oil palm, millet, sorghum, potato, lentils, chickpeas, peas, pulses, beans, tomato, potato and rice. In some of these examples, the genetically engineered plant is maize. Also, in some examples the genetically engineered plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

Thus, in some examples the genetically engineered plant comprises one or more of maize, wheat, oat, barley, soybean, canola, rapeseed, Brassica rapa, Brassica carinata, Brassica juncea, sunflower, safflower, oil palm, millet, sorghum, potato, lentil, chickpea, pea, pulse, bean, tomato, potato, or rice. In some examples the genetically engineered plant comprises one or more of camelina, Brassica species, Brassica napus (canola), Brassica rapa, Brassica juncea, Brassica carinata, crambe, soybean, sunflower, safflower, oil palm, flax, or cotton.

Modulated and/or Increased Expression of MQO Proteins

As noted above, in some examples the genetically modified plant exhibits modulated expression of the quinone-utilizing malate dehydrogenase relative to a reference plant that does not include the modified gene. In some examples the genetically modified plant exhibits increased expression of the quinone-utilizing malate dehydrogenase relative to a reference plant that does not include the modified gene.

In certain embodiments, the genetically engineered plant having increased expression of one or more MQO proteins can have a CO₂ assimilation rate that is higher than for a corresponding reference plant not having the increased expression of one or more MQO proteins. For example, the genetically engineered plant can have a CO₂ assimilation rate that is at least 5% higher, at least 10% higher, at least 20% higher, or at least 40% higher, than for a corresponding reference plant that does not have the increased expression of one or more MQO proteins.

The genetically engineered plant having increased expression of one or more MQO proteins also can have a seed, fruit or tuber yield that is higher than for a corresponding reference plant not having the increased expression of one or more MQO proteins. For example, the genetically engineered plant can have a seed yield that is at least 5% higher, at least 10% higher, at least 20% higher, at least 40% higher, at least 60% higher, or at least 80% higher, than for a corresponding reference plant that does not have the increased expression of one or more MQO proteins.

The genetically engineered plant having increased expression of one or more MQO proteins also can produce larger seeds, fruits or tubers than a corresponding reference plant not having the increased expression of one or more MQO proteins. For example, the genetically engineered plant can produce seeds, fruits or tubers that are at least 5% larger, at least 10% larger, at least 20% larger, at least 40% larger, at least 60% larger, or at least 80% larger, than for a corresponding reference plant that does not have the increased expression of one or more MQO proteins.

The genetically engineered plant having increased expression of one or more MQO proteins can also produce an increased number of seeds, fruits or tubers than a corresponding reference plant not having the increased expression of one or more MQO proteins. For example, the genetically engineered plant can produce a number of seeds, fruits or tubers that is at least 5% higher, at least 10% higher, at least 20% higher, at least 40% higher, at least 60% higher, or at least 80% higher, than for a corresponding reference plant that does not have the increased expression of one or more MQO proteins.

Thus, in some examples the genetically engineered plant has one or more characteristics selected from higher performance and/or seed, fruit or tuber yield relative to a reference plant that does not include the modified gene. In some of these examples the one or more characteristics are increased by 10%, 20%, 30%, 40%, 50%, 60%, 70%, 80%, 90%, 100% or higher relative to a reference plant that does not include the modified gene.

### Methods of Making the Genetically Engineered Plant

As noted above, a method for producing the genetically modified plant also is disclosed. The method comprises introducing the modified gene into a plant, thereby obtaining the genetically modified plant.

Following identification of suitable MQO proteins, a genetically engineered plant having increased expression of the one or more MQO proteins in the mitochondria can be made by methods that are known in the art, for example as follows.

DNA constructs useful in the methods described herein include transformation vectors capable of introducing transgenes or other modified nucleic acid sequences into plants. As used herein, “genetically engineered” refers to an organism in which a nucleic acid fragment containing a heterologous nucleotide sequence has been introduced, or in which the expression of a homologous gene has been modified, for example by genome editing. Transgenes in the genetically engineered organism are preferably stable and inheritable. Heterologous nucleic acid fragments may or may not be integrated into the host genome.

Several plant transformation vector options are available, including those described in Gene Transfer to Plants, 1995, Potrykus et al., eds., Springer-Verlag Berlin Heidelberg New York, Genetically engineered Plants: A Production System for Industrial and Pharmaceutical Proteins, 1996, Owen et al., eds., John Wiley & Sons Ltd. England, and Methods in Plant Molecular Biology: A Laboratory Course Manual, 1995, Maliga et al., eds., Cold Spring Laboratory Press, New York. Plant transformation vectors generally include one or more coding sequences of interest under the transcriptional control of 5′ and 3′ regulatory sequences, including a promoter, a transcription termination and/or polyadenylation signal, and a selectable or screenable marker gene.

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB 10 and hygromycin selection derivatives thereof. See, for example, U.S. Pat. No. 5,639,949.

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. The choice of vector for transformation techniques that do not rely on Agrobacterium depends largely on the preferred selection for the species being transformed. Typical vectors suitable for non-Agrobacterium transformation include pCIB3064, pSOG 19, and pSOG35. See, for example, U.S. Pat. No. 5,639,949. Alternatively, DNA fragments containing the transgene and the necessary regulatory elements for expression of the transgene can be excised from a plasmid and delivered to the plant cell using microprojectile bombardment-mediated methods.

Zinc-finger nucleases (ZFNs) are also useful in that they allow double strand DNA cleavage at specific sites in plant chromosomes such that targeted gene insertion or deletion can be performed (Shukla et al., 2009, Nature 459: 437-441; Townsend et al., 2009, Nature 459: 442-445).

The CRISPR/Cas9 system (Sander, J. D. and Joung, J. K., Nature Biotechnology, published online Mar. 2, 2014; doi; 10.1038/nbt.2842) is particularly useful for editing plant genomes to modulate the expression of homologous genes encoding enzymes. All that is required to achieve a CRISPR/Cas edit is a Cas enzyme, or other CRISPR nuclease (Murugan et al. (2017), Mol Cell, 68:15), and a single guide RNA (sgRNA) as reviewed extensively by others (Belhag et al. (2015), Curr. Opin. Biotech., 32: 76; Khandagale & Nadaf (2016), Plant Biotechnol Rep, 10:327-343). Several examples of the use of this technology to edit the genomes of plants have now been reported (Belhaj et al. (2013), Plant Methods, 9:39; Zhang et al. (2016), Journal of Genetics and Genomics, 43: 251).

TALENs (transcriptional activator-like effector nucleases), meganucleases, or zinc finger nucleases (ZFNs) can also be used for plant genome editing (Malzahn et al., Cell Biosci, 2017, 7:21; Khandagal & Nadal, Plant Biotechnol Rep, 2016, 10, 327).

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). Also see Weissinger et al. Ann. Rev. Genet. 22:421-477 (1988); Sanford et al. Particulate Science and Technology 5:27-37 (1987) (onion); Christou et al. Plant Physiol. 87:671-674 (1988) (soybean); McCabe et al. (1988) BioTechnology 6:923-926 (soybean); Finer and McMullen In Vitro Cell Dev. Biol. 27P:175-182 (1991) (soybean); Singh et al. Theor. Appl. Genet. 96:319-324 (1998) (soybean); Dafta et al. (1990) Biotechnology 8:736-740 (rice); Klein et al. Proc. Natl. Acad. Sci. USA 85:4305-4309 (1988) (maize); Klein et al. Biotechnology 6:559-563 (1988) (maize); Tomes, U.S. Pat. No. 5,240,855; Buising et al., U.S. Pat. Nos. 5,322,783 and 5,324,646; Tomes et al. (1995) in Plant Cell, Tissue, and Organ Culture: Fundamental Methods, ed. Gamborg (Springer-Verlag, Berlin) (maize); Klein et al. Plant Physiol. 91:440-444 (1988) (maize); Fromm et al. Biotechnology 8:833-839 (1990) (maize); Hooykaas-Van Slogteren et al. Nature 311:763-764 (1984); Bowen et al., U.S. Pat. No. 5,736,369 (cereals); Bytebier et al. Proc. Natl. Acad. Sci. USA 84:5345-5349 (1987) (Liliaceae); De Wet et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler et al. Theor. Appl. Genet. 84:560-566 (1992) (whisker-mediated transformation); D'Halluin et al. Plant Cell 4:1495-1505 (1992) (electroporation); Li et al. Plant Cell Reports 12:250-255 (1993) and Christou and Ford Annals of Botany 75:407-413 (1995) (rice); Osjoda et al. Nature Biotechnology 14:745-750 (1996) (maize via Agrobacterium tumefaciens). References for protoplast transformation and/or gene gun for Agrisoma technology are described in WO 2010/037209. Methods for transforming plant protoplasts are available including transformation using polyethylene glycol (PEG), electroporation, and calcium phosphate precipitation (see for example Potrykus et al., 1985, Mol. Gen. Genet., 199, 183-188; Potrykus et al., 1985, Plant Molecular Biology Reporter, 3, 117-128). Methods for plant regeneration from protoplasts have also been described (Evans et al., in Handbook of Plant Cell Culture, Vol 1, (Macmillan Publishing Co., New York, 1983); Vasil, I K in Cell Culture and Somatic Cell Genetics (Academic, Orlando, 1984)).

Recombinase technologies which are useful for producing the disclosed genetically engineered plants include the cre-lox, FLP/FRT and Gin systems. Methods by which these technologies can be used for the purpose described herein are described for example in (U.S. Pat. No. 5,527,695; Dale and Ow, 1991, Proc. Natl. Acad. Sci. USA 88: 10558-10562; Medberry et al., 1995, Nucleic Acids Res. 23: 485-490).

Transformation protocols as well as protocols for introducing nucleotide sequences into plants may vary depending on the type of plant or plant cell, e.g., monocot or dicot, targeted for transformation.

The transformed cells are grown into plants in accordance with conventional techniques. See, for example, McCormick et al., 1986, Plant Cell Rep. 5: 81-84. These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Procedures for in planta transformation can be simple. Tissue culture manipulations and possible somaclonal variations are avoided and only a short time is required to obtain genetically engineered plants. However, the frequency of transformants in the progeny of such inoculated plants is relatively low and variable. At present, there are very few species that can be routinely transformed in the absence of a tissue culture-based regeneration system. Stable Arabidopsis transformants can be obtained by several in planta methods including vacuum infiltration (Clough & Bent, 1998, The Plant J. 16: 735-743), transformation of germinating seeds (Feldmann & Marks, 1987, Mol. Gen. Genet. 208: 1-9), floral dip (Clough and Bent, 1998, Plant J. 16: 735-743), and floral spray (Chung et al., 2000, Genetically engineered Res. 9: 471-476). Other plants that have successfully been transformed by in planta methods include rapeseed and radish (vacuum infiltration, Ian and Hong, 2001, Genetically engineered Res., 10: 363-371; Desfeux et al., 2000, Plant Physiol. 123: 895-904), Medicago truncatula (vacuum infiltration, Trieu et al., 2000, Plant J. 22: 531-541), camelina (floral dip, WO/2009/117555 to Nguyen et al.), and wheat (floral dip, Zale et al., 2009, Plant Cell Rep. 28: 903-913). In planta methods have also been used for transformation of germ cells in maize (pollen, Wang et al. 2001, Acta Botanica Sin., 43, 275-279; Zhang et al., 2005, Euphytica, 144, 11-22; pistils, Chumakov et al. 2006, Russian J. Genetics, 42, 893-897; Mamontova et al. 2010, Russian J. Genetics, 46, 501-504) and Sorghum (pollen, Wang et al. 2007, Biotechnol. Appl. Biochem., 48, 79-83).

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

The cells that have been transformed may be grown into plants in accordance with conventional techniques. See, for example, McCormick et al. Plant Cell Reports 5:81-84(1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Genetically engineered plants can be produced using conventional techniques to express any genes of interest in plants or plant cells (Methods in Molecular Biology, 2005, vol. 286, Genetically engineered Plants: Methods and Protocols, Pena L., ed., Humana Press, Inc. Totowa, N.J.; Shyamkumar Barampuram and Zhanyuan J. Zhang, Recent Advances in Plant Transformation, in James A. Birchler (ed.), Plant Chromosome Engineering: Methods and Protocols, Methods in Molecular Biology, vol. 701, Springer Science+Business Media). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding a gene of interest is preferably stably transformed and integrated into the genome of the host cells. Transformed cells are preferably regenerated into whole fertile plants. Detailed description of transformation techniques are within the knowledge of those skilled in the art.

In some embodiments, the heterologous polynucleotides of the invention can be transformed into the nucleus using standard techniques known in the art of plant transformation.

Thus, in some embodiments, a heterologous polynucleotide encoding a MQO polypeptide can be transformed into and expressed in the nucleus and the polypeptides produced remain in the cytosol. In other embodiments, a heterologous polynucleotide encoding MQO polynucleotide can be transformed into and expressed in the nucleus, wherein the polypeptides can be targeted to the mitochondria. Thus, in particular embodiments, a heterologous polynucleotide encoding a MQO polypeptide can be operably linked to at least one targeting nucleotide sequence encoding a signal peptide that targets the polypeptides to the mitochondria. Plant mitochondrial targeting sequences for targeting polypeptides into the mitochondria are known in the art. A signal sequence may be operably linked at the N- or C-terminus of a heterologous nucleotide sequence or nucleic acid molecule. Signal peptides (and the targeting nucleotide sequences encoding them) are well known in the art and can be found in public databases such as the “Signal Peptide Website: An Information Platform for Signal Sequences and Signal Peptides.” (website: signalpeptide.de); the “Signal Peptide Database” (website: proline.bic.nus.edu.sg/spdb/index.html) (Choo et al., BMC Bioinformatics 6:249 (2005)(available on website: biomedcentral.com/1471-2105/6/249/abstract); MITOPROT (ihg2.helmholtz-muenchen.de/ihg/mitoprot.html; predicts mitochondrial targeting sequences); PlasMit (gecco.org.chemie.uni-frankfurt.de/plasmit/index.html; predicts mitochondrial transit peptides in Plasmodium falciparum); Predotar (urgi.versailles.inra.fr/predotar/predotar.html; predicts mitochondrial and plastid targeting sequences); SignalP (website: cbs.dtu.dk/services/SignalP/; predicts the presence and location of signal peptide cleavage sites in amino acid sequences from different organisms: Gram-positive prokaryotes, Gram-negative prokaryotes, and eukaryotes). The SignalP method incorporates a prediction of cleavage sites and a signal peptide/non-signal peptide prediction based on a combination of several artificial neural networks and hidden Markov models; and TargetP (website: cbs.dtu.dk/services/TargetP/) predicts the subcellular location of eukaryotic proteins, the location assignment being based on the predicted presence of any of the N-terminal presequences: chloroplast transit peptide (cTP), mitochondrial targeting peptide (mTP) or secretory pathway signal peptide (SP)). (See also, von Heijne, G., Eur J Biochem 133 (1) 17-21 (1983); Martoglio et al. Trends Cell Biol 8 (10):410-5 (1998); Hegde et al. Trends Biochem Sci 31(10):563-71 (2006); Dultz et al. J Biol Chem 283(15):9966-76 (2008); Emanuelsson et al. Nature Protocols 2(4) 953-971(2007); Zuegge et al. 280(1-2):19-26 (2001); Neuberger et al. J Mol Biol. 328(3):567-79 (2003); and Neuberger et al. J Mol Biol. 328(3):581-92 (2003)).

Specific examples of using N-terminal mitochondrial targeting sequences to target microbial or plant proteins to plant mitochondria are disclosed for example by R. S. Allen, K. Tilbrook, A. C. Warden, P. C. Campbell, V. Rolland, S. P. Singh, and C. C. Wood, Expression of 16 nitrogenase proteins within the plant mitochondrial matrix, Front. Plant Sci. 8:287-300 (2017); S. Lee, D. W. Lee, Y. J. Yoo, O. Duncan, Y. J. Oh, Y. J. Lee, G. Lee, J. Whelan, and I. Hwang, Mitochondrial targeting of the Arabidopsis F1-ATPase γ-subunit via multiple compensatory and synergistic presequence motifs, Plant Cell 24:5037-5057 (2012)).

Exemplary mitochondrial signal peptides include, but are not limited to those provided in TABLE 3.

TABLE 3
Amino acid sequences of representative signal peptides.

| Source | Sequence | Target |
|---|---|---|
| Arabidopsis | MLRTVSCLASRSSSSLFFRFFRQFPRSYMSLTS | mitochondria |
| presequence | STAALRVPSRNLRRISSPSVAGRRLLLRRGLRI | and |
| protease1 | PSAAVRSVNGQFSRLSVRA (SEQ ID NO: 6) | chloroplast |
| (AT3G19170) | | |
| Saccharomyces | MLSLRQSIRFFKPATRTLCSSRYLL (SEQ ID | mitochondria |
| cerevisiae cox4 | NO: 7) | |
| Arabidopsis | MYLTASSSASSSIIRAASSRSSSLFSFRSVLSPS | mitochondria |
| aconitase | VSSTSPSSLLARRSFGTISPAFRRWSHSFHSKP | |
| | SPFRFTSQIRA (SEQ ID NO: 8) | |
| Yeast aconitase | MLSARSAIKRPIVRGLATV (SEQ ID NO: 9) | mitochondria |
| Arabidopsis | MAMAVFRREGRRLLPSIAARPIAAIRSPLSSD | mitochondria |
| thaliana | QEEGLLGVRSISTQVVRNRMKSVKNIQKITKA | |
| 77 amino acid | MKMVAASKLRAVQ | |
| targeting sequence | (SEQ ID NO: 10) | |
| from the gamma | | |
| subunit of | | |
| mitochondrial | | |
| ATP synthase | | |
| (GenBank | | |
| Accession | | |
| At2g33040) | | |

Plant promoters can be selected to control the expression of the transgene in different plant tissues or organelles for all of which methods are known to those skilled in the art (Gasser & Fraley, 1989, Science 244: 1293-1299). In one embodiment, promoters are selected from those of eukaryotic or synthetic origin that are known to yield high levels of expression in plants and algae. In a preferred embodiment, promoters are selected from those that are known to provide high levels of expression in monocots.

Constitutive promoters include, for example, the core promoter of the Rsyn7 promoter and other constitutive promoters disclosed in WO 99/43838 and U.S. Pat. No. 6,072,050, the core CaMV 35S promoter (Odell et al., 1985, Nature 313: 810-812), rice actin (McElroy et al., 1990, Plant Cell 2: 163-171), ubiquitin (Christensen et al., 1989, Plant Mol. Biol. 12: 619-632; Christensen et al., 1992, Plant Mol. Biol. 18: 675-689), pEMU (Last et al., 1991, Theor. Appl. Genet. 81: 581-588), MAS (Velten et al., 1984, EMBO J. 3: 2723-2730), and ALS promoter (U.S. Pat. No. 5,659,026). Other constitutive promoters are described in U.S. Pat. Nos. 5,608,149; 5,608,144; 5,604,121; 5,569,597; 5,466,785; 5,399,680; 5,268,463; and 5,608,142.

“Tissue-preferred” promoters can be used to target gene expression within a particular tissue. Tissue-preferred promoters include those described by Van Ex et al., 2009, Plant Cell Rep. 28: 1509-1520; Yamamoto et al., 1997, Plant 12: 255-265; Kawamata et al., 1997, Plant Cell Physiol. 38: 792-803; Hansen et al., 1997, Mol. Gen. Genet. 254: 337-343; Russell et al., 1997, Transgenic Res. 6: 157-168; Rinehart et al., 1996, Plant Physiol. 112: 1331-1341; Van Camp et al., 1996, Plant Physiol. 112: 525-535; Canevascini et al., 1996, Plant Physiol. 112: 513-524; Yamamoto et al., 1994, Plant Cell Physiol. 35: 773-778; Lam, 1994, Results Probl. Cell Differ. 20: 181-196; Orozco et al., 1993, Plant Mol. Biol. 23: 1129-1138; Matsuoka et al., 1993, Proc. Natl. Acad. Sci. USA 90: 9586-9590; and Guevara-Garcia et al., 1993, Plant J. 4: 495-505. Such promoters can be modified, if necessary, for weak expression.

Seed-specific promoters can be used to target gene expression to seeds in particular. Seed-specific promoters include promoters that are expressed in various tissues within seeds and at various stages of development of seeds. Seed-specific promoters can be absolutely specific to seeds, such that the promoters are only expressed in seeds, or can be expressed preferentially in seeds, e.g. at rates that are higher by 2-fold, 5-fold, 10-fold, or more, in seeds relative to one or more other tissues of a plant, e.g. stems, leaves, and/or roots, among other tissues. Seed-specific promoters include, for example, seed-specific promoters of dicots and seed-specific promoters of monocots, among others. For dicots, seed-specific promoters include, but are not limited to, bean β-phaseolin, napin, β-conglycinin, soybean oleosin 1, Arabidopsis thaliana sucrose synthase, flax conlinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, and globulin 1.

Exemplary promoters useful for expression of MQO proteins for specific dicot crops are disclosed in TABLE 4. Examples of promoters useful for increasing the expression of MQO proteins in specific monocot plants are disclosed in TABLE 5. For example, one or more of the promoters from soybean (Glycine max) listed in TABLE 4 may be used to drive the expression of one or more MQO genes encoding the proteins listed in TABLE 1, or the gene sequences in TABLE 2. It may also be useful to increase or otherwise alter the expression of one or more mitochondrial transporters in a specific crop using genome editing approaches as described in Example 7.

TABLE 4
Promoters useful for expression of genes in dicots.

| | | Native organism | Gene ID* |
|---|---|---|---|
| Gene/Promoter | Expression | of promoter | (SEQ ID NO) |
| CaMV 35S | Constitutive | Cauliflower mosaic | (SEQ ID NO: 11) |
| | | virus | |
| Hsp70 | Constitutive | Glycine max | Glyma.02G093200 |
| | | | (SEQ ID NO: 12) |
| Chlorophyll A/B Binding | Constitutive | Glycine max | Glyma.08G082900 |
| Protein (Cab5) | | | (SEQ ID NO: 13) |
| Pyruvate phosphate dikinase | Constitutive | Glycine max | Glyma.06G252400 |
| (PPDK) | | | (SEQ ID NO: 14) |
| Actin | Constitutive | Glycine max | Glyma.19G147900 |
| | | | (SEQ ID NO: 15) |
| ADP-glucose pyrophos- | Seed-specific | Glycine max | Glyma.04G011900 |
| phorylase (AGPase) | | | (SEQ ID NO: 16) |
| Glutelin C (GluC) | Seed-specific | Glycine max | Glyma.03G163500 |
| | | | (SEQ ID NO: 17) |
| β-fructofuranosidase insoluble | Seed-specific | Glycine max | Glyma.17G227800 |
| isoenzyme 1 (CIN1) | | | (SEQ ID NO: 18) |
| MADS-Box | Cob-specific | Glycine max | Glyma.04G257100 |
| | | | (SEQ ID NO: 19) |
| Glycinin (subunit G1) | Seed-specific | Glycine max | Glyma.03G163500 |
| | | | (SEQ ID NO: 20) |
| oleosin isoform A | Seed-specific | Glycine max | Glyma.16G071800 |
| | | | (SEQ ID NO: 21) |
| Hsp70 | Constitutive | Brassica napus | BnaA09g05860D |
| Chlorophyll A/B Binding | Constitutive | Brassica napus | BnaA04g20150D |
| Protein (Cab5) | | | |
| Pyruvate phosphate dikinase | Constitutive | Brassica napus | BnaA01g18440D |
| (PPDK) | | | |
| Actin | Constitutive | Brassica napus | BnaA03g34950D |
| ADP-glucose pyrophos- | Seed-specific | Brassica napus | BnaA06g40730D |
| phorylase (AGPase) | | | |
| Glutelin C (GluC) | Seed-specific | Brassica napus | BnaA09g50780D |
| β-fructofuranosidase insoluble | Seed-specific | Brassica napus | BnaA04g05320D |
| isoenzyme 1 (CIN1) | | | |
| MADS-Box | Cob-specific | Brassica napus | BnaA05g02990D |
| Glycinin (subunit G1) | Seed-specific | Brassica napus | BnaA01g08350D |
| oleosin isoform A | Seed-specific | Brassica napus | BnaC06g12930D |
| 1.7S napin (napA) | Seed-specific | Brassica napus | BnaA01g17200D |

*Gene ID includes sequence information for coding regions as well as associated promoters, 5′ UTRs, and 3′ UTRs and are available at Phytozome (see JGI website phytozome.jgi.doe.gov/pz/portal.html).

TABLE 5
Promoters useful for expression of genes in monocots, including maize and rice.

| Gene/Promoter | Expression | Rice* | Maize* | Other |
|---|---|---|---|---|
| Hsp70 | Constitutive | LOC_Os05g38530* | GRMZM2G310431* | |
| | | (SEQ ID NO: 22) | (SEQ ID NO: 30) | |
| Chlorophyll A/B | Constitutive | LOC_Os01g41710* | AC207722.2_FG009* | |
| Binding Protein | | (SEQ ID NO: 23) | (SEQ ID NO: 31) | |
| (Cab5) | | | GRMZM2G351977 | |
| | | | (SEQ ID NO: 32) | |
| maize ubiquitin | Constitutive | | (SEQ ID NO: 33) | |
| promoter/maize | | | | |
| ubiquitin intron | | | | |
| (sequence listed in | | | | |
| Genbank | | | | |
| KT962835) | | | | |
| maize ubiquitin | Constitutive | | (SEQ ID NO: 34) | |
| promoter/maize | | | | |
| ubiquitin intron | | | | |
| (maize promoter | | | | |
| and intron | | | | |
| sequence with | | | | |
| 99% identity to | | | | |
| sequence in | | | | |
| Genbank | | | | |
| KT985051.1) | | | | |
| CaMV 35S | Constitutive | | | Cauliflower |
| | | | | mosaic virus |
| | | | | (SEQ ID |
| | | | | NO: 11) |
| Pyruvate | Constitutive | LOC_Os05g33570* | GRMZM2G306345* | |
| phosphate | | (SEQ ID NO: 24) | (SEQ ID NO: 35) | |
| dikinase (PPDK) | | | | |
| Actin | Constitutive | LOC_Os03g50885* | GRMZM2G047055* | |
| | | (SEQ ID NO: 25) | (SEQ ID NO: 36) | |
| Hybrid | Constitutive | N/A | SEQ ID NO: 37 | |
| cab5/hsp70 intron | | | | |
| promoter | | | | |
| ADP-glucose | Seed-specific | LOC_Os01g44220* | GRMZM2G429899* | |
| pyrophos- | | (SEQ ID NO: 26) | (SEQ ID NO: 38) | |
| phorylase | | | | |
| (AGPase) | | | | |
| Glutelin C (GluC) | Seed-specific | LOC_Os02g25640* | N/A | |
| | | (SEQ ID NO: 27) | | |
| β- | Seed-specific | LOC_Os02g33110* | GRMZM2G139300* | |
| fructofuranosidase | | (SEQ ID NO: 28) | (SEQ ID NO: 39) | |
| insoluble | | | | |
| isoenzyme 1 | | | | |
| (CIN1) | | | | |
| MADS-Box | Cob-specific | LOC_Os12g10540* | GRMZM2G160687* | |
| | | (SEQ ID NO: 29) | (SEQ ID NO: 40) | |
| Maize TrpA | Seed-specific | | GRMZM5G841619 | |
| promoter | | | (SEQ ID NO: 41) | |

*Gene ID includes sequence information for coding regions as well as associated promoters, 5′ UTRs, and 3′ UTRs and are available at Phytozome (see JGI website phytozome.jgi.doe.gov/pz/portal.html).

Certain embodiments use genetically engineered plants or plant cells having multi-gene expression constructs harboring more than one transgene and promoter. The promoters can be the same or different.

Any of the described promoters can be used to control the expression of one or more of genes, their homologs and/or orthologs as well as any other genes of interest in a defined spatiotemporal manner.

Nucleic acid sequences intended for expression in genetically engineered plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra. The following is a description of various components of typical expression cassettes.

A variety of transcriptional terminators are available for use in expression cassettes. These are responsible for the termination of transcription beyond the transgene and the correct polyadenylation of the transcripts. Appropriate transcriptional terminators are those that are known to function in plants and include the CaMV 35S terminator, the tml terminator, the nopaline synthase terminator and the pea rbcS E9 terminator. These are used in both monocotyledonous and dicotyledonous plants.

The coding sequence of the selected gene may be genetically engineered by altering the coding sequence for optimal expression in the crop species of interest. Methods for modifying coding sequences to achieve optimal expression in a particular crop species are well known (Perlak et al., 1991, Proc. Natl. Acad. Sci. USA 88: 3324 and Koziel et al., 1993, Biotechnology 11: 194-200).

Individual plants within a population of genetically engineered plants that express a recombinant gene(s) may have different levels of gene expression. The variable gene expression is due to multiple factors including multiple copies of the recombinant gene, chromatin effects, and gene suppression. Accordingly, a phenotype of the genetically engineered plant may be measured as a percentage of individual plants within a population. The yield of a plant can be measured simply by weighing. The yield of seed from a plant can also be determined by weighing. The increase in seed weight from a plant can be due to a number of factors, including an increase in the number or size of the seed pods, an increase in the number of seed and/or an increase in the number of seed per plant. In the laboratory or greenhouse seed yield is usually reported as the weight of seed produced per plant and in a commercial crop production setting yield is usually expressed as weight per acre or weight per hectare.

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A genetically engineered plant can be produced by selection of transformed seeds or by selection of transformed plant cells and subsequent regeneration.

In some embodiments, the genetically engineered plants are grown (e.g., on soil) and harvested. In some embodiments, above ground tissue is harvested separately from below ground tissue. Suitable above ground tissues include shoots, stems, leaves, flowers, grain, and seed. Exemplary below ground tissues include roots and root hairs. In some embodiments, whole plants are harvested and the above ground tissue is subsequently separated from the below ground tissue.

Genetic constructs may encode a selectable marker to enable selection of transformation events. There are many methods that have been described for the selection of transformed plants (for review see Miki et al., Journal of Biotechnology, 2004, 107, 193-232, and references incorporated therein). Selectable marker genes that have been used extensively in plants include the neomycin phosphotransferase gene nptll (U.S. Pat. Nos. 5,034,322, 5,530,196), hygromycin resistance gene (U.S. Pat. No. 5,668,298, Waldron et al., (1985), Plant Mol Biol, 5:103-108; Zhijian et al., (1995), Plant Sci, 108:219-227), the bar gene encoding resistance to phosphinothricin (U.S. Pat. No. 5,276,268), the expression of aminoglycoside 3′-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Other suitable selectable markers include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); streptomycin (Jones et al., (1987), Mol Gen Genet, 210:86-91); bleomycin (Hille et al., (1990), Plant Mol Biol, 7:171-176); sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J, 6:2513-2518).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-458). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of genetically engineered plants.

Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein.

Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296). Improved versions of many of the fluorescent proteins have been made for various applications. It will be apparent to those skilled in the art how to use the improved versions of these proteins, including combinations, for selection of transformants.

The plants modified for enhanced yield may have stacked input traits that include herbicide resistance and insect tolerance, for example a plant that is tolerant to the herbicide glyphosate and that produces the Bacillus thuringiensis (BT) toxin. Glyphosate is a herbicide that prevents the production of aromatic amino acids in plants by inhibiting the enzyme 5-enolpyruvylshikimate-3-phosphate synthase (EPSP synthase). The overexpression of EPSP synthase in a crop of interest allows the application of glyphosate as a weed killer without killing the modified plant (Suh, et al., J. M Plant Mol. Biol. 1993, 22, 195-205). BT toxin is a protein that is lethal to many insects providing the plant that produces it protection against pests (Barton, et al. Plant Physiol. 1987, 85, 1103-1109). Other useful herbicide tolerance traits include but are not limited to tolerance to Dicamba by expression of the dicamba monoxygenase gene (Behrens et al, 2007, Science, 316, 1185), tolerance to 2,4-D and 2,4-D choline by expression of a bacterial aad-1 gene that encodes for an aryloxyalkanoate dioxygenase enzyme (Wright et al., Proceedings of the National Academy of Sciences, 2010, 107, 20240), glufosinate tolerance by expression of the bialophos resistance gene (bar) or the pat gene encoding the enzyme phosphinotricin acetyl transferase (Droge et al., Planta, 1992, 187, 142), as well as genes encoding a modified 4-hydroxyphenylpyruvate dioxygenase (HPPD) that provides tolerance to the herbicides mesotrione, isoxaflutole, and tembotrione (Siehl et al., Plant Physiol, 2014, 166, 1162).

### EXAMPLES

### Example 1. MQO, a Bacterial Enzyme with Favorable Thermodynamics for Conversion of Malate to Oxaloacetate

In the seed, the tricarboxylic acid (TCA) cycle is expected to operate in mitochondria to provide NADH and ATP from sugar metabolism (FIG. 1). In this case, malate must be converted to oxaloacetate by malate dehydrogenase (MDH). Plants typically contain only NAD(P)H-dependent MDHs, and these enzymes catalyze reactions with thermodynamics that greatly favor malate formation, even when the [NAD⁺]/[NADH] ratio is high (FIG. 1A). MDHs with NAD(P)H as cofactor are soluble enzymes and thus are not linked directly to respiration as is succinate dehydrogenase, which can proceed in an unfavorable thermodynamic direction with ease because it is coupled to a reaction (donation of electrons to oxygen) that is extremely favorable, such that the overall thermodynamics actually favor electron donation.

Experimental metabolic flux analysis data (Iyer et al., Plant, Cell and Environment 31:506-517, 2008; Allen et al., Plant J. 58:220-234, 2009; Sriram et al., Plant Physiol. 136:3043-3057, 2004; Alonso et al. Plant J. 52:296-308, 2007) show that despite its unfavorability, often a flux from malate to oxaloacetate in seed mitochondria still apparently occurs (FIG. 1B), most likely by extensive physical association of proteins to link favorable reactions to unfavorable ones (see, e.g., Zhang et al., Plant Physiol. 177:966-979, 2018). These data do not typically show the action of a di- or tricarboxylate transporter interrupting this flux, suggesting that these transporters are often not significantly active in seed tissue. This points to MDH as a possible rate limiter in plant mitochondria.

For relief of rate limitation at the malate dehydrogenase step of the TCA cycle in mitochondria, the expression of a malate dehydrogenase that is associated with a better electron acceptor than NAD(P)⁺, such as a quinone-utilizing variety can be used. Bacteria routinely use malate:quinone oxidoreductase (Mqo; EC 1.1.5.4, FIG. 1B) for malate oxidation to complete the TCA cycle even though the resulting electrons are ultimately lost to oxygen rather than being harnessed for biosynthesis. Bacteria, unlike plants, are subject to fierce competition for carbon sources, and thus it is often more advantageous for them to utilize carbon quickly than to use carbon particularly efficiently.

Several studies suggest that the yield of sink tissue such as seed is limited under some circumstances by the strength of the sink demand. In this sense, kinetic improvement of the TCA cycle via Mqo, even though it would not necessarily increase carbon efficiency, could induce higher overall photosynthate production at source tissues such as leaves. This would lead to an overall improvement in sink-tissue production.

Numerous bacterial examples of Mqo are known and the examples mentioned in the text for which sequences are known are listed in TABLE 1, though this is meant to be illustrative of the many possible sources and by no means an exhaustive list. A BLAST search using the Corynebacterium glutamicum Mqo protein sequence was performed to find Mqo homologs in higher plants (TABLE 2). Both blastp and tblastn searches at the NCBI BLAST website (website: blast.ncbi.nlm.nih.gov/Blast.cgi) were performed for green plants using the nr, TSA, and wgs databases. It should be noted that the Corynebacterium glutamicum MDH protein (SEQ ID NO: 1) is quite dissimilar from its Mqo protein (SEQ ID NO: 2) (FIG. 2) and therefore BLAST hits to Mqo are not likely to be MDH proteins. The best hit from each distinct plant species is listed in TABLE 2; this listing is once again illustrative but by no means exhaustive. The MQO protein from Corynebacterium glutamicum (SEQ ID NO: 2) was chosen for expression in plants.

### Example 2. Design of Constructs for Expression of MQO in Plants

To target the MQO protein from Corynebacterium glutamicum (SEQ ID NO: 2) to the mitochondria, a gene cassette was designed containing an N-terminal mitochondrial targeting signal fused to the mqo gene. For the targeting sequence, a genetic fragment (SEQ ID NO: 42) encoding the 77 amino acid N-terminal mitochondrial targeting sequence from the Arabidopsis thaliana gamma subunit of the mitochondrial ATP synthase (SEQ ID NO: 10) was used. For the mqo sequence, the ATG start site of the gene encoding the MQO protein from Corynebacterium glutamicum was removed and the remainder of the gene was codon optimized for expression in plants using codon optimization for Arabidopsis thaliana. The DNA sequence and amino acid sequence of the final fusion are shown in SEQ ID NO: 43 and SEQ ID: NO 44, respectively.

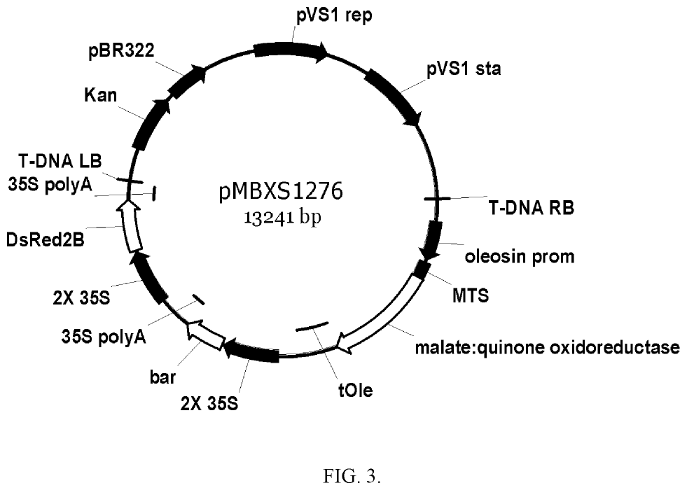

For transformation of canola (Brassica napus) and Camelina sativa, genetic construct pMBXS1276 (FIG. 3; SEQ ID NO: 51) was designed containing a seed-specific expression cassette, driven by the promoter from the soya bean oleosin isoform A gene, for expression of the mitochondrial targeted MQO (mt-MQO) protein in plants. The MQO sequence was codon optimized based on preferred codon usage for Arabidopsis thaliana.

### Example 3. Seed Specific Expression of Mt-MQO in Camelina sativa

Construct pMBXS1276 was transformed into Camelina sativa cv CS0043 (abbreviated as WT43) using a floral dip procedure as follows.

In preparation for plant transformation experiments, seeds of Camelina sativa germplasm 10CS0043 (abbreviated WT43, obtained from Agriculture and Agri-Food Canada) were sown directly into 4 inch (10 cm) pots filled with soil in the greenhouse. Growth conditions were maintained at 24° C. during the day and 18° C. during the night. Plants were grown until flowering. Plants with a number of unopened flower buds were used in ‘floral dip’ transformations.

Agrobacterium strain GV3101 (pMP90) was transformed with genetic construct pMBXS1276 using electroporation. A single colony of GV3101 (pMP90) containing pMBXS1276 was obtained from a freshly streaked plate and was inoculated into 5 mL LB medium. After overnight growth at 28° C., 2 mL of culture was transferred to a 500-mL flask containing 300 mL of LB and incubated overnight at 28° C. Cells were pelleted by centrifugation (6,000 rpm, 20 min), and diluted to an OD600 of ˜0.8 with infiltration medium containing 5% sucrose and 0.05% (v/v) Silwet-L77 (Lehle Seeds, Round Rock, Tex., USA). Camelina plants were transformed by “floral dip” using the transformation construct as follows. Pots containing plants at the flowering stage were placed inside a 460 mm height vacuum desiccator (Bel-Art, Pequannock, N.J., USA). Inflorescences were immersed into the Agrobacterium inoculum contained in a 500-ml beaker. A vacuum (85 kPa) was applied and held for 5 min. Plants were removed from the desiccator and were covered with plastic bags in the dark for 24 h at room temperature. Plants were removed from the bags and returned to normal growth conditions within the greenhouse for seed formation (T1 generation of seed).

T1 seeds were obtained and screened for the expression of the visual marker DsRed, a marker on the T-DNA in plasmid vector pMBXS1276 (FIG. 3). 37 independent transgenic events were identified. The Dsred positive T1 lines were grown in the greenhouse along with the wild type controls. Agronomic and yield evaluation of multiple plants is performed in the T2 generation on single copy and multiple copy lines. T3 seed is collected and seed yield and oil content is determined. The oil content of T3 seeds is measured using published procedures for preparation of fatty acid methyl esters (Malik et al. 2015, Plant Biotechnology Journal, 13, 675-688).

### Example 4. Seed Specific Expression of Mt-MQO in Canola

Canola is transformed with construct pMBXS1276 expressing the MQO protein as follows.

In preparation for plant transformation experiments, seeds of Brassica napus cv DH12075 (obtained from Agriculture and Agri-Food Canada) were surface sterilized with sufficient 95% ethanol for 15 seconds, followed by 15 minutes incubation with occasional agitation in full strength Javex (or other commercial bleach, 7.4% sodium hypochlorite) and a drop of wetting agent such as Tween 20. The Javex solution was decanted and 0.025% mercuric chloride with a drop of Tween 20 was added and the seeds were sterilized for another 10 minutes. The seeds were then rinsed three times with sterile distilled water. The sterilized seeds were plated on half strength hormone-free Murashige and Skoog (MS) media (Murashige T, Skoog F (1962). Physiol Plant 15:473-498) with 1% sucrose in 15×60 mm petri dishes that were then placed, with the lid removed, into a larger sterile vessel (Majenta GA7 jars). The cultures were kept at 25° C., with 16 h light/8 h dark, under approx. 70-80 μE of light intensity in a tissue culture cabinet. 4-5 days old seedlings were used to excise fully unfolded cotyledons along with a small segment of the petiole. Excisions were made so as to ensure that no part of the apical meristem was included.

Agrobacterium strain GV3101 (pMP90) carrying construct pMBXS1276 was grown overnight in 5 ml of LB media with 50 mg/L kanamycin, gentamycin, and rifampicin. The culture was centrifuged at 2000 g for 10 min., the supernatant was discarded and the pellet was suspended in 5 ml of inoculation medium (Murashige and Skoog with B5 vitamins [MS/B5; Gamborg O L, Miller R A, Ojima K. Exp Cell Res 50:151-158], 3% sucrose, 0.5 mg/L benzyl aminopurine (BA), pH 5.8). Cotyledons were collected in Petri dishes with ˜1 ml of sterile water to keep them from wilting. The water was removed prior to inoculation and explants were inoculated in a mixture of 1 part Agrobacterium suspension and 9 parts inoculation medium in a final volume sufficient to bathe the explants. After explants were well exposed to the Agrobacterium solution and inoculated, a pipet was used to remove any extra liquid from the petri dishes.

The Petri plates containing the explants incubated in the inoculation media were sealed and kept in the dark in a tissue culture cabinet set at 25° C. After 2 days the cultures were transferred to 4° C. and incubated in the dark for 3 days. The cotyledons, in batches of 10, were then transferred to selection medium consisting of Murashige Minimal Organics (Sigma), 3% sucrose, 4.5 mg/L BA, 500 mg/L MES, 27.8 mg/L Iron (II) sulfate heptahydrate, pH 5.8, 0.7% Phytagel with 300 mg/L timentin, and 2 mg/L L-phosphinothricin (L-PPT) added after autoclaving. The cultures were kept in a tissue culture cabinet set at 25° C., 16 h/8 h, with a light intensity of about 125 μmol m⁻² s⁻¹. The cotyledons were transferred to fresh selection every 3 weeks until shoots were obtained. The shoots were excised and transferred to shoot elongation media containing MS/B5 media, 2% sucrose, 0.5 mg/L BA, 0.03 mg/L gibberellic acid (GA₃), 500 mg/L 4-morpholineethanesulfonic acid (MES), 150 mg/L phloroglucinol, pH 5.8, 0.9% Phytagar and 300 mg/L timentin and 3 mg/L L-phosphinothricin added after autoclaving. After 3-4 weeks any callus that formed at the base of shoots with normal morphology was cut off and shoots were transferred to rooting media containing half strength MS/B5 media with 1% sucrose and 0.5 mg/L indole butyric acid, 500 mg/L MES, pH 5.8, 0.8% agar, with 1.5 mg/L L-PPT and 300 mg/L timentin added after autoclaving. The plantlets with healthy shoots were hardened and transferred to 6 inch (15 cm) pots in the greenhouse. 148 T0 lines transformed with pMBXS1276 were generated and are being grown in the greenhouse. 24 single copy lines were identified. Plants are allowed to grow in the greenhouse produce T1 transgenic seeds, which are then collected.

Screening of transgenic plants of canola expressing the MQO protein from pMBXS1276 to identify plants with higher yield is performed as follows. The T1 seeds of several independent lines are grown in a randomized complete block design in a greenhouse maintained at 24° C. during the day and 18° C. during the night. The T2 generation of seed from each line is harvested. Seed yield from each plant is determined by harvesting all of the mature seeds from a plant and drying them in an oven with mechanical convection set at 22° C. for two days. The weight of the entire harvested seed is recorded. The 100 seed weight is measured to obtain an indication of seed size. The oil content of seeds is measured using published procedures for preparation of fatty acid methyl esters (Malik et al. 2015, Plant Biotechnology Journal, 13, 675-688).

### Example 5. Seed Specific Expression of Mt-MQO in Maize

An expression cassette for the mt-mqo gene can be constructed using a variety of different promoters for expression in maize. Candidate constitutive and seed-specific promoters for use in monocots including corn are listed in TABLE 5, however those skilled in the art will understand that other promoters can be selected for expression.

In some instances, it may be advantageous to create a hybrid promoter containing a promoter sequence and an intron. These promoters can deliver higher levels of stable expression. Examples of such hybrid promoters include the hybrid maize Cab-m5 promoter/maize hsp70 intron (SEQ ID NO: 37, TABLE 5) and the maize ubiquitin promoter/maize ubiquitin intron (SEQ ID NO: 33 and 34, TABLE 5).

An example expression cassette for seed specific expression of the mt-mqo gene in maize includes the genetic elements in TABLE 6 (Expression Cassette 1), in which the promoter is operably linked to the mt-mqo gene which is operably linked to the termination sequence. Expression cassette 2 (TABLE 6) contains a bar gene driven by the maize ubiquitin promoter/maize ubiquitin intron, conferring glufosinate tolerance or bialophos resistance for selection of transformants. These expression cassettes can be transformed into maize protoplasts, calli, or immature embryos using biolistics as reviewed in Que et al., 2014, either by delivery on a single DNA fragment or co-transformation of two DNA fragments.

TABLE 6
Transformation cassettes for seed specific expression of the mt-mqo gene in maize

| Expression Cassette 1: | | | Expression Cassette 2: | | |
|---|---|---|---|---|---|
| mt-mqo expression cassette | | | Selectable marker expression cassette | | |
| Promoter | Gene | Terminator | Promoter | Gene | Terminator |
| Maize trpA | Mt-MQO | Maize trpA | Maize ubiquitin | Bar conferring | Maize ubiquitin |
| promoter | gene | 3′ UTR | promoter/maize | bialophos | 3′UTR |
| (SEQ ID NO: 41) | (SEQ ID NO: 43) | (SEQ ID NO: 45) | ubiquitin intron | tolerance | (SEQ ID NO: 47) |
| | | | (SEQ ID NO: 33) | (SEQ ID NO: 46) | |

It will be apparent to those skilled in the art that many selectable markers can be used in maize transformations for the mt-mqo expression cassette described in TABLE 6 that are not derived from plant pest sequences for selection purposes. These include maize acetolactate synthase/acetohydroxy acid synthase (ALS/AHAS) mutant genes conferring resistance to a range of herbicides from the ALS family of herbicides, including chlorsulfuron and imazethapyr; a 5-enolpyruvoylshikimate-3-phosphate synthase (EPSPS) mutant gene from maize, providing resistance to glyphosate; as well as multiple other selectable markers that are all reviewed in Que et al., 2014 (Que, Q. et al., Front. Plant Sci. 5 Aug. 2014; doi.org/10.3389/fpls.2014.00379).

Methods to transform the expression cassette described in TABLE 6 into maize are routine and well known in the art and have recently been reviewed by Que et al., (2014), Frontiers in Plant Science 5, article 379, pp 1-19.

Protoplast transformation methods useful for practicing the invention are well known to those skilled in the art. Such procedures include for example the transformation of maize protoplasts as described by Rhodes and Gray (Rhodes, C. A. and D. W. Gray, Transformation and regeneration of maize protoplasts, in Plant Tissue Culture Manual: Supplement 7, K. Lindsey, Editor. 1997, Springer Netherlands: Dordrecht. p. 353-365). For protoplast transformation of maize, the expression cassettes described in TABLE 6 can be co-bombarded, or delivered on a single DNA fragment. The bar gene imparting transgenic plants resistance to bialophos is used for selection.

For Agrobacterium-mediated transformation of maize, the expression cassettes described in TABLE 6 can be inserted into a binary vector. The binary vector is transformed into an Agrobacterium tumefaciens strain, such as A. tumefaciens strain EHA101. Agrobacterium-mediated transformation of maize can be performed following a previously described procedure (Frame et al. (2006), Agrobacterium Protocols, Wang K., ed., Vol. 1, pp 185-199, Humana Press) as follows.

Plant Material: Plants grown in a greenhouse are used as an explant source. Ears are harvested 9-13 days after pollination and surface sterilized with 80% ethanol.

Explant Isolation, Infection and Co-Cultivation: Immature zygotic embryos (1.2-2.0 mm) are aseptically dissected from individual kernels and incubated in an A. tumefaciens strain EHA101 culture containing the transformation vector (grown in 5 ml N6 medium supplemented with 100 μM acetosyringone for stimulation of the bacterial vir genes for 2-5 h prior to transformation) at room temperature for 5 min. The infected embryos are transferred scutellum side up on to a co-cultivation medium (N6 agar-solidified medium containing 300 mg/l cysteine, 5 μM silver nitrate and 100 μM acetosyringone) and incubated at 20° C., in the dark for 3 d. Embryos are transferred to N6 resting medium containing 100 mg/l cefotaxime, 100 mg/l vancomycin and 5 μM silver nitrate and incubated at 28° C., in the dark for 7 d.

Callus Selection: All embryos are transferred on to the first selection medium (the resting medium described above supplemented with 1.5 mg/l bialaphos) and incubated at 28° C. in the dark for 2 weeks followed by subculture on a selection medium containing 3 mg/l bialaphos. Proliferating pieces of callus are propagated and maintained by subculture on the same medium every 2 weeks.

Plant Regeneration and Selection: Bialaphos-resistant embryogenic callus lines are transferred on to regeneration medium I (MS basal medium supplemented with 60 g/l sucrose, 1.5 mg/l bialaphos and 100 mg/l cefotaxime and solidified with 3 g/l Gelrite) and incubated at 25° C. in the dark for 2 to 3 weeks. Mature embryos formed during this period are transferred on to regeneration medium II (the same as regeneration medium I with 3 mg/l bialaphos) for germination in the light (25° C., 80-100 μmol/m²/s light intensity, 16/8-h photoperiod). Regenerated plants are ready for transfer to soil within 10-14 days. Plants are grown in the greenhouse to maturity and T1 seeds are isolated.

The copy number of the transgene insert is determined, through methods such as Southern blotting or digital PCR, and lines are selected to bring forward for further analysis. Overexpression of the mt-MQO gene is determined by RT-PCR and/or Western blotting techniques and plants with the desired level of expression are selected. Homozygous lines are generated. The yield seed of homozygous lines is compared to control lines.

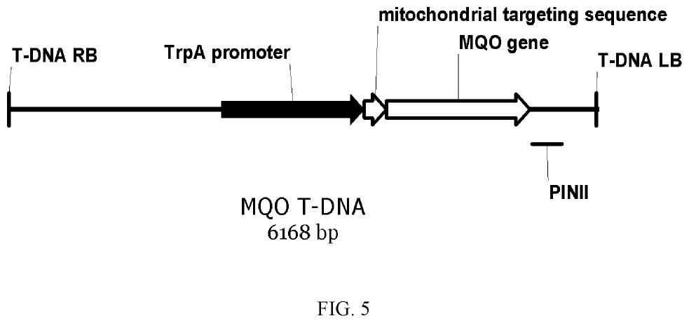

A transformation construct for Agrobacterium mediated transformation of maize with the mqo gene from Corynebacterium glutamicum was prepared to target the MQO protein to the mitochondria of seed. The expression cassette for mqo in the construct contained: a maize trpA promoter (SEQ ID NO: 41); an N-terminal mitochondrial targeting sequence from the Arabidopsis F-ATPase gamma subunit codon optimized for maize; the mqo gene from Corynebacterium glutamicum codon optimized for maize; and the PINII termination sequence. This cassette was inserted into an appropriate binary vector and transformed into the maize inbred line HC69 using a contract service provider. The expected T-DNA insert from this transformation is shown in FIG. 5 (SEQ ID NO: 52). 468 embryos were obtained from this transformation. 191 regenerated T0 plantlets were obtained of which 31 plantlets contained a single copy DNA insertion. T0 lines were transferred to a greenhouse for further growth. Heterozygous T1 seeds are obtained by growing the T0 plants to maturity in the greenhouse and collecting seed. T1 seeds are planted in the greenhouse and homozygous and null lines are identified and are crossed with elite germplasm to make hybrids. Hybrid seed is harvested and is used to plant a field trial (randomized complete block design). Differences in agronomic performance and seed yield are measured.

### Example 6. Seed Specific Expression of Mt-MQO in Soybean

For seed specific expression of the mt-MQO gene in soybean, the expression cassettes described in TABLE 7 are constructed using cloning techniques standard for those skilled in the art. In TABLE 7, the mt-MQO gene codon optimized for Arabidopsis thaliana is used but codon usage can be alternatively optimized for soybean. It will be apparent to those skilled in the art that many different promoters are available for expression in plants. TABLE 4 lists additional options for use in dicots that can be used as alternate promoters for expression cassettes described in TABLE 7.

TABLE 7
Transformation cassettes for seed specific expression of the mt-mqo gene in soybean

| Expression Cassette 1: | | | Expression Cassette 2: | | |
|---|---|---|---|---|---|
| mt-mqo expression cassette | | | Selectable marker expression cassette | | |
| Promoter | Gene | Terminator | Promoter | Gene | Terminator |
| seed-specific | Mt-MQO gene | Terminator from | soybean actin | Hygromycin gene | 3′ UTRfrom |
| promoter from | (SEQ ID NO: 43) | the soya bean | promoter | containing the | the soybean |
| the soya bean | | oleosin isoform | (SEQ ID NO: 15) | cat-1 intron | actin gene |
| oleosin isoform | | A gene | | from the bean | (SEQ ID NO: 50). |
| A gene | | (SEQ ID NO: 48) | | catalase-1 gene | |
| (SEQ ID NO: 21) | | | | (SEQ ID NO: 49) | |

### Soybean Transformation

Transformation can occur via biolistic or Agrobacterium-mediated transformation procedures.

For biolistic transformation, the purified expression cassette for the mt-MQO gene is co-bombarded with the expression cassette for the hygromycin resistance gene into embryogenic cultures of soybean Glycine max cultivars X5 and Westag97, to obtain transgenic plants.

The transformation, selection, and plant regeneration protocol is adapted from Simmonds (2003) (Simmonds, 2003, Genetic Transformation of Soybean with Biolistics. In: Jackson J F, Linskens H F (eds) Genetic Transformation of Plants. Springer Verlag, Berlin, pp 159-174) and is performed as follows.

Induction and Maintenance of Proliferative Embryogenic Cultures: Immature pods, containing 3-5 mm long embryos, are harvested from host plants grown at 28/24° C. (day/night), 15-h photoperiod at a light intensity of 300-400 μmol m⁻² s⁻¹. Pods are sterilized for 30 s in 70% ethanol followed by 15 min in 1% sodium hypochlorite [with 1-2 drops of Tween 20 (Sigma, Oakville, ON, Canada)] and three rinses in sterile water. The embryonic axis is excised and explants are cultured with the abaxial surface in contact with the induction medium [MS salts, B5 vitamins (Gamborg O L, Miller R A, Ojima K. Exp Cell Res 50:151-158), 3% sucrose, 0.5 mg/L BA, pH 5.8), 1.25-3.5% glucose (concentration varies with genotype), 20 mg/l 2,4-D, pH 5.7]. The explants, maintained at 20° C. at a 20-h photoperiod under cool white fluorescent lights at 35-75 μmol m⁻² s⁻¹, are sub-cultured four times at 2-week intervals. Embryogenic clusters, observed after 3-8 weeks of culture depending on the genotype, are transferred to 125-ml Erlenmeyer flasks containing 30 ml of embryo proliferation medium containing 5 mM asparagine, 1-2.4% sucrose (concentration is genotype dependent), 10 mg/l 2,4-D, pH 5.0 and cultured as above at 35-60 μmol m⁻² s⁻¹ of light on a rotary shaker at 125 rpm. Embryogenic tissue (30-60 mg) is selected, using an inverted microscope, for subculture every 4-5 weeks.

Transformation: Cultures are bombarded 3 days after subculture. The embryogenic clusters are blotted on sterile Whatman filter paper to remove the liquid medium, placed inside a 10×30-mm Petri dish on a 2×2 cm² tissue holder (PeCap, 1 005 μm pore size, Band S H Thompson and Co. Ltd. Scarborough, ON, Canada) and covered with a second tissue holder that is then gently pressed down to hold the clusters in place. Immediately before the first bombardment, the tissue is air dried in the laminar air flow hood with the Petri dish cover off for no longer than 5 min. The tissue is turned over, dried as before, bombarded on the second side and returned to the culture flask. The bombardment conditions used for the Biolistic PDS-I000/He Particle Delivery System are as follows: 737 mm Hg chamber vacuum pressure, 13 mm distance between rupture disc (Bio-Rad Laboratories Ltd., Mississauga, ON, Canada) and macrocarrier. The first bombardment uses 900 psi rupture discs and a microcarrier flight distance of 8.2 cm, and the second bombardment uses 1100 psi rupture discs and 11.4 cm microcarrier flight distance. DNA precipitation onto 1.0 μm diameter gold particles is carried out as follows: 2.5 μl of 100 ng/μl of DNA encoding the expression cassette for mt-MQO (TABLE 7; expression construct 1) and 2.5 μl of 100 ng/μl selectable marker DNA (cassette for hygromycin selection, TABLE 7; expression construct 2) are added to 3 mg gold particles suspended in 50 μl sterile dH₂O and vortexed for 10 sec; 50 μl of 2.5 M CaCl₂ is added, vortexed for 5 sec, followed by the addition of 20 μl of 0.1 M spermidine which is also vortexed for 5 sec. The gold is then allowed to settle to the bottom of the microfuge tube (5-10 min) and the supernatant fluid is removed. The gold/DNA is resuspended in 200 μl of 100% ethanol, allowed to settle and the supernatant fluid is removed. The ethanol wash is repeated and the supernatant fluid is removed. The sediment is resuspended in 120 μl of 100% ethanol and aliquots of 8 μl are added to each macrocarrier. The gold is resuspended before each aliquot is removed. The macrocarriers are placed under vacuum to ensure complete evaporation of ethanol (about 5 min).

Selection: The bombarded tissue is cultured on embryo proliferation medium described above for 12 days prior to subculture to selection medium (embryo proliferation medium contains 55 mg/l hygromycin added to autoclaved media). The tissue is sub-cultured 5 days later and weekly for the following 9 weeks. Green colonies (putative transgenic events) are transferred to a well containing 1 ml of selection media in a 24-well multi-well plate that is maintained on a flask shaker as above. The media in multi-well dishes is replaced with fresh media every 2 weeks until the colonies are approx. 2-4 mm in diameter with proliferative embryos, at which time they are transferred to 125 ml Erlenmeyer flasks containing 30 ml of selection medium. A portion of the proembryos from transgenic events is harvested to examine gene expression by RT-PCR.

Plant regeneration: Maturation of embryos is carried out, without selection, at conditions described for embryo induction. Embryogenic clusters are cultured on Petri dishes containing maturation medium (MS salts, B5 vitamins, 6% maltose, 0.2% gelrite gellan gum (Sigma), 750 mg/l MgCl₂, pH 5.7) with 0.5% activated charcoal for 5-7 days and without activated charcoal for the following 3 weeks. Embryos (10-15 per event) with apical meristems are selected under a dissection microscope and cultured on a similar medium containing 0.6% phytagar (Gibco, Burlington, ON, Canada) as the solidifying agent, without the additional MgCl₂, for another 2-3 weeks or until the embryos become pale yellow in color. A portion of the embryos from transgenic events after varying times on gelrite are harvested to examine gene expression by RT-PCR.

Mature embryos are desiccated by transferring embryos from each event to empty Petri dish bottoms that are placed inside Magenta boxes (Sigma) containing several layers of sterile Whatman filter paper flooded with sterile water, for 100% relative humidity. The Magenta boxes are covered and maintained in darkness at 20° C. for 5-7 days. The embryos are germinated on solid B5 medium containing 2% sucrose, 0.2% gelrite and 0.075% MgCl₂ in Petri plates, in a chamber at 20° C., 20-h photoperiod under cool white fluorescent lights at 35-75 μmol m⁻² s⁻¹. Germinated embryos with unifoliate or trifoliate leaves are planted in artificial soil (Sunshine Mix No. 3, SunGro Horticulture Inc., Bellevue, Wash., USA), and covered with a transparent plastic lid to maintain high humidity. The flats are placed in a controlled growth cabinet at 26/24° C. (day/night), 18 h photoperiod at a light intensity of 150 μmol m⁻² s⁻¹. At the 2-3 trifoliate stage (2-3 weeks), the plantlets with strong roots are transplanted to pots containing a 3:1:1:1 mix of ASB Original Grower Mix (a peat-based mix from Greenworld, ON, Canada):soil:sand:perlite and grown at 18-h photoperiod at a light intensity of 300-400 μmol m⁻² s⁻¹.

T1 seeds are harvested and planted in soil and grown in a controlled growth cabinet at 26/24° C. (day/night), 18 h photoperiod at a light intensity of 300-400 μmol m⁻² s⁻¹. Plants are grown to maturity and T2 seed is harvested. Seed yield per plant and oil content of the seeds is measured.

The selectable marker can be removed by segregation if desired by identifying co-transformed plants that have not integrated the selectable marker expression cassette and the mt-MQO gene cassette into the same locus. In this case, plants are grown, allowed to set seed and germinated. Leaf tissue is harvested from soil grown plants and screened for the presence of the selectable marker cassette. Plants containing only the mt-MQO gene expression cassette are advanced.

### Example 7. Use of Genome Editing to Insert Mt-MQO into the Genome of Plants

There are multiple methods to achieve double stranded breaks in genomic DNA, including the use of zinc finger nucleases (ZFN), transcription activator-like effector nucleases (TALENs), engineered meganucleases, and the CRISPR/Cas system (CRISPR is an acronym for clustered, regularly interspaced, short, palindromic repeats and Cas an abbreviation for CRISPR-associated protein) (for review see Khandagal & Nadal, Plant Biotechnol Rep, 2016, 10, 327). CRISPR/Cas mediated genome editing is the easiest of the group to implement since all that is needed is the Cas9 enzyme and a single guide RNA (sgRNA) with homology to the modification target to direct the Cas9 enzyme to desired cut site for cleavage. The sgRNA is a synthetic RNA chimera created by fusing crRNA with tracrRNA. The guide sequence, located at the 5′ end of the sgRNA, confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Cas9 expression plasmids for use in the methods of the invention can be constructed as described in the art. The ZFN, TALENs, and engineered meganucleases methods require more complex design and protein engineering to bind the DNA sequence to enable editing. For this reason, the CRISPR/Cas mediated system has become the method of choice for genome editing.

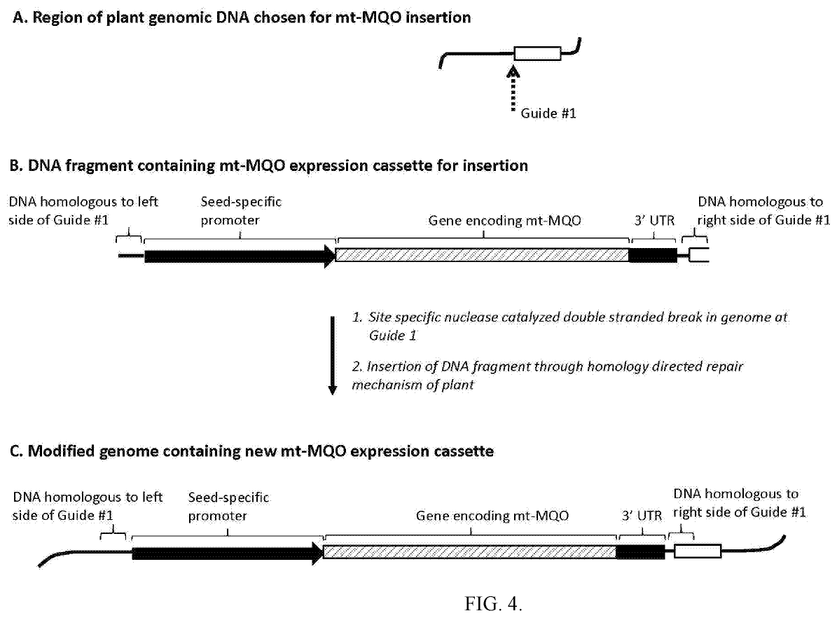

The CRISPR/Cas technology, or other methods for genome editing, can be used to insert an expression cassette for mt-MQO into the genome of plants at a defined site using the plants homologous directed repair mechanism (FIG. 4). A sgRNA with a guide sequence for the genomic location of interest (for example Guide #1, FIG. 4) is used to enable the Cas enzyme, or other CRISPR nuclease, to produce a double stranded break in the genome. An expression cassette containing a seed specific promoter, the mt-MQO gene, and an appropriate 3′ UTR sequence is flanked by sequences with homology to the upstream and downstream region of the sgRNA cut site. This expression cassette is inserted into the double stranded break in genomic DNA using the homologous directed repair mechanism of the plant.

There are many variations of the CRISPR/Cas system that can be used for this technology including the use of wild-type Cas9 from Streptococcus pyogenes (Type II Cas) (Barakate & Stephens, 2016, Frontiers in Plant Science, 7, 765; Bortesi & Fischer, 2015, Biotechnology Advances 5, 33, 41; Cong et al., 2013, Science, 339, 819; Rani et al., 2016, Biotechnology Letters, 1-16; Tsai et al., 2015, Nature biotechnology, 33, 187), the use of a Tru-gRNA/Cas9 in which off-target mutations were significantly decreased (Fu et al., 2014, Nature Biotechnology, 32, 279; Osakabe et al., 2016, Scientific Reports, 6, 26685; Smith et al., 2016, Genome Biology, 17, 1; Zhang et al., 2016, Scientific Reports, 6, 28566), a high specificity Cas9 (mutated S. pyogenes Cas9) with little to no off target activity (Kleinstiver et al., 2016, Nature 529, 490; Slaymaker et al., 2016, Science, 351, 84), the Type I and Type III Cas Systems in which multiple Cas protein need to be expressed to achieve editing (Li et al., 2016, Nucleic Acids Research, 44:e34; Luo et al., 2015, Nucleic Acids Research, 43, 674), the Type V Cas system using the Cpf1 enzyme (Kim et al., 2016, Nature Biotechnology, 34, 863; Toth et al., 2016, Biology Direct, 11, 46; Zetsche et al., 2015, Cell, 163, 759), DNA-guided editing using the NgAgo Agronaute enzyme from Natronobacterium gregoryi that employs guide DNA (Xu et al., 2016, Genome Biology, 17, 186), and the use of a two vector system in which Cas9 and gRNA expression cassettes are carried on separate vectors (Cong et al., 2013, Science, 339, 819).

It will be apparent to those skilled in the art that any of the CRISPR enzymes can be used for generating the double stranded breaks necessary for promoter excision in this example. There is ongoing work to discover new variants of CRISPR enzymes which, when discovered, can also be used to generate the double stranded breaks around the native promoters of the mitochondrial transporter proteins.

It will be apparent to those skilled in the art that any of the site directed nuclease cleavage systems can be used to generate the double stranded break in genomic DNA can be used insert the expression cassette for mt-MQO in this example. REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “YTEN-61543WO-Sequence-Listing_ST25.txt”, created Oct. 8, 2019, file size of 122,880 bytes, is hereby incorporated by reference.

1. A genetically engineered plant that expresses a quinone-utilizing malate dehydrogenase, the genetically engineered plant comprising a modified gene for the quinone-utilizing malate dehydrogenase, wherein:
the modified gene comprises (i) a promoter and (ii) a nucleic acid sequence encoding the quinone-utilizing malate dehydrogenase;
the promoter is non-cognate with respect to the nucleic acid sequence encoding the quinone-utilizing malate dehydrogenase; and
the modified gene is configured such that transcription of the nucleic acid sequence is initiated from the promoter and results in expression of the quinone-utilizing malate dehydrogenase.

2. The genetically engineered plant according to claim 1, wherein the quinone-utilizing malate dehydrogenase is characterized as EC 1.1.5.4.

3. The genetically engineered plant according to claim 1, wherein the quinone-utilizing malate dehydrogenase converts malate to oxaloacetate.

4. The genetically engineered plant according to claim 1, wherein the quinone-utilizing malate dehydrogenase has at least 30% or higher sequence identity to one or more of the following:
(1) Corynebacterium glutamicum quinone-utilizing malate dehydrogenase of SEQ ID NO: 2,
(2) Escherichia coli quinone-utilizing malate dehydrogenase of SEQ ID NO: 3,
(3) Helicobacter pylori quinone-utilizing malate dehydrogenase of SEQ ID NO: 4, or
(4) Mycobacterium phlei quinone-utilizing malate dehydrogenase of SEQ ID NO: 5.

5. The genetically engineered plant according to claim 4, wherein the quinone-utilizing malate dehydrogenase has at least 30% or higher sequence identity to Corynebacterium glutamicum quinone-utilizing malate dehydrogenase of SEQ ID NO: 2.

6. The genetically engineered plant according to claim 5, wherein the quinone-utilizing malate dehydrogenase comprises Corynebacterium glutamicum quinone-utilizing malate dehydrogenase of SEQ ID NO: 2.

7. The genetically engineered plant according to claim 1, wherein the quinone-utilizing malate dehydrogenase has at least 30% or higher sequence identity to one or more of the following:
(1) Solanum commersonii quinone-utilizing malate dehydrogenase of Genbank accession number JXZD01234700.1,
(2) Ipomoea batatas quinone-utilizing malate dehydrogenase of Genbank accession number FLTB01001391.1,
(3) Brassica oleracea quinone-utilizing malate dehydrogenase of Genbank accession number AOIX01037258.1,
(4) Thlaspi arvense quinone-utilizing malate dehydrogenase of Genbank accession number AZNP01005833.1,
(5) Eleusine coracana quinone-utilizing malate dehydrogenase of Genbank accession number LXGH01418531.1,
(6) Tectona grandis quinone-utilizing malate dehydrogenase of Genbank accession number GFGL01159055.1,
(7) Triticum urartu quinone-utilizing malate dehydrogenase of Genbank accession number AOTI011454468.1,
(8) Sesamum indicum quinone-utilizing malate dehydrogenase of Genbank accession number MBSK01001494.1,
(9) Humulus lupulus quinone-utilizing malate dehydrogenase of Genbank accession number BBPC01185947.1,
(10) Arachis duranensis quinone-utilizing malate dehydrogenase of Genbank accession number MAMN01020206.1,
(11) Zea mays quinone-utilizing malate dehydrogenase of Genbank accession number LMVA01099495.1,
(12) Corchorus olitorius quinone-utilizing malate dehydrogenase of Genbank accession number LLWS01002081.1,
(13) Spinacia oleracea quinone-utilizing malate dehydrogenase of Genbank accession number AYZVO2003660.1,
(14) Oryza sativa quinone-utilizing malate dehydrogenase of Genbank accession number GFYC01000193.1,
(15) Ensete ventricosum quinone-utilizing malate dehydrogenase of Genbank accession number MKKS01000001.1,
(16) Zea mays quinone-utilizing malate dehydrogenase of Genbank accession number OCSP01000026.1,
(17) Cajanus cajan quinone-utilizing malate dehydrogenase of Genbank accession number AFSP02228873.1,
(18) Coffea canephora quinone-utilizing malate dehydrogenase of Genbank accession number CBUE020014129.1,
(19) Oryza sativa quinone-utilizing malate dehydrogenase of Genbank accession number AACV01031296.1,
(20) Dorcoceras hygrometricum quinone-utilizing malate dehydrogenase of Genbank accession number LVEL01210429.1,
(21) Ricinus communis quinone-utilizing malate dehydrogenase of Genbank accession number AASG02035827.1,
(22) Arabis nordmanniana quinone-utilizing malate dehydrogenase of Genbank accession number LNCG01168830.1,
(23) Suaeda salsa quinone-utilizing malate dehydrogenase of Genbank accession number GFUM01022853.1,
(24) Fragaria nipponica quinone-utilizing malate dehydrogenase of Genbank accession number BATV01204972.1,
(25) Pseudotsuga menziesii quinone-utilizing malate dehydrogenase of Genbank accession number LPNX010033709.1,
(26) Oryza sativa quinone-utilizing malate dehydrogenase of Genbank accession number AAAA02041020.1,
(27) Syzygium luehmannii quinone-utilizing malate dehydrogenase of Genbank accession number GFHM01044391.1,
(28) Castanea mollissima quinone-utilizing malate dehydrogenase of Genbank accession number JRKL01150921.1,
(29) Cicer arietinum quinone-utilizing malate dehydrogenase of Genbank accession number AHII02009088.1, or
(30) Boehmeria nivea quinone-utilizing malate dehydrogenase of Genbank accession number NHTU01053079.1.

8. The genetically engineered plant according to claim 1, wherein the promoter comprises one or more of a constitutive promoter, a seed-specific promoter, or a seed-preferred promoter.

9. The genetically engineered plant according to claim 1, wherein the genetically modified plant exhibits modulated expression of the quinone-utilizing malate dehydrogenase relative to a reference plant that does not include the modified gene.

10. The genetically engineered plant according to claim 1, wherein the genetically modified plant exhibits increased expression of the quinone-utilizing malate dehydrogenase relative to a reference plant that does not include the modified gene.

11. The genetically engineered plant according to claim 1, wherein the genetically modified plant exhibits increased expression of the quinone-utilizing malate dehydrogenase in mitochondria of cells of the genetically modified plant relative to a reference plant that does not include the modified gene.

12. The genetically engineered plant according to claim 1, wherein the modified gene further comprises a nucleic acid sequence encoding a mitochondrial targeting sequence and is further configured such that the quinone-utilizing malate dehydrogenase comprises an N-terminal mitochondrial targeting signal.

13. The genetically engineered plant according to claim 1, wherein the genetically engineered plant has one or more characteristics selected from higher performance and/or seed, fruit or tuber yield relative to a reference plant that does not include the modified gene.

14. The genetically engineered plant according to claim 13, wherein the one or more characteristics are increased by 10% or higher relative to a reference plant that does not include the modified gene.

15. The genetically engineered plant according to claim 1, wherein the genetically engineered plant comprises one or more of maize, wheat, oat, barley, soybean, canola, rapeseed, Brassica rapa, Brassica carinata, Brassica juncea, sunflower, safflower, oil palm, millet, sorghum, potato, lentil, chickpea, pea, pulse, bean, tomato, potato, or rice.

16. The genetically engineered plant according to claim 1, wherein the genetically engineered plant comprises one or more of camelina, Brassica species, Brassica napus (canola), Brassica rapa, Brassica juncea, Brassica carinata, crambe, soybean, sunflower, safflower, oil palm, flax, or cotton.

17. A method for producing the genetically modified plant of claim 1, the method comprising introducing the modified gene into a plant, thereby obtaining the genetically modified plant.

18. The method according to claim 17, wherein the quinone-utilizing malate dehydrogenase is characterized as EC 1.1.5.4.

19. (canceled)

20. The method according to claim 17, wherein the quinone-utilizing malate dehydrogenase has at least 30% or higher sequence identity to one or more of the following:
(1) Corynebacterium glutamicum quinone-utilizing malate dehydrogenase of SEQ ID NO: 2,
(2) Escherichia coli quinone-utilizing malate dehydrogenase of SEQ ID NO: 3,
(3) Helicobacter pylori quinone-utilizing malate dehydrogenase of SEQ ID NO: 4, or
(4) Mycobacterium phlei quinone-utilizing malate dehydrogenase of SEQ ID NO: 5.

21. (canceled)

22. (canceled)

23. The method according to claim 17, wherein the quinone-utilizing malate dehydrogenase has at least 30% or higher sequence identity to one or more of the following:
(1) Solanum commersonii quinone-utilizing malate dehydrogenase of Genbank accession number JXZD01234700.1,
(2) Ipomoea batatas quinone-utilizing malate dehydrogenase of Genbank accession number FLTB01001391.1,
(3) Brassica oleracea quinone-utilizing malate dehydrogenase of Genbank accession number AOIX01037258.1,
(4) Thlaspi arvense quinone-utilizing malate dehydrogenase of Genbank accession number AZNP01005833.1,
(5) Eleusine coracana quinone-utilizing malate dehydrogenase of Genbank accession number LXGH01418531.1,
(6) Tectona grandis quinone-utilizing malate dehydrogenase of Genbank accession number GFGL01159055.1,
(7) Triticum urartu quinone-utilizing malate dehydrogenase of Genbank accession number AOTIO11454468.1,
(8) Sesamum indicum quinone-utilizing malate dehydrogenase of Genbank accession number MBSK01001494.1,
(9) Humulus lupulus quinone-utilizing malate dehydrogenase of Genbank accession number BBPC01185947.1,
(10) Arachis duranensis quinone-utilizing malate dehydrogenase of Genbank accession number MAMN01020206.1,
(11) Zea mays quinone-utilizing malate dehydrogenase of Genbank accession number LMVA01099495.1,
(12) Corchorus olitorius quinone-utilizing malate dehydrogenase of Genbank accession number LLWS01002081.1,
(13) Spinacia oleracea quinone-utilizing malate dehydrogenase of Genbank accession number AYZVO2003660.1,
(14) Oryza sativa quinone-utilizing malate dehydrogenase of Genbank accession number GFYC01000193.1,
(15) Ensete ventricosum quinone-utilizing malate dehydrogenase of Genbank accession number MKKS01000001.1,
(16) Zea mays quinone-utilizing malate dehydrogenase of Genbank accession number OCSP01000026.1,
(17) Cajanus cajan quinone-utilizing malate dehydrogenase of Genbank accession number AFSP02228873.1,
(18) Coffea canephora quinone-utilizing malate dehydrogenase of Genbank accession number CBUE020014129.1,
(19) Oryza sativa quinone-utilizing malate dehydrogenase of Genbank accession number AACV01031296.1,
(20) Dorcoceras hygrometricum quinone-utilizing malate dehydrogenase of Genbank accession number LVEL01210429.1,
(21) Ricinus communis quinone-utilizing malate dehydrogenase of Genbank accession number AASG02035827.1,
(22) Arabis nordmanniana quinone-utilizing malate dehydrogenase of Genbank accession number LNCG01168830.1,
(23) Suaeda salsa quinone-utilizing malate dehydrogenase of Genbank accession number GFUM01022853.1,
(24) Fragaria nipponica quinone-utilizing malate dehydrogenase of Genbank accession number BATV01204972.1,
(25) Pseudotsuga menziesii quinone-utilizing malate dehydrogenase of Genbank accession number LPNX010033709.1,
(26) Oryza sativa quinone-utilizing malate dehydrogenase of Genbank accession number AAAA02041020.1,
(27) Syzygium luehmannii quinone-utilizing malate dehydrogenase of Genbank accession number GFHM01044391.1,
(28) Castanea mollissima quinone-utilizing malate dehydrogenase of Genbank accession number JRKL01150921.1,
(29) Cicer arietinum quinone-utilizing malate dehydrogenase of Genbank accession number AHII02009088.1, or
(30) Boehmeria nivea quinone-utilizing malate dehydrogenase of Genbank accession number NHTU01053079.1.

24-32. (canceled)


SEQUENCE LISTING (52 sequences)

SEQ ID NO: 1 - protein, 328 aa, Corynebacterium glutamicum
MNSPQNVSTKKVTVTGAAGQISYSLLWRIANGEVFGTDTPVELKLLEIPQALGGAEGVAMELLDSAFPLLRNITITADANEAFDGANAAFLVGAKPRGKGEERADLLANNGKIFGPQGKAINDNAADDIRVLVVGNPANTNALIASAAAPDVPASRFNAMMRLDHNRAISQLATKLGRGSAEFNNIVVWGNHSATQFPDITYATVGGEKVTDLVDHDWYVEEFIPRVANRGAEIIEVRGKSSAASAASSAIDHMRDWVQGTEAWSSAAIPSTGGYGIPEGIFVGLPTVSRNGEWEIVEGLEISDFQRARIDANAQELQAEREAVRDLL

SEQ ID NO: 2 - protein, 500 aa, Corynebacterium glutamicum
MSDSPKNAPRITDEADVVLIGAGIMSSTLGAMLRQLEPSWTQIVFERLDGPAQESSSPWNNAGTGHSALCELNYTPEVKGKVEIAKAVGINEKFQVSRQFWSHLVEEGVLSDPKEFINPVPHVSFGQGADQVAYIKARYEALKDHPLFQGMTYADDEATFTEKLPLMAKGRDFSDPVAISWIDEGTDINYGAQTKQYLDAAEVEGTEIRYGHEVKSIKADGAKWIVTVKNVHTGDTKTIKANFVFVGAGGYALDLLRSAGIPQVKGFAGFPVSGLWLRCTNEELIEQHAAKVYGKASVGAPPMSVPHLDTRVIEGEKGLLFGPYGGWTPKFLKEGSYLDLFKSIRPDNIPSYLGVAAQEFDLTKYLVTEVLKDQDKRMDALREYMPEAQNGDWETIVAGQRVQVIKPAGFPKFGSLEFGTTLINNSEGTIAGLLGASPGASIAPSAMIELLERCFGDRMIEWGDKLKDMIPSYGKKLASEPALFEQQWARTQKTLKLEEA

SEQ ID NO: 3 - protein, 548 aa, Escherichia coli [sequence omitted]

SEQ ID NO: 4 - protein, 450 aa, Helicobacter pylori
MSMEFDAVIIGGGVSGCATFYTLSEYSSLKRVAIVEKCSKLAQISSSAKANSQTIHDGSIETNYTPEKAKKVRLSAYKTRQYALNKGLQNEVIFETQKMAIGVGDEECEFMKKRYESFKEIFVGLEEFDKQKIKELEPNVILGANGIDRHENIIGHGYRKDWSTMNFAKLSENFVEEALKLKPNNQVFLNFKVKKIEKRNDTYAVISEDAEEVYAKFVLVNAGSYALPLAQSMGYGLDLGCLPVAGSFYFVPDLLRGKVYTVQNPKLPFAAVHGDPDAVIKGKTRIGPTALTMPKLERNKCWLKGISLELLKMDLNKDVFKIAFDLMSDKEIRNYVFKNMVFELPIIGKRKFLKDAQKIIPSLSLEDLEYAHGFGEVRPQVLDRTKRKLELGEKKICTHKGITFNMTPSPGATSCLQNALVDSQEIAAYLGESFELERFYKDLSPEELEN

SEQ ID NO: 5 - protein, 505 aa, Mycobacterium phlei
MTRKDAGAVSVAKTDVVLIGAGIMSATLGALLKQLEPNWSITLIERLDGAAAESSDPWNNAGTGHSALCELNYTPQNADGSVDISKAIKVNEQFQISRQFWAYAVENGLVGDPRSFLNPVPHASYVRGADNVAYLRRRYDALAGNPLFGGMEFIDDEAEFARRLPLMAEGRDFREPVALSWAPQGTDVNFGSLSRQLIGYVAQQGMTTRFGHEVRDLDRNSDGTWTVKAVNLRTGRATKINTRFVFVGAGGGALILLQKAGLQEAKGFGGFPVSGKFLRTDVPALTGAHKAKVYGQPPVDAPPMSVPHLDARVINGKPWLMFGPFAGWSPKFLKHGDVLDLPESVTLNNLPYMANVGLTQFSLVKYLVGQLMLSDSDRVESLREFAPTAKESDWEVCVAGQRVQVISGSNGKGSLDFGTTVVTGADGTMAGLLGASPGASTAVTAMLDVLERCFPSRYVTWLPKIRDMVPSLGHELSNEPALFDEIWSHGSKVLRLDEPAAVAVQ

SEQ ID NO: 6 - protein, 85 aa, Arabidopsis thaliana
MLRTVSCLASRSSSSLFFRFFRQFPRSYMSLTSSTAALRVPSRNLRRISSPSVAGRRLLLRRGLRIPSAAVRSVNGQFSRLSVRA

SEQ ID NO: 7 - protein, 25 aa, Saccharomyces cerevisiae
MLSLRQSIRFFKPATRTLCSSRYLL

SEQ ID NO: 8 - protein, 78 aa, Arabidopsis thaliana
MYLTASSSASSSIIRAASSRSSSLFSFRSVLSPSVSSTSPSSLLARRSFGTISPAFRRWSHSFHSKPSPFRFTSQIRA

SEQ ID NO: 9 - protein, 19 aa, Saccharomyces cerevisiae
MLSARSAIKRPIVRGLATV

SEQ ID NO: 10 - protein, 77 aa, Arabidopsis thaliana
MAMAVFRREGRRLLPSIAARPIAAIRSPLSSDQEEGLLGVRSISTQVVRNRMKSVKNIQKITKAMKMVAASKLRAVQ

SEQ ID NO: 11 - DNA, 770 bp, Cauliflower mosaic virus
agagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcg

SEQ ID NO: 12 - DNA, 1148 bp, Glycine max
gcaacagaagacccaaaactcaaaaaagttagtttcgggccaacatttcctcttgagggatgacacgtgacctgctactctggcccttatctggcatgtccatccttcttggcgcgacatttaattcgtcgtcagaaataactgaaggacaccttgcttgtttctcttttggccgccaccggtcttgtcatcgtcgaaggcgcccttgcgcttgtcggcagaacctttttcggcgacctccttgccttttcctttggccttgttcgtcatttctacagagaatgcaatgagaccaacgccaattgcatggttagagttagagaaatggagagaggaagaagtgcgtgactagagtgtgtgtaactgtgaagaacgacgagtccaaaatgaattttactgtaaataatttgaggaaaaaagtgatcaatacatatcatgcggtgcatacaagaatcggccattggtcaacttgtgagaggaaaaaatcatttaactaataccaaataatcttaaaattaataaaataatttaactaattaacccacggaagaaccttcttccgttgactctggcggaagaagttcttccgcatagttccatggaagatggttcttccgcagttcttctttcgttgacactcgcggaagaaatgttccacgggcgtccgcggaagaactttcttccgcaaagctaaagagcatttttgccatgtcgaaatcatcgccaatgaccagggtaacagaaccacgccctcttatgttggtttcaccgattcagagcgtttgatcggtgatgccgccaagaatcaggtcgccatgaaccccgtcaacaccgtcttcggtaagatccctagccgacacttcgccttttcaggatttgcattgttcctagatttttggatctgttgtttgaaactccacttttctattttggtaatttttagttttattttgtaatcctgctgtttatatgtcttattgttattattaatcgttgcatggtctgaactggtttagaactctacttgtattgtttgttaaaatcttatttgaaatcgaatagtaatataattttaatcgaatggtgatatgcataaacatcgtatttgttcgtcgaattctggttttgaattgaataatattgttatg

SEQ ID NO: 13 - DNA, 1378 bp, Glycine max
ctagaaattaaatgtttttaacaggtaatttgagaaaaatgtacttcaaaataattagttttaccagtttatgtcttctttttctcttttttatctttattctatgtttcaaattctaataatacatcatttaaatatttttaatttaaaagtgcttactaaattttaaaaaaatcatatttatcaaataacttctactttaaatttaaacttcattatttttaacttaaaaataacttttaaattaaaaaaatgaaaacaaacactacctaaaccctaaacactatctatctaagtcacattacttaatgattcttaatttatgttctttgtaaactttcatttcttcctccttttggctatacatgttcatttctgtgtactttactatattattagtaaaagccttttatataggtatatcaaatcaaataattaatataatatataattctcttaatttcatttcttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaag

SEQ ID NO: 14 - DNA, 1500 bp, Glycine max
gatcctcacaaacctcacttggagacataggtgtgagggtaacctttttccctttatgtacaaatgaaaatttgtttgtgacaccattatggacaacatccttacactactaaaaaagcttttttttacgacatcatatttacgacagtcatacaaaaacgtcttagtatgtataaggatggcaatttcgtaaatatttcaaacatttcaaaggcagtttcagaaaaccgtctttgaatgcggccattttaatttttaacgcgcccctcgcatccgttcctcttctttccgcaaatgtggtgctcgttccttttctttcccagctggcatctgttcctctccccactcgctagctatcttctgcttctcctcttctctcctcttcccattacatttctccaccttctccctggtaccaccaccgccccccactccacattcgtcctccgcccccattcccctatcctccagtaaaattacaaaaaaccctaacaccaaaaaaacccaaacccctgtcgcaatgaaatctccacccccaaatagctctttggaatagaatcaaggaacttaccaaatccattatatgctattggggttttggcatgtttccggtgtgaaagaaggaaaaagaaatgcgtatgcgatggtgatgtacgtaggtacgccgaaggactacgaattctacatagccatactcgtgcttctcaaatcgctggctacgctcgacgttgaaattgatcttgctgtgattgcttcccttgatgttcctcctcgatggattcgagctctgtaagtctcactccttcaccatcatttgccactttatttttatgtacttttactttattattatttgtaacctgtatttttatttggtttcggatatctgttgctttattattcaccctggaatttggttgattttattatttttgaaaaataaggaaagagatttatttgttagcttaattgttttaattggcgaatatgtttttcttttcccttttttgcacagagtgaagctttgttcttagggtaatggattcccttttttgtgatgctagtggatgatttgactgattagtgtttagtggaatgaagaaccagaactagtagtaggtagagggaatcacttttggttttggatgtaaacttagaaatgtgcagcactgcacagaattgatatttgatcgtgggtcaaattgtcaaaatgtgcaaagaatacaaaggcacaggtgatatcattccattttacgttttttaacgaagctgttagtttcaattcaattatttacatatataataaatatattgatacttgctttagtttcatgaattaaaagaatttgattttgtaaatttcatttgaatttgtttttgtacaagctctcaacttttattatatgaacgagaagtttcttttttcctttttgagtttatttgaacttgtggtgttctaattgtatatatttttgtgcaggtgtcaatcggtactactac

SEQ ID NO: 15 - DNA, 1261 bp, Glycine max
atctctcgacagttgcgaactgaacgctgagttggtaatgctatgccctatcgctttttgcaccgtcccatgatcatttcccccacaccaccccatcaacctctaaaaagttaagagtgaaaattacacacacccgaggagaagaaaagctgcttcttctaagcatcacaacctagttactttacttgtagggccttttccatttcccctaaattacccctcttttcatcatatgataataatatccagctcagactatagtatgatattatgatgtcagcataataggttggcactaaagtcttaaagggcattgtacatgttgcacctggcattcaaattcataaatactaacactgtgaaatagattataaatcctcaaataaatgtcacacggttggggttcgaatccactcaaaaaggctaatgggatgggatttaagtgccaaggaatataccatggactttaacagcaacacaatttacaatctaaaatgtattacttttttttttcaaaaaagatatacaaaataaggtaccaagaataaaaggagtatttagaaacagtggcaccaatttaataaattatttatataaaatgacacttatttaatttatcaatgataaaagtaatattgatttattctctgattaactgttcaattaatagtgttattatcataatctgtcgcaaaagttatttttatcaacaacaataattgatacaagtagtataaaattaagcctcttagttaatatagactacttgatactaaaaccatgttacaccaaaaagtaatttttatgtcacttgtctatataataattacgactaaattaataatttttaaaaatattactgaatccattaaccgaacttttataatgaaagtatttttatgctttaaaatcacaaacattgaataaactaaaaatgataccacggaattggaacaagagacgttccacacaaaagaaaaaaatatgttgaataattgaaacggtgacaagaaaagtggaataataatacaaagatggcagatggggttattgttattggaggagatgagtgaaataatgagtgaggggggtgtaactggaaagcaagaaaaagcgcaagagtgccagctatttccaacaacaaacgtggcccgtgggatgcgatattcgtaacgaacggcgaggatggaaggacgtgcaatttgcgcttcatttgaggcgaatttcatttggccagaccttccttttttaaaccacaggg

SEQ ID NO: 16 - DNA, 1094 bp, Glycine max
tgtgtcaatgttgtttctggtgaattgacataatgaattctacctgtacggagtagagaataactatttacccaacaagaatgattatctcattaatttttgaagtagacgcaataacgaatatattatacattcagaaaaatttcaccatattattctcaaatcacaacaataatttgttttttttttgcttgatataaaaccaatactctatactttttaaggttaatttaaacttaaagagtatttttaagatgcatgtactttaaggaataatagaaacatgacaacatcataaaagaatgaagaaactgaatcataacgtagtttgttacgccttccatttggtggttgatttggatacaatctagattggtttgctaaatggtttataagttatgtagacgtttttattactactattttagacaaatcaaatacacaccttcactttattctattcaaataacatgatttttcctaacattttttaaaaaaattactttttaaatataaactaattattttagaaatagttttataaaaatccacgccaaaaaaattaagttgtttttataaatataaacatcgggcttcaatcttaaatttataaatgtacgaaataatttgacagttaaatggaaattgctagcatggaagtgtttttatcatttatcaaactcaaccaaactgaacatcagaataattattagtgacaaattttgcagcatatgaagtggcttgcatagctccaaggctggcgatcatatgtcagattagagcaggctctctttggtactatgatacatttcaagcaaataacaaccgtaaaaattcacgccaaaatttttggaacgaatctatatattattattttatttcttttgatttcatgtacgtacagtgcccgtaattgacatgtctttgttccttaatgcctttcccacgtggaacaggcacctagaaacttggactaagtagggaattgagggccatggactatagtgccaaaccaacatcattttatatatatatatatatatatatatatatatgctattgttttctatagtttttggaaattaatacttatc

SEQ ID NO: 17 - DNA, 1449 bp, Glycine max
atttgtactaaaaaaaaatatgtagattaaattaaactccaattttaattggagaacaatacaaacaacacttaaaacctgtaattaatttttcttctttttaaaagtggttcaacaacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttc

SEQ ID NO: 18 - DNA, 1321 bp, Glycine max
aaaaacacaaaaaaaaattatacaaaaatgtttctcacaacatgagaagtaaaatccctcaaagaatttcacatcatcatatcagaatcaaaggaatcaaaatcataggtcaaaaatacaaaaacaccaagaacactcaatttattaactaatttgcatcatgacatcaattggtccatcaaacacaacaatcttgtaattataatcgtaacgaaagaattacaatgcaataaacatcccaaaataaacctcaatttaatcctctaaggatccctatacatgttcattctaaccccaattgtgataaattcatcccttacctctaagcaggctcacgtgtgtagtctggcagtgatagaggcatctctagtggttttctaatagtcctcaagcttgtttttcctctagttgttctgttaggattttcaagcgttagagagaagaagaagagattggagcctctatttcactgttaccgtacaagggatatttttctcaccataaacattattttgcaaatcccaacgaaggagatgtccgtacataagttcgaaacctggtgctcgaatttcacgacgattcaatggttaacaagtccaagattgtatttttactgtgacagatttgagtgtatacaagaaaaagagagctccatgcgaggaatatttctctcacagtagacattatttcataaatcccaatggtaaaaatatgcaaaaatgagtttcaaacctgcttttaaaatttcatgacgactcaacggttaacgtgtccgggattatattttcactggaacaagtttgagtgcatgcgggaaaagagagggttttgggagaggaaaaaaggaaaacaaatttaagaggaagagagagcgtaaaaatttatcgtaaatgtaaaaaatgacctaatatatctctatttataactagggtactctcaatctattatttactcatttttttattttattattttataaaaaagaattttattttacttcctatcaaattaataaataaaacattcttcttattttctaagatcacatatttattttatttaccttaaaatcatcattttaattaataaaattatttcttcttatttatttaattacaaaaatcttattatttttttaaaattttatttatttttaaataaaatattttttaatttattttataaaaaatgagatgttacattgaattataaaataaatagccaacaataaatagccgacttgcttttgcattgactaaggaagtcaagtcatcaataaatataatttccagttggcaatattctcaaagttggtctatat

SEQ ID NO: 19 - DNA, 514 bp, Glycine max
agatttgatcgatacttcattaaattgacattttattttaacacataatacattattaaaaatataaataaacatttacagcgaagttatataattaaaagcctggtctatgtaatggtaggaaatttgaaaatctaaaagcaaacaaaaattgttgtttatggtgctaagttgcacctggaaagatgcattgtttagctaaaacattcacgtcgagtacttggtttgggaaaaaaagccattcaagcttagctggtcctctctcctgtctctctctctctgtctgtctctctctgtctgtctctctctcaagcacatacacaaacaaagtaagggctataaataggagggatggaagtggaagaaagtctatagcgaagtttcatttctttggattagaaatttttcccaaagctgatcgagaagccagccaggccaggtctgtagttttctttttttctttttaatattaattcattattgtgttcttcatcatataatataattaagcctt

SEQ ID NO: 20 - DNA, 702 bp, Glycine max
cgcgccgtacgtaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgcggccgc

SEQ ID NO: 21 - DNA, 579 bp, Glycine max
acgcgccgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgc

SEQ ID NO: 22 - DNA, 1500 bp, Oryza sativa
gcagctgttttcgcggtacagggtgcaacaaaagcccatgacggcccacacctgcctctctccgctccaaacaccgaaacaagggggtgggtgcaatgggccggcgctcgaagaccgcgaactctttccaacagcccagcgcattagcccctcctcctactctctctaccttctttttaacatgcgactttctttctgtggacgacggcatcaacgacgggagcaggagcgggggctgaagcacggtgcgtgggctcctggagtggcgacggcctctccggcgagcttcctctggcgaactccctccgctcctcctatggcgaaatccaaacaagggtcagtttcgactccaaccttctcccaccaccacctcctgaccgtgccaccacccggccttgtcggcactgaaaggcgtcaacttgtcagcgcgggcctgctcggtcggtctcctcctcccctatttcgtttagctttgcccccgccaccaacaccggcccacggcccatggccgaccccgcggctttggcgccgccatcgctatctcgccgctgtccttttttcatgaccttcggtgccatccctctaaattcgatgcacctccctggctctatctccctttacctccgaaatcctaaccctacccataatctctagtgagtcttgtctttatttatggcctctttgaatcgcaggattgataaaacgtaggattttgataggaatgtaagtgtaaaacacatgattgtaaaatagaggaaaaacataggaatggccgtttgattgaaccgcagaaaaaacacaggaattagatgagagagatagactcaaagttactaagagattgaagcttttgctaaatttcctccaaaatctctataggattggccattccatagaaatttcaaaagatttaataggattcaatcctttgtttcaaaaaacttcatagaaaatttttctatagaattaaaatcctctaaaattcctatgttttttctccaattcaaaggggcccttaggttggaatttggaaagtgttcgcgagaaatcaagcggtcgcacgttagcgaattaggatttccggaaacaaaggaccgactccgcctatccatcgtcacgagcacagtgtagaacctcccagacctcaagagaccgttcaaaaagcgcgcgcccaagcggggcccaccaacgcgtccccaccgtgtcgcctcctgattggttgtcccctcttcctttcacgcgaaccggcaccctcccgacccttccagaacccccaatccgacggccaggatcgcccgcgcgcgaacgttctagacccccgccacctccgccacaaaacctctgcccctcccctctccccccgcttcgtctcgttcgagaaatcagaaagagagagaaattcccacgcagcagcaagcaatccaatccgagagcgcgcgtttgcgattattcgctttcgattccgcgaggtttttggagagggaggagaaggaggaggag

SEQ ID NO: 23 - DNA, 1500 bp, Oryza sativa
acagcatttattgtagtctggtcaagcgtgtcacgctgcatgcaacgcagtacagcgcgttcctttacccggtctgtgaccagtcacagaccggtcagatcacgggttaggtggcgactggcggtctgacgcacgccttgccccatcccgtcaagacgaaagcctctaggcactcgtctcaagccggagctagcgtgttatctcttagagatggcacgttagccctggttagatttataccaggcttcatcctaaccattacaggcaaggtgttacacgaagaagggcaaaacatgcacgttgttaaactgacgcgtgggggacaagaatgaccggtctgacactggtcgcatcagcaacgggcagccacgatcccgcgtcatctccgtctccgccgggagtggaggtaggtgtgggctgtcccatcagaagggctcccggatggaaaccgtaccgatctccgcccattaaagagaaaaagaacagtccagtttggaaagagaagggtgcatgtggtatccccttgaagtataaaaggaggaccttgcccatagagaagggggttgattctttccagattcagagcctagaacgagggagaggtgggctcacactttgtaacttgtccatacacaaatccacaaaaacacaggagtagggtattacgcttccgagcggcccgaacctgtatagatcgtccgtgtctcgcgtttcttgctggctgacgatccttccacatacagagagagagagagcttgggatctcaccctaagcccccggccgaaccggcaaaggggggcctgcgcggtctcccggtgaggagcctcgagctccgtcagacatgttcagtttcattatattatgaaatgtcacgtactgtttgttctagttagtgaattgtcatatggtaagaatatataaaaattaggttttctggactctatcttccaatgtatttttggatcctataacaaaatattttcataaatatattttttaagaatctaaacttttttgaaataaaagagcaacaaagaaaataaaaacgctctctcgtaagtaactcgtgaagatccatcgagagccactcgtttgaatcgtcgacacaaaagaacacttcattgattgcttttcgtcaattagccgcacagcacagtactctccaatctgctaaaccaaaaccaatctcatccatccatacccttcttgacaccaagtggcaactcctgattggacgcgccctatcctacatggcacccccaagattctctcgataggctacaggggccacaccgaccctccacgtcatcgtccacgtcaccctcatcccggcccatccagccaatcccagcccagcaaaaaatcttcccaagtggccaccagataagcctctccacgtattaatacgccaagtgttcgtcgccatgacacagcacgcacacacaccccaccagcagcagcagcagtagctgagcttgaagcagcagagcgaggtagac

SEQ ID NO: 24 - DNA, 1500 bp, Oryza sativa
tccacctctgttggttgcatcgacgtcgcttccctagctcccgtctctagtccggatcctattcctccttggagaccgaagctaccgcaaccattgctcggtggttagcgagcgtggagctgtcctccccactttcgcgtcctcgttcgccaccacagccatacttcgcatggtgatgtcttctccttcactcaccgctaaactcagtgcaaccgtttctaccctagccccggccgccgctctcatagaggtgaaagttcatttacatgtaggtcccacatgttttatgttttttatttttcttttactgattagcatgccacgtaaatcaaaacaacaatccatagtgttttaagtatttttatttaatacgtgagatggagtacaaaaacgagagatgcaaagtgaacttgctaaaacacattttctggttgattacagtcgcttgttgagccattggatcggtcataggattcgtgctagcatacttaattacgcgtaactagttgtgctttataggttacaggtcgctaattagcggtctactggagaactttgctactatttttttcttcactgcatgcactcgatcaagtatgagtatttgtaccgaccagcgaaacacatatgtaattaaagtataaatatgtaattagtatatattagtagtatatttagacagtagttacaccctacatacacaccacttacatatataattagtatgtaattttgtaacttacatatgtaattttagtacttacatatgtaattttgagacttacattgtaaatacactaaaattacatatgtaatttagtaacctacaatgtaaatacatgccgactaacttttgatgaaaaatatggtgttataaatatagctactcccgaactttattccttctctgtgagatatcagtggaaacgctcggtggaatcgggggagtatttgggagcacgcgccgacgcgcgcgtcgtgcgtgccgtcgtctttgtcgcggtggagcggagcgcgcccacttgcgcgcctgggccggaggcgggcgcgccgggggttcgggaatcccctggagccacacgtaaaggcgcgggcgggagggagggaggggccagctaggataaggcacgcgcggccgctgcgattggggcgcttgtgaacaccggggcgccacgtggagaggacgttacactccagccgccaaatttccactcccacacccgcgctcccctcccctctcttttccgtgatcgcacctcgcccacgcgccccccgccacacacaatctctgcagctctccagcttcgttggaactcgcgaatctctctccgatcccaggtaaagcagcgaacgacgtcacgcacgacgctgctcggtggatttcgttccttgctggggaaaaccatgcagagacgaaggtgaatgatctgcttttgtgtacttgcgtttaccaggtgaagcgcgagcttggagttggaggggagatcgatcagggccagg

SEQ ID NO: 25 - DNA, 1500 bp, Oryza sativa
ataattaattaattaatcaatcacttttcgtgctgtaaaaaatctcacccgatttgctgaaacgaactgagccgggcgactgtgatattctttcacgatttctgtttgtggcagtgggacattgctgtttattcgaaacaattttcaagtaaaaaaaaatactcaatggtaaggttgctagtaatagtttaacagtttgtttgcagctcagcaaatttcgtttcctcacagatgacacataactgaaagcactcaatgtaatgttgtgcttagctgctaaagcatgtcacgtcttagaaaacaactactccaccatggagaatttttcctcctacttactcctcacatacttaccatctccatataagttcccttgtcgtatcatatgtcttattcttcttgagcacagttattacagcagattttgtagaatagttatcgcatcaaaattttcctatgtcacctttgatcatgtgttatgtgtgcctcttgagtcttagggttaatgtggttgtaatgtgtttaaaaaactatatgaaagctcgtgtgttgctacgggagagagatacctcgaatgaatgtgagagatctccatttgagttgtgtaccttgagagagtgaaagatcacactatttatagacggttaataatggttactgaggtcgattcaccacatcgtcttaaacatttaatgagcatcctccacgtgaaaagtagagatgatagcgtgtaagagtggttcggccgatatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcataag

SEQ ID NO: 26 - DNA, 1945 bp, Oryza sativa
aaggtttcatgcgtatcgtgacagatgttacataatgacaaattccccagctggagcacctttatccctgctgtttgcatgaaattagcttgtcttgtagttccctccagcaaaaagaagtctgaaacaaaacaacatttcgaaaaaaaggcatccatgagttagcatttctacagttgtctatagaggggaaggctgcacgacaaagtttccaggcttggaaacaacctcttatgtaaaatttttcgtatgtatcagatgatttgtttgcgttacggcatctccacctaacatcaccttcatcatgcgcctatggtctttctcttgcctgttttatacgtaaaattggaaacgacagaaacttttgccatctttattaaaggaaggcaaatatgcaaatataggcatcaagatcacagttagtggattatcatctttgtaggttaacatgtcctaccccaggggagcttatactcaagtactccatgcattttcatgaaatgagaaaaaacgatttttaagagaaatgtactttcttgtatttatgccaaatggcaaggactgaaagggaaaaactaagaaagggaacgttacagtaaggctctgtggggactggggacttcagagaaacgtgaaccctgcttccttcctctgcatgaacataacaccagaggtttccagcctttcacacagttgttgatggcttcacacaattcatctctacctcctgactctttataaggacccccagcatcaccacaattgcacaagtacaggcattagatccacaagaacacttgggcaggcaagcacctctttgatctttaagccgttgttatgttctatttctgagcatatggtttctagttatattctttttcttcattcgtttcatatctttgaagtgttgatgcaaatgcggtgaacaactatcaactgtgtactctccaagtgaatgcgaataatcatttcctgtgagaattgtgggctagataaacgaatgaaatgctgttttatctatgtcatgtgtggaaatttagttaattttccggtctttttatgcattgagatgggtatgctgtttttttagttgggtcccatcatcttgagaattctttcaaatttccttttctttatcctatataaaggatagagaaggcgtatgcctaggtgcaccaaccctgaaagttttattctaattgcgggaatggtttgtaatttttgcttgttcaggttctttttcgtggcctttcttttttttccccttattttgcttagtctttcacagtccaatttttgggaagtagtatatcttagtttggtcctaaggcaccatgttgtactgcaggaaaaaaaagagtaattgtattctgttttttccttgattactatatccctgttttaattaattttgtgcctttgttgtttgatgttggaacttcaatgcccataattagtcatttgacttgttttgggttttgacgctatcttgagtgccataggaaactggtagaatttagtaataattttatatagactgaatgttgagcccaccacaaatggtttccttctgtacaagtatttaataactcaagcacaggaaacatcagatctctaatctaaaggttaacaatgggctcaagcaggagcagtagttcagctctatctgtatatttagaagggctggatctacctgtccaccagcttttaattttaccctggcagctggataacttcttgtctgttaatttcatttagtgctgtgttattttcttcttgttgttcaggatggatgcttttgaatttctggaatttcgtattttgttctatctctttatgaaatgacgttatggcacactttttctgcatattcttgatgaaaataattacctagtcatttttttagttgcaggtttgtctgggactttgagtacccatgcaattc

SEQ ID NO: 27 - DNA, 2315 bp, Oryza sativa
gttcaagatttatttttggtatttaatttacttgcttaagtcagatatattcccatcgttgcaggtttgtcacttagtattattattaagcgctctagcactaggactctggataaataagaaagtttattcacgaggctagagtagtaatcaataacataagcgtggtgtctaggtcagcggttatcttcatatgtagtgtgctccatggaaagtgaggtaggaggaaggtggtgacagtcccgtccgtcctttgtatccctccatgttcgggtatatcatagagctacaggctagacttagcttggcagactaggggagagccggtgctcgaagcaatccatgaggctttacatttaacataagttagtaaattaacccataggaatcatctctagactgaacctaccagtagttgtgcttggatataattatattcctacatatacatacacgttccctgcgattagatacccttggaatactctaaggtgaagtgctacagcggtatccgtgcgcttgcggatttatctgtgaccgtatcaaataccaacaggtagatacaaggaatcatctctcctatccattggtttatcatcttttaaaattatctcttgctctcctattgcctctgcaactgcggataggtgtttctcaacaatgaaggttgtgaagaatgctttgtgcaacaagatggatgacaagtatctcagccatagcctcatttgctttgtagaaaaggatatgtcggacacaatcactaagtatcaccgtggaaaggatgcactgtatgccctatctatatttaccatttagtaatatttatatggcttgtgctaactttatgttgtctttacaggcaataacattatttggaaggcatatctatatattactatttaagataatgtaatatctcaaagtttttataagctgcaatgaggtgagtttcacttagctttctaacttgttatgagttatagatgcatgccaccagtcattttttatcttgcatcagcccctgcctgttagaatatgtttctttgtctgggagtccatgtcaactagccaatttccaaatatatgaacaaaactatgtggcctttgtaacccaaatgagataaagactactctccatagaaatttagcaaacatggcactcaaagaaaatgtgttggatagtttcatcatgcatacaaaagcaacacttttgaactaccattccaaatcctttttgtaaattatctttgcttaacactacccctttgagcaaatgtggctttgtgcggaaaaaactcaaacttggtagggtagacatccatttatataattggatccatgtacataagttgttgagtacttcaagtacttacccttgtgatatacatctcaaatatattgaagaagagaagttctttttttgagagaggttgaagaagagaagtttgtccatagctgaagaggagttttatagtgtctagcttaccttgctgctgattgcatgtctaaaatgtcgtttaatttgggctataatgaaatattcaccaatatttctgctggtctattaaagtttaatagttactcgtaactcatttattttgggctataatttaatattcacctatgtttttgttagtctattttatttccctagtgtgcactagcttaaccccaaattagttttgaacacttaacctaaatgtgtctattatggtcagacactctctcacggcactctaacaaaaagtgaattttgttgttatgtttttgtcatgatctcacaagcaatgtacatgtacgtttctagagtgcaatcttatgctagcctgattgtgaatttagtgtagtttgttttctctttttgtagctacactaccaataacctattgtcctctagtcataccacgtaatcacaaggcaaatccctaactctcacctttaaaagcatgtctttattttcttgggtggcactaatacaaaatctttttcagcattcctatgtgcgatagcaagaaaacatggcataactcttgcttcactctaacaaaaaaaacacttttccaactttaaaacaatggtatctatgtgtttaatgatcaatcaagcatataatgacttacaagtttttacctatgccctttttgcatcatcttgtttgcaacagacaaactagatattcctttaggctataaacacatcagcatgataaagagattaggtaagtttgttatccctttttgcatatattctcgtctactccgtgtatataagcccctctcctccaactcgtccatccatcaccaagagcagtggga

SEQ ID NO: 28 - DNA, 1194 bp, Oryza sativa
ttgcatgccgtcgtcttaagcgtccgcgtgtgaaaatcggattttcgcatacggttgaaccggtcgcatgcaaagatcgcgatcttcgcagacgatttggcacatgcggttgcaccaaccgtatgcgaaaacccttctcgcccgtatgcaaaaaccatctttgttgtagtgtacggttcacaatggtttggatgggaaatcattgtgaaccaaaagtgatagactgatttcgacgagtgtttttttttaagtagtgccacaattttggtcatcatacgtcgtgtctaaaattgtaacttttgaaaaccaatttacattaaattaaatttataagactaaataaagacgatggtcattgaacaattgttgagaaaaatctacacacatgtgtgtccaacacaaatgtttacacatatactactatgttcatagtcgaagttagattttttttttccttaaagggaaagtctgttttcaaattttagacctcactccttccgtttcaaatatatcgtgtatttttttttctagggcaagcttttgaccaatgattactctattatgacacaatgttaaagggatagattcatattcaaaattactattataattataattttgtcatataaataatattttaagcaattgttagccaaaatctcgtcctaacgaaacaaaatacgccttatttttaaaaacacggagtatatccttaaatatttctctatccaatataaaaggtcaatcttttaaaattccgatcatcaataatttctcaaataattactttgaaataaaaaaacatatgcaaatttgtgtcgtcataatatccaatgaacttattcaaatttataaacttattttaattcaaaatttgatcattaattttttttttaaaaaaaaaccaaatcttatcataaacgtcaaatatatttttgatagtgggggcgataataccataaaactaacaacagaagagacatgatactactactgtaatcctaatacgtacgtacgtatacttctacgccggatgcataacttcagccttgtgagacacaacagttgctgcctagctcgtggtcgttggttttttcgctcgagaaaccactacgcgtaaaccgtgaagtatattatatatagccaactggtcttctcgcaaatccgcacatccctttctgcccctcgtcttct

SEQ ID NO: 29 - DNA, 1500 bp, Oryza sativa
gcaaagaaggccagtggcctttgcagctaagctagctagctagcccttcttcctctctttcctgctttccctttgccttctcctattaatcctctgcacctcacacagcagcagaaaacccaccaactggagctctcctttcctactccaagaaacgaaggtagagaaagaaagatcagatcagcttcaggaccaattttagctaggttatatatctctttgcgtgctaatgtgttttagttatctgggtgtgtgtagagttctttgttaaggcactgattcagctgcagtttagattcaagtttgtatgttctctctttgaggaaaagaaacccttttcctgtgcttcgagttcttgcaaagagaaactgtgatgcttggcttccagtttgatgcttctttgttcagattggaaattcttcctagcttctttctctatttatgtagcaaggattctttccggcccagtgatcctggtttcttttggaaggtttcagttttttcgttctttcttgaaatttctcttcttgccttaggcagatctttgatcttgtgaggagacaggagaaaaggaagaagctagtttcctgcggccgacctcttgcttctcactttgtgatgagttttctttggtcaattcttagctagatatgttaagatagttagttaagcaaatcgaaattgctagcttttccatgctttcttaaacatgattcttcagatttggttggttcttttttttcctttttgtggagacgtgctgttcttgcatcttatccttcttgattcatctacccatctggttctttgagctttctttttcgcttcttcccttcattatttcgagcaatctctgcacatctgaaagttttgtttcttgagactacttttgctagatcttgtttactcgatcactctatacttgcatctaggctcctttctaaataggcgatgattgagctttgcttatgtcaaatgatgggatagatattgtcccagtctccaaatttgatccatatccgccaagtctttcatcatctttttctttcttttttatgagcaaaaatcatctttttctttcaaagttcagcttttttctcttgttttacccctctttagctatagctggtttcttattccttttggatttacatgtataaaacatgcttgaatttgttagatcgatcactttatacacatactatgtgaatcacgatctcagatctctcagtatagttgaattcattaatttcttagatcgatcagcgtgtgatgtagtactgtaaatcactactagatctttcatcagtctcttttctgcatctatcaatttctcatgcaagttttagttgtttctttaatccggtctctctctcttttttaatcagctgagagtttgtgctgttctttaatcattaccagatctttcatcagtactctctcttctgcatctatcaaacttctcatgcaatgtttttgctgttctttgatctgatctct

SEQ ID NO: 30 - DNA, 1500 bp, Zea mays
agttttcgcttgtctattcaccctctataggcaactttcaattatgtaatcactttttttttcttttttctgtttaaaatctcagtttcaaacttccaattgattttgaatacgaggtttgggtttaaattcatattggaggcaaaaatcgaaagttccacgtgatgctaggttttatttcggttttctatctcctattgtttttcacgtttcaacttgattcaaattctagttttttttaacttaagcacaattaaatacaacataaaaacaacatggattcaagttctatttcaatttttattaactattatgttgtctagtctgttcaagcacataatacttataaatataaaattaaacgaaatcacatatttccacaaatcttgggtactacactcggagacgacgatggattccatctcaatttggatgttgattatagctctatttcagttgtcactgttgtcctaacacgccctattgtgcatgatagtgcacgtgctcaacgtaaaagaaaagagatcagtaacaagtagcagcactgtacaaggtaagccgtgattcaattaaaactgtttgagcaattcagttgctagatcgttccaccatcgataattcgatatgtacgatgatataaaaagagcccataagtttgtcttgaaaaggttgatcaaataatttaaattagatgataaaaaacatggaagatgtgggagtggacgacggctatgaagaatagtactatatcaggtttatacgtaaaatttatttttgaaatgtttttataatctgtttgaattgtattttttgcttaattatgtgattggatgttttttcatgaaatgtcgagttttattttaaataaaattctgtaaagagaagttgctgcgctgagaaaactataaatcgatagtaaaggctgtacgcaacgtttaagtccttgtttgaatgcgtatgaatctgagaaagttcagaatgattaaatcttttttatttaattttaatttgagagagattaagttctctccaattctctttaatttagacgtaatcgaacaagctggttgccaaactagatgagtacattttgtccactgccatagagccatcgactacaaaagtctagaacacagtggaaagcaccagacaacgcgcgaccaaaagggcccaggccccagcgccccagtccgggggttgtgttcgccgacctgtgcgtgcctgctcgtcacgtcacgtccctatttgcccgtcttcctcccctccagacccttctcgaacgccccttcgttctggatccaacggtcggtctctgccgggctcgaacgttctcgaaaccacgtcacccccgataaaaccccacgcacagcctcctcccttcctcaaccatcattgcaaaagcgaagcaagcaatccgaattctctgcgatttctctagatctcgaccacccctactagttttggttcctcctttcgttcgagagagcgtttctagtggca

SEQ ID NO: 31 - DNA, 1500 bp, Zea mays
caacttacaagcgatgaggccaagacgattagacgaatagctacagaacaagacaatgagagttcagcactcactttttgccagttccttctccttggcagcagccaggcgcttgagtttagcagcttgtgcaaatgtggacggcctacagcagacatacaggcaaagaagcgaggagtaatttgcagttggaaatcattcttcgatcaatagggaaactctgagtcacagcgaaaggaaggttaattgcctacgttgacaactgatcagcctccttgagaagttgcttgatttcaagccgcactttgatctgctcatcactaagtcctccgctctggatgacaaaagcacagaacgcatgagtggcaagtggaaacactagagcgaaataaatacaaaaccgcagactacaggctaacagatagggagaccgggaagacaaagactcgagcctgcattcaacagttacagtcgcctcggccaaaggttgagaaatttgcatcaaaatccaaactgtctagggccatgggaaatagttcctcggaatcagagttcaattcatggacgaaatagatggaactgatggtaggctactcttccgcccaatcagaattcacggaagatccaggtctcgagactaggagacggatgggaggcgcaacgcgcgatggggaggggggcggcgctgacctttctggcgaggtcgaggtagcggtagagcagctgcagcgcggacacgatgaggaagacgaagatagccgccagggacatggtcgccggcggcggcggagcgaggctgagccggtctctccggcctccgatcggcgttaagttggggatcgtaacgtgacgtgtctcctctccacagatcgacacaaccggcctactcgggtgcacgacgccgcgacaagggtgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 32 - DNA, 1500 bp, Zea mays
tctcataaaagcaataaaacaatatctcacaaaatacaagtggcaaacattatacaaacatacacatagtcagaaagtcacaactcaggaccttaaaaaatgaaactatccgattgaaaatacattgataacaattgaacactagaaaataatatcacaaatcaaactatggagcatataactagccatataactcttataatacaataataaaatcatcatatatttaaataaaacactagcaagtctaataacatatgactatagaatcaagatgtgtatgatgacatgacacttgcaattttatcatctcctactactcgacatagtcaatataattgatgtcctccttatctttaaagtttccatgcgaattataaatatatgtatgaagagtaatgattgataagaaactataaataagagtcacaatagttcaaacaactctaaactatatatcattagatagatcttgattttagaaaaataacgaaatcagtttcataattttctaagttaagatgaatttacaaagattagtttagatttaatattttttctgaaaaaataccgatttcggaaacgggcaaaagagatccaaactatttctgtttttttttaccgatttcatttccgtattttcggtaacggtttccggtttcgtatgaccctaaattttggtaaagtttcgaaaaaaaatattttaagaactgaaaattaacgttcctgttttcatccatactaatggctctttaccgctaaaatgttgcccacaatcattgagtaggtttagacgtgagagcaaacagtacaacattacgattcgcccttgcccaaatttacatgccttttccctacggaaacaacatagaatcaagttgacggggttacttacattgaagtggccaaactgatggtagctgtagatttggatgtatgttttctataaattagtcaaaattgagacaaaataaactgcaatttaaaactgaggaaatagtaaaaaaaaggtgaagaagggaggaagaggaaatcagaagcaaaaaatgggcaactttaggcccattatctcgatggtctcgtcggagtccagatatgtgattgacggattggattgggccgtacatcttgcatgagagttcgccaagatttcattgtttaacaagaagcgcgtgacaacaaaaccaagcctatctcatccactctttttttcccttcccacaatggcaagtggcagctcctgattcgctctggccattcctacgtggcacacaccaggattcttgtgtgataggccactgggtcccacccaccaggtgccacatcagacgccaagccatcccggcagaaccaatcccagcccagcaacagatggtctgctatccagttccaactgtataaaagcagctgctgtgttctgttaatggcacagccatcacacgcacgcatacacagcacagagtgaggtaagcatccgaaaaaagctgtgatctgatcgac

SEQ ID NO: 33 - DNA, 1507 bp, Artificial Sequence
  Synthetic construct hybrid maize ubiquitin promoter/maize ubiquitin intron 1
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 34 - DNA, 1996 bp, Artificial Sequence
  Synthethic construct maize ubiquitin promoter/maize ubiquitin intron contstitutive
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctataaagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaaatctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagg

SEQ ID NO: 35 - DNA, 1500 bp, Zea mays
cgagaatatatgttatcttcgtcgttagagaaatctagacagtatacaacaagatccacgtactacaggtaaacttttaggggtattgtgaacaagaggatgagtaaactctaaaagaacaaagctccaatgaaaatttaggtttttatgtggttagtcatagggcaagttgcaaacaggtgttgatctaaaaaggaagtagtagggaaatgtgaagtgtctttgcgaggaattggaaaatgaagatcacattttctttgggtgcatcatgggaagaaccatttgggactcttttaaggaggcctaagaatgccataaagtttgcaagatctttttgaagagtgtctacctataaacaatagtaaatatcatgtcaaaattttcatcttcgccattattctttaggagaatttagaatgttccgaataaaatatggatagaaaagaagttcccaaagtcatccaattttctacaaaatcttcaactttaagattgagagtgggtgttgtaaagttcttggaagatgagttgaaccccatggaggcgttggctaaagtactgaaagcaatctaaagacatggaggtggaaggcctgacgtagatagagaagatgctcttagctttcattgtctttcttttgtagtcatctgatttacctctctcgtttatacaactggttttttaaacactccttaacttttcaaattgtctctttctttaccctagactagataattttaatggtgattttgctaatgtggcgccatgttagatagaggtaaaatgaactagttaaaagctcagagtgataaatcaggctctcaaaaattcataaactgttttttaaatatccaaatatttttacatggaaaataataaaatttagtttagtattaaaaaattcagttgaatatagttttgtcttcaaaaattatgaaactgatcttaattatttttccttaaaaccgtgctctatctttgatgtctagtttgagacgattatataattttttttgtgcttaactacgacgagctgaagtacgtagaaatactagtggagtcgtgccgcgtgtgcctgtagccactcgtacgctacagcccaagcgctagagcccaagaggccggaggtggaaggcgtcgcggcactatagccactcgccgcaagagcccaagaggccggagctggaaggatgagggtctgggtgttcacgaattgcctggaggcaggaggctcgtcgtccggagccacaggcgtggagacgtccgggataaggtgagcagccgctgcgataggggcgcgtgtgaaccccgtcgcgccccacggatggtataagaataaaggcattccgcgtgcaggattcacccgttcgcctctcaccttttcgctgtactcactcgccacacacaccccctctccagctccgttggagctccggacagcagcaggcgcggggcggtcacgtagtaagcagctctcggctccctctccccttgctccat

SEQ ID NO: 36 - DNA, 1500 bp, Zea mays
cgataagaacaatgttggacacaacttaagtctgttttacaacaatgtctctcaaaactatagttttacaatattatactttgcaattatcatgacaataatgtagtttcggtagctccaaaaatacagtagttttgagaaacattgtttagatacaatattataaatcatgtattagacaaaagatagccatgccattaaaactttgaattggactgtagttttttcaatactccaaaaatattatggtacctagaatacgatgtctagaaaacatattttttaaaatgcaaccaaacatcatatgacataaataatatagtatttttttgaaaaccatggtattacctaaaaactacagaatacttcattctgaaataggtcctaacaagttgcagcagctaggtcgtacatcagcaaatagctacttcatcaatctcagaataaacatattttatagatgagttaaactaaaaatatagaagaacaacgtacacgcgttgaatcacaacgtagcgcgatatccattcaactttttggaagtttttactgagcacaaattcgaaaatgggaagcgccacgtaacacgagcgctgggccaatttctgccagtgccagttatcccggcccacatccaatcctggggaagacgcgaacccggctccgcggcacgagttgtccgcacgtacggcacgtcggggctggctcgtccgcccgcgagtgggaggccactgtttcctctgcctcaccgggtcgtgtggcggaggggcgtggggccatggttcgcagcgcggggcgacgagcgcgctcctcctctcgcgcagcgccagcgccaccccgcaccgtggctttatatacacccctcctcccaaccctaccgaatcatcactaccaccgctctctcttcctctcctccatctctcaacgcctgaagctcaccgcacctcccctcctcgccgcggatcccccactactccggtaaccgtctctccattcaccctgcctgctgtctcgctagaatcgcctgcctctgccagcgccgtgacgcgggggcgcggtatggctctcccagatccgcctggcattgctcgctcgggtcgtgccaggccgatctgatctcgcatttgctgcgcgctcctcctgctgcggatcccaccggatctcgctggaatcggagcgcgcgtctctttgaaatgccgcagatctgcgtgcttgcgcgcgtgatctaagtccgggcctttcgttaacgaaatggtccgatctgtggtttggtggaggcaatgccatggtttttccccgtgaattttttttgctgattttaggagcttttttctactgtcctatgttagtaggacaaaaaaaaagaaacatagattagcttcaataggcgccttttagaacagattctgtacagcaactcgtggaaacaaatctgcttccttaatgatgttgcttgttttaacaaatgcggcatcgggcgagcttttctgtaggtagaaa

SEQ ID NO: 37 - DNA, 1694 bp, Artificial Sequence
  Synthethic construct hybrid cab5/hsp70 intron promoter from maize
cacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtatggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcatacggtatttatttaagcacctgttgctgctatagggcacttgtattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgacgataagtcaagatcagatgcacttgttttaaatattgttgtctgaagaaataagtactgacagttttttgatgcattgatctgcttgtttgttgtaacaaaattttaaaataaagagttccctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcgg

SEQ ID NO: 38 - DNA, 1500 bp, Zea mays
tttaaatttggaacgtcgatccaacatctaacagaagcaccaattttacaaagaacccctttcaccttcctcacttggtgggacggttcttaatcaaattaactgcagccgctggtatacatgtacatgtgggcccgcctagcccggcacggcacaggcccacaaaaacacggtccacaaaagcacgacccacaaaagcacatatctaattatgggccgtgccgtgccagcacgtgtgcccagtcatcggcccacaattagttatgtgtgccaggccgacccaaatagcccaaaataccttaatatgccagaccggctcatatacatacaacagtaatacatcaacaaaacgtataaaatatatatatgaccaaaataaaactaagatgttttgtggatgcacattataaacctttggtcagaaagaaaaaaatattacaactagctcacaaaaaatatccagttctctgtttagtgtttaattgagtactatacatccatacagaataaatatacaatgatcatcatcactattcactatccatatctaggtattggttctcgatggcttattaaagctctagattctccaagttatgctagtcatgtgggctttgacagaccttagttaaatactgagtctatattttgtgggccttagttaaatgggtcgtggcaggccggcccgtgggcttgacttgaggcccaggcacggcccacaatgtgggccgtgccggcccatgcccacaattaggttgggcagtgccagatatgggccgtgccagaaattgtgtgctttgggccggcctattaggcacaacataaatgtacacctatagccgcatagccgctggatgtgagatgaatgtctcagatttaaaatgtgcacttgagcaccgtacctctttgaacaacagatatgttcctttaagattgatggtggaaaaaaattagtcagtacctcactgtatggcggcattgtttgattatttcagttcgcacccgttggaccttgctcattaaaaaagtttataccatggagtctttgcatgtagttgtgtagtaggggaagagtggcataggaggaatcacaacttcagctagcttctctagccttagggtatttttgtctttttgcagttcggtcttttcgcagccctgcgctgccccccctgtccgcctgtccctagacctgttttgcgtcggcggggaagacagttgacaggaaggacacgatcttcgtgtccgatgccgatcttcatgcgagcagcgagccactacgttgcgctgccagtgtcggctatggtatccaggcattcgttgtgcacgttgacgatgagctcgaagccggtccgggtgaacgcgagcagcacggtgaggtcaacgtcgtacatccgcacgtcgatgctgaggccagccagcagcggcatgacagattgcggcgtcaggagattgtgccagtaggtggcggggctgggggcagaccggcaggcgaggcct

SEQ ID NO: 39 - DNA, 1500 bp, Zea mays
caaaattttctattttttaaaaaatatgaattctagatttgggattgaacacatctaggctacaacgttgaattgatgaacaatagtgcttgttaataaattgctcacattcacattgtcgctcttacttcaaccatcatacatccatctacagtggtcacccatatttaatcctatggactaaagatgacagatgaacttctctcgttatatatatcactgtcctacatatatgagaaatgatatgtcctaaactcacctaaaaacaacaacatagtttaaatttaatcatagatgagcctacagaggtcgaacgtgatttggaaacatagctctattgttctctatctcatgcataaatatggtgcaatgaagaatattagggttatgatgtcgaaatctcactcgaactcgtgcctcatcataaatagcacactatcaattgttctatggctgttcaaatagggacaatcttgaaacaacatttctcacatgtaaaacgttgtgaagtatgccaactgaaacggatgacacatacacttcgtgaaccaatcgatattttacttgcttctatgttaaataatgttataatacaatattttattcaaatgctaaaacttattactagataaaaataaaatttaattatcttcaaaaactaaccaatagatattccatcataactacatttaccaaactaatatactaaaaaatataggataattactaaattaatcgtgcaataatcagtatttatgagattgataattttaaattttgtgggctacaaacaaaaattaaaacttacttttcaagttggagataagaacaatggtagacgtagctcgggatggtatggcgtcggtgcagacggttaccctttgtgcgaagtggcgcgggcacgagggtggggacttggtacatgcatgagagagaggaagaacgaaacaacttctcaaattaaagcatatgaaaatcacctaatttttgtctgtcggtggaaactaataactagtttttattatcttttttaataaggatccacgaaaattatttttgaccgatgaaaatcctggatcttcgtattatgtttcgccttttcccgactctttgcatgctagatttccatgcttggactaaaacgaagataataaaaccaatctatcattttcacacgatgtattcatacttgcaatagataaaccactactccgacgggatttgctttctgacctctgaaatcttggaaggattatgtgtctacacttctcgatcgaggggaaaaagtcgtagtaccaagttgtagttaaatttgtttcttcgatgacaaaacaaaggagaggggcccgcgcggcgcagcgcagcgcagttggctggttccggaacacgaaaaccaagcacactccaccagctgccatccaccgggttggatggagattacaatactcgaatagtcagccagccagccggcttgaacgtgcagttttcccctataaaacg

SEQ ID NO: 40 - DNA, 1500 bp, Zea mays
acacttgctctcttcgcgtggtcatttagcccccgaacattccaagaaaaaatagcacatttttgattcataaggtaaagactgccactccacttaacacagcacgctgccaccacacatggattagcaggagagcctgctgtaaaatcctaacaggagggagaacctccaaacaagggttcgccgagcaaaaacacagcccgaccacaaccgacaacctgaaagaacaacagagatacacaggcatgctgggggacctagaccagcgcccagaagtaataacgccagcggagatacaaccgctccgagagagcctgaccatctgagaacacattggtcaccaaaagcaccaccaaccggcctagacaaagcagctcagttgacccccgcctcgacatcttcgatggccggcatcacctttctccccttctttttattcttcgctgtcttcaccttgtcttgatttaacagctccatgattgcatccatttgcttcttggagagaggctttgtgagaaggcttgtcatctgctcaaatgactcatcaaagttagtacattttgaagaactaattattattatatagaatgcactgcacatatattactattaccagttttcttgggcacagcagaaaacatgcacacgcagatagaaaaaggagaggccataaaccaaaaggctttaagaatatatgtaaagatatgtctaaatatatggctatatctggttaagcaagataacagggctctggtcatcagtagtagtggccttttgcccttgcccctctctctcacctctcttttctcagccttgcttccgatggatcccatcccactgccatcctttctttcccttgcgcgcattgcctagccggccggccggcctgctattaaaccactttacccgccccctctcgctcacgctcgacgcagctcccttttccttgtttgcttattgcaagtctctgcaagaacctgctagagaggaacaaggtagagtagtatcgcttttttccatctaggttatctctttttacatgaaaaatttcagccgtatttcgttctccatcagtcctgcgataatatatacgcgcgtcttgtgtgatccggcatatgtatagttcctgctaactgatcgagatcgctctcgtttgtactttctccctttgaggaaagagtttccccttttctgtgcttcaagttcttgtaaggaaaaccatgcctgccagcttcttctgctacttgtatgatgattcttatttgcttattacttgatttccgttttttttcttgctttctatatgtatgtatctgggctgtcttcccctgcgtctcgttactgctaagctttggaaggtttcaactctttgtatacgatgaggtttctgctcctagtagcagatccgcgcatatgactagatgtttgaggaaaagaaaagggcaagacgctatatatatatgcagcacgcagtcgcacatatattcagttttccaatct

SEQ ID NO: 41 - DNA, 1500 bp, Zea mays
gattcgcgcgtgctggaactcgggattggattcatgcgtgctggaacttggaagtctggagtggactttggaagcctggattccagaacaagaactcaagaagtctaggagccgccgagcaggtagggaattagggaaataaagagaagaggcggctggcgttcgacgttccatcttcagtagaggcggctggcgtttcaactccctgtagtcgggccgcctgccaaaaaagcccacgaaggcaggaaatcaaaaaatctaggtcctaaacctagtcgcgcagaaccggctaatcgagcgactaatcgaccctaatcgtcgactagtcggacggccagggcgattaggtactctaatcgagtcggttgtgctaatcgagctctgctaaccgactagcccgaccgcgattagtcagatgacttgaaaacaaagatagagacatacttttttatattctttgcattgttttgtttctatccaaaactgctatttagaaattggaaaatctgcacattgaaaaatctaatggattagatatgttgatttgtttttattcacgagcataatcaaataaattagatttagaattggactgcacgcagtgaactactgaactgaactgtgttcaataatttaaatactcacggctgagccgtgagctgtaggctggagcacaagcacgagccagcaccgagcggcggagcactggagcagcaggcgagcagggaggcggccaggcgggagcagccagccagcaagcaggcagcagcggagcagcccacagccgagcgcccaagctggagctgctgcagagcctgcagcgtgccgctgcgcgccggcaggacaggagcggccgagcgggagtgcaggactgtggcctgcgggacgcggggatgggcggacggcgtagcgcttacagtccgcggacagcggactcacggtggcggctaagatagtgagaccgatgacctaatctctatttggaccggttcaaggttttgcccagttaatattggaccatattgggccttccgcccctgctcgcaagacacactgaacaaagaatccacacggctctccaaaagatagagagataattcacatgcttctctctctctgaaaaaaaggaacttgcatggttgacacggaaaacgtcattaaacgcgcacgtggctgcaaatgcaacgtaacagatccatcatctatccatccatagaatcagacggccacagaaggcaacgaccgtgtgcctgtccaccggcgcaggtggcccacagacgcccgtgcgattcatccgtctcggcccaccaaccacgggaggggccccagggccctccttagtccttacaaataccggcagcagcatcacccggccaccaccacccacccgttttatccacgcacggcgtcgaacaccccgcggtcgctcacgtgaggcgccaccccgcgcacccagtcagcgcccgcctccaccacccacccacacgacaaaaatccgcc

SEQ ID NO: 42 - DNA, 231 bp, Arabidopsis thaliana
atggcaatggctgttttccgtcgcgaagggaggcgtctcctcccttcaatcgccgctcgcccaatcgctgctatccgatctcctctctcttctgaccaggaggaaggacttcttggagttcgatctatctcaactcaagtggtgcgtaaccgcatgaagagtgttaagaacatccaaaagatcacaaaggcaatgaagatggttgctgcttccaagcttagagcagttcaa

SEQ ID NO: 43 - DNA, 1731 bp, Artificial Sequence
  Synthetic construct mitochondrial targeting seq fused to codon optimized Corynebacterium mqo seq
atggcaatggctgttttccgtcgcgaagggaggcgtctcctcccttcaatcgccgctcgcccaatcgctgctatccgatctcctctctcttctgaccaggaggaaggacttcttggagttcgatctatctcaactcaagtggtgcgtaaccgcatgaagagtgttaagaacatccaaaagatcacaaaggcaatgaagatggttgctgcttccaagcttagagcagttcaatctgattcacctaagaacgcacctagaatcaccgatgaggctgatgtggttcttatcggagctggtattatgtcaagtaccctcggagctatgcttagacaattggagccttcttggactcagattgttttcgagagacttgatggtcctgctcaagaatcttcttctccatggaataacgctggaactggtcattctgctctttgtgaattgaattatacaccagaggttaagggtaaagttgaaattgctaaggctgttggaatcaacgagaaatttcaagtttctagacagttctggtcacatcttgttgaagagggagttttgtctgatcctaaggaattcattaatcctgttccacatgtttctttcggacaaggtgctgatcaggttgcttatatcaaggctagatacgaggctcttaaagatcatccattgttccagggtatgacttacgctgatgatgaagctacttttacagagaagcttcctttgatggctaaaggaagagatttttctgatccagttgctatttcttggatcgatgaaggtactgatattaactatggagctcaaacaaaacagtaccttgatgctgctgaagttgagggtacagagatcagatacggacatgaggttaagtctattaaagctgatggtgctaagtggatcgttactgttaaaaacgttcatacaggagatactaagacaattaaagctaacttcgttttcgttggtgctggaggttacgctttggatcttcttagatcagctggaatccctcaagttaagggatttgctggtttcccagtttctggtctttggttgagatgtacaaatgaggagcttattgagcagcatgctgctaaggtttatggaaaagcttctgttggtgctcctccaatgtctgttcctcatttggatactagagttatcgaaggagagaaaggtcttttgtttggtccttatggaggttggacaccaaagttccttaaggaaggatcttaccttgatttgtttaagtctatcagacctgataacatcccatcttatttgggagttgctgctcaagagttcgatcttactaagtacttggttacagaagttcttaaggatcaagataagaggatggatgctttgagagaatatatgccagaggctcagaacggagattgggaaactattgttgctggacaaagagttcaggttatcaagcctgctggatttccaaaattcggttctcttgagttcggaactacattgattaataactctgaaggtacaatcgctggacttttgggtgcttctcctggagcttctattgctccatctgctatgatcgaacttttggagagatgttttggagatagaatgattgagtggggagataagcttaaagatatgatcccttcttacggaaagaagttggcttctgaaccagcattatttgaacaacagtgggcaagaacacagaaaacattgaagttagaggaggcatga

SEQ ID NO: 44 - protein, 576 aa, Artificial Sequence
  Synthetic construct mitochondrial targeting seq fused to codon optimized Corynebacterium mqo
MAMAVFRREGRRLLPSIAARPIAAIRSPLSSDQEEGLLGVRSISTQVVRNRMKSVKNIQKITKAMKMVAASKLRAVQSDSPKNAPRITDEADVVLIGAGIMSSTLGAMLRQLEPSWTQIVFERLDGPAQESSSPWNNAGTGHSALCELNYTPEVKGKVEIAKAVGINEKFQVSRQFWSHLVEEGVLSDPKEFINPVPHVSFGQGADQVAYIKARYEALKDHPLFQGMTYADDEATFTEKLPLMAKGRDFSDPVAISWIDEGTDINYGAQTKQYLDAAEVEGTEIRYGHEVKSIKADGAKWIVTVKNVHTGDTKTIKANFVFVGAGGYALDLLRSAGIPQVKGFAGFPVSGLWLRCTNEELIEQHAAKVYGKASVGAPPMSVPHLDTRVIEGEKGLLFGPYGGWTPKFLKEGSYLDLFKSIRPDNIPSYLGVAAQEFDLTKYLVTEVLKDQDKRMDALREYMPEAQNGDWETIVAGQRVQVIKPAGFPKFGSLEFGTTLINNSEGTIAGLLGASPGASIAPSAMIELLERCFGDRMIEWGDKLKDMIPSYGKKLASEPALFEQQWARTQKTLKLEEA

SEQ ID NO: 45 - DNA, 169 bp, Zea mays
accaaccattttcgttagcaaagaaattaagattttttttttcctttcaatcttggctttcagggttgttggttgttgtgtagtgaaaataattgtgtccctatgttgaatgctcaaatgaataaatcaagatcccatatattatattagcagagcagaaggcagtgtt

SEQ ID NO: 46 - DNA, 552 bp, Artificial Sequence
  Synthetic construct bar gene
atgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttga

SEQ ID NO: 47 - DNA, 623 bp, Zea mays
gtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgat

SEQ ID NO: 48 - DNA, 523 bp, Glycine max
gcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaaggcgcgccagt

SEQ ID NO: 49 - DNA, 1216 bp, Artificial Sequence
  Synthetic construct hygromycin gene including cat-1 intron from bean catalase-1 gene
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 50 - DNA, 319 bp, Glycine max
gttataatcatggagtgtgaaagctggaccagggaaattactatttatacaaatactacaaaaataccatctagtggttgaggaactttcatttcctactctttaccatccttttatctatcttgtttttgtgttttcctttctttggtatgttgagataagagcatgaaggctagcaagatatgtaagattcttttttttttctcccgttctgttgtagaagagatgtgaattgttacctatttggtttggttgaattaacaaattcttttcatgaagtattctgattaacatagtggtacggtacgtgcttgattta

SEQ ID NO: 51 - DNA, 13241 bp, Artificial Sequence
  Synthetic construct plasmid pMBXS1276
tgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggcctcagcttaattaatacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcagatctaaaatggcaatggctgttttccgtcgcgaagggaggcgtctcctcccttcaatcgccgctcgcccaatcgctgctatccgatctcctctctcttctgaccaggaggaaggacttcttggagttcgatctatctcaactcaagtggtgcgtaaccgcatgaagagtgttaagaacatccaaaagatcacaaaggcaatgaagatggttgctgcttccaagcttagagcagttcaatctgattcacctaagaacgcacctagaatcaccgatgaggctgatgtggttcttatcggagctggtattatgtcaagtaccctcggagctatgcttagacaattggagccttcttggactcagattgttttcgagagacttgatggtcctgctcaagaatcttcttctccatggaataacgctggaactggtcattctgctctttgtgaattgaattatacaccagaggttaagggtaaagttgaaattgctaaggctgttggaatcaacgagaaatttcaagtttctagacagttctggtcacatcttgttgaagagggagttttgtctgatcctaaggaattcattaatcctgttccacatgtttctttcggacaaggtgctgatcaggttgcttatatcaaggctagatacgaggctcttaaagatcatccattgttccagggtatgacttacgctgatgatgaagctacttttacagagaagcttcctttgatggctaaaggaagagatttttctgatccagttgctatttcttggatcgatgaaggtactgatattaactatggagctcaaacaaaacagtaccttgatgctgctgaagttgagggtacagagatcagatacggacatgaggttaagtctattaaagctgatggtgctaagtggatcgttactgttaaaaacgttcatacaggagatactaagacaattaaagctaacttcgttttcgttggtgctggaggttacgctttggatcttcttagatcagctggaatccctcaagttaagggatttgctggtttcccagtttctggtctttggttgagatgtacaaatgaggagcttattgagcagcatgctgctaaggtttatggaaaagcttctgttggtgctcctccaatgtctgttcctcatttggatactagagttatcgaaggagagaaaggtcttttgtttggtccttatggaggttggacaccaaagttccttaaggaaggatcttaccttgatttgtttaagtctatcagacctgataacatcccatcttatttgggagttgctgctcaagagttcgatcttactaagtacttggttacagaagttcttaaggatcaagataagaggatggatgctttgagagaatatatgccagaggctcagaacggagattgggaaactattgttgctggacaaagagttcaggttatcaagcctgctggatttccaaaattcggttctcttgagttcggaactacattgattaataactctgaaggtacaatcgctggacttttgggtgcttctcctggagcttctattgctccatctgctatgatcgaacttttggagagatgttttggagatagaatgattgagtggggagataagcttaaagatatgatcccttcttacggaaagaagttggcttctgaaccagcattatttgaacaacagtgggcaagaacacagaaaacattgaagttagaggaggcatgaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccac

SEQ ID NO: 52 - DNA, 6168 bp, Artificial Sequence
  Synthetic construct T-DNA insert from corn transformation
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacctagctaataacttcgtatagcatacattatacgaagttatagtcctactagttagttaggcgatcgccctatttacaaaaataagggctcatgtatatctatttagatattgacatttgtcaaacaacagataagctatatatgatatggcacttagtcaattttgacatttagttatcaattaggctggttagcttaatcattcggactaggcaaaaattttgggctaaggatattcaatatggctaaaatatttaaaaagaacaacttcaattcactcctaatccaaacgagggaaaaaccttttcatggactttgttactattccttttttatttatgaagaaattaattgtatgactgtgaaataatgactgcagttgttgtgctcactgaaagagcaaggagcgcaaggagggcaaatatcatgggctccaaatatttccttacatgttgtattatagggtcatgcacaatttgggttctagcaaaaaacatatgcaccaaatttttactaaaaacaataactaagaggtaagacttcgcgcgaacaactttgcacacacatataggtttactcatgaacttatattatgttttgcttggtatatgtgcacctgatttgagtgttcacggtcaaagcatatgtgccaatcactttctcaaaagaacatatcacagatttaagtactcaactacaagcacgtacagtcacatctatcttataaacacacaagcacacatccaaacaagaacattcaagcggttcgagctggcacatctttggcttggtgaagtatgcggtattgtctacatattaaccaagcatgtatctttaattttgatttgatgttttcaaatgaatatacaaagaacactcttaagaaattgactcaaattaattctcaaacaatatattgattatttattggaagttgcacacgccgatatatatgagacctttgttatgaagtacaactcatatgtattagatcttggatttaaatgcgatcgggccagaatggcccggaccgaagcttgcatgcctgcagattcgcgcgtgctggaactcgggattggattcatgcgtgctggaacttggaagtctggagtggactttggaagcctggattccagaacaagaactcaagaagtctaggagccgccgagcaggtagggaattagggaaataaagagaagaggcggctggcgttcgacgttccatcttcagtagaggcggctggcgtttcaactccctgtagtcgggccgcctgccaaaaaagcccacgaaggcaggaaatcaaaaaatctaggtcctaaacctagtcgcgcagaaccggctaatcgagcgactaatcgaccctaatcgtcgactagtcggacggccagggcgattaggtactctaatcgagtcggttgtgctaatcgagctctgctaaccgactagcccgaccgcgattagtcagatgacttgaaaacaaagatagagacatacttttttatattctttgcattgttttgtttctatccaaaactgctatttagaaattggaaaatctgcacattgaaaaatctaatggattagatatgttgatttgtttttattcacgagcataatcaaataaattagatttagaattggactgcacgcagtgaactactgaactgaactgtgttcaataatttaaatactcacggctgagccgtgagctgtaggctggagcacaagcacgagccagcaccgagcggcggagcactggagcagcaggcgagcagggaggcggccaggcgggagcagccagccagcaagcaggcagcagcggagcagcccacagccgagcgcccaagctggagctgctgcagagcctgcagcgtgccgctgcgcgccggcaggacaggagcggccgagcgggagtgcaggactgtggcctgcgggacgcggggatgggcggacggcgtagcgcttacagtccgcggacagcggactcacggtggcggctaagatagtgagaccgatgacctaatctctatttggaccggttcaaggttttgcccagttaatattggaccatattgggccttccgcccctgctcgcaagacacactgaacaaagaatccacacggctctccaaaagatagagagataattcacatgcttctctctctctgaaaaaaaggaacttgcatggttgacacggaaaacgtcattaaacgcgcacgtggctgcaaatgcaacgtaacagatccatcatctatccatccatagaatcagacggccacagaaggcaacgaccgtgtgcctgtccaccggcgcaggtggcccacagacgcccgtgcgattcatccgtctcggcccaccaaccacgggaggggccccagggccctccttagtccttacaaataccggcagcagcatcacccggccaccaccacccacccgttttatccacgcacggcgtcgaacaccccgcggtcgctcacgtgaggcgccaccccgcgcacccagtcagcgcccgcctccaccacccacccacacgacaaaaatccgccatggcgatggccgtgttcagaagggagggtaggaggctcctcccctcaatagccgccagacccattgcggcgataaggagccccctctcgagcgaccaagaggagggactcctcggtgtcaggtctatcagcacccaggtcgtccgcaaccgcatgaaaagcgtcaagaacatccagaagatcaccaaggccatgaagatggtcgccgcctcaaagctcagggccgtccagagcgacagccccaagaacgcaccacggatcaccgatgaagcagacgtggtcctcataggcgccgggatcatgtcatcaacactcggcgccatgctcaggcagctcgagccaagctggacgcagatcgtgttcgagaggctcgacggaccagcccaggagtcgagcagcccctggaacaacgcaggcactggacactcagcactctgcgagctcaactacacccccgaggtcaagggaaaggtcgagatcgccaaagccgtcggaatcaacgaaaagttccaagtctccaggcagttctggagccacctcgtcgaagaaggcgtgctcagcgacccaaaggagttcatcaaccccgtcccccacgtcagcttcggacagggagcagaccaggttgcatacatcaaagcccgctacgaagccctcaaggaccacccactcttccagggcatgacatacgccgacgacgaggccaccttcaccgagaagctcccactcatggcgaagggaagggacttctccgacccagtcgcaatctcgtggatagacgagggcacggacatcaactacggcgcccagacaaagcagtacctcgacgcagcagaggtcgaggggacagagatccgctacgggcacgaagtcaagagcatcaaagccgacggcgcaaagtggatagtcaccgtcaagaacgtccacacaggcgacacaaagaccatcaaggccaacttcgtgttcgtcggagcaggcggttacgcacttgacctcctcaggtcagcaggaatcccccaggtgaagggattcgcaggcttcccagtcagcggactgtggttgaggtgcaccaacgaggagctcatagagcagcacgccgccaaggtctacggcaaggcttctgtcggtgcacctccaatgagcgtgcctcacctcgacacacgcgtcatcgaaggagagaaggggctcctcttcggcccatacggtggatggacccccaagttcctcaaggagggcagctacctcgacctcttcaaatccatccgccccgacaacatcccaagctacctgggagtggcagcccaagagttcgacctcacaaagtacctcgtgaccgaggtcctcaaggaccaggacaagcgcatggacgcactccgcgagtacatgccagaggcccaaaatggcgactgggaaaccatcgtcgcgggacaaagggtccaagtcataaaacccgcggggttccccaagttcggcagcctcgaattcggcaccaccctcatcaacaacagcgagggaactatcgcagggctcctcggagcctcaccaggcgcatccatagccccttcggccatgatcgagctcctcgaacgctgtttcggggacaggatgatcgagtggggcgacaagctcaaggacatgatccccagctacggcaagaaactcgcctccgagcctgcactcttcgagcagcagtgggcaaggacccaaaagacactcaaactcgaggaggcctaattcgaacgcgtaggtacctacgtactagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaggcctcccggggcacttaggagcgaagactaacggttagctagggctgatacctaaaccttagtcactagcagcgtctactagcggcgcgccacttacgcttgattccgatgacttcgtaggttcctagctcaagccgctcgtgtccaagcgtcacttacgattagctaatgattacggcatctaggaccgactagctagctagctaagagctcggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca